SEQUENCE LISTING (26 sequences)

SEQ ID NO: 1 - DNA, 57 bp, synthetic construct
  source 1..57: mol_type = other DNA
aagcagtggtatcaacgcagagtacttttttttttttttttttttttttttttttvn

SEQ ID NO: 2 - DNA, 53 bp, synthetic construct
  source 1..53: mol_type = other DNA
  misc_feature 1: note = A at position 1 is biotinylated
acgagcatcagcagcatacgattttttttttttttttttttttttttttttvn

SEQ ID NO: 3 - DNA, 30 bp, synthetic construct
  source 1..30: mol_type = other DNA
  modified_base 30: mod_base = OTHER; note = LNA-modified guanosine
  modified_base 29: mod_base = OTHER; note = riboguanosine
  modified_base 28: mod_base = OTHER; note = riboguanosine
aagcagtggtatcaacgcagagtacatggg

SEQ ID NO: 4 - DNA, 30 bp, synthetic construct
  source 1..30: mol_type = other DNA
  misc_feature 1: note = A at position 1 is biotinylated
  modified_base 29: mod_base = OTHER; note = Riboguanosine
  modified_base 28: mod_base = OTHER; note = Riboguanosine
  modified_base 30: mod_base = OTHER; mod_base = OTHER; note = riboguanosine
agagacagattgcgcaatgnnnnnnnnggg

SEQ ID NO: 5 - DNA, 23 bp, synthetic construct
  source 1..23: mol_type = other DNA
aagcagtggtatcaacgcagagt

SEQ ID NO: 6 - DNA, 44 bp, synthetic construct
  source 1..44: mol_type = other DNA
  modified_base 42..44: mod_base = OTHER; note = phosphorothioate bonds linking residue 42 with 43 and residue 43 with 44
tcgtcggcagcgtcagatgtgtataagagacagattgcgcaatg

SEQ ID NO: 7 - DNA, 21 bp, synthetic construct
  source 1..21: mol_type = other DNA
  modified_base 19..21: mod_base = OTHER; note = phosphorothioate bonds linking residue 19 with 20 and residue 20 with 21
acgagcatcagcagcatacga

SEQ ID NO: 8 - DNA, 26 bp, synthetic construct
  source 1..26: mol_type = other DNA
tcgccgtatcattcaagcagaagacg

SEQ ID NO: 9 - DNA, 33 bp, synthetic construct
  source 1..33: mol_type = other DNA
tcgtcggcagcgtcagatgtgtataagagacag

SEQ ID NO: 10 - DNA, 33 bp, synthetic construct; identical to SEQ ID NO: 9
  source 1..33: mol_type = other DNA

SEQ ID NO: 11 - DNA, 34 bp, synthetic construct
  source 1..34: mol_type = other DNA
gtctcgtgggctcggagatgtgtataagagacag

SEQ ID NO: 12 - DNA, 34 bp, synthetic construct
  source 1..34: mol_type = other DNA
ccgtatcattcaagcagaagacggcatacgagat

SEQ ID NO: 13 - DNA, 33 bp, synthetic construct
  source 1..33: mol_type = other DNA
ctgtctcttatacacatctgacgctgccgacga

SEQ ID NO: 14 - DNA, 18 bp, synthetic construct
  source 1..18: mol_type = other DNA
aactagagaacccactgc

SEQ ID NO: 15 - DNA, 18 bp, synthetic construct
  source 1..18: mol_type = other DNA
aacttcagggtcagcttg

SEQ ID NO: 16 - DNA, 20 bp, synthetic construct
  source 1..20: mol_type = other DNA
agcactctgattgtgccttc

SEQ ID NO: 17 - DNA, 28 bp, synthetic construct
  source 1..28: mol_type = other DNA
ggacacaatttgacctattaacctattc

SEQ ID NO: 18 - DNA, 18 bp, synthetic construct
  source 1..18: mol_type = other DNA
atgctgagcaacaccaca

SEQ ID NO: 19 - DNA, 20 bp, synthetic construct
  source 1..20: mol_type = other DNA
aaactcaccttcctccatcc

SEQ ID NO: 20 - DNA, 21 bp, synthetic construct
  source 1..21: mol_type = other DNA
ggtttccacagctgtagttga

SEQ ID NO: 21 - DNA, 22 bp, synthetic construct
  source 1..22: mol_type = other DNA
gacgctcaatatcgaggtttct

SEQ ID NO: 22 - DNA, 22 bp, synthetic construct
  source 1..22: mol_type = other DNA
cgatattgagcgtccaacctat

SEQ ID NO: 23 - DNA, 22 bp, synthetic construct
  source 1..22: mol_type = other DNA
gtctggaattctgtcaggtcaa

SEQ ID NO: 24 - DNA, 19 bp, synthetic construct
  source 1..19: mol_type = other DNA
ttgccaccatcaagaccaa

SEQ ID NO: 25 - DNA, 21 bp, synthetic construct
  source 1..21: mol_type = other DNA
cacacagctctctgtaccttg

SEQ ID NO: 26 - RNA, 14 nt, synthetic construct
  source 1..14: mol_type = other RNA
ggcacttcggtgcc

(19) United States
(12) Patent Application Publication
(10) Pub. No.: US 2026/0139245 A1
(43) Pub. Date: May 21, 2026

(54) METHODS AND USES OF RIBONUCLEASE INHIBITORS

(71) Applicant: Sequrna AB, Järfälla (SE)

(72) Inventors: Björn L E Reinius, Järfälla (SE); Antonio Lentini, Vikingstad (SE); Carol J. Noble, Stockholm (SE)

(21) Appl. No.: 18/862,118

(22) PCT Filed: May 4, 2023
(86) PCT No.: PCT/EP2023/061876

§ 371 Date: Oct. 31, 2024

(30) Foreign Application Priority Data
May 5, 2022 (GB) 2206554.4

(51) Int. Cl. C12N 15/10

(52) U.S. Cl. CPC C12N 15/1065

(57) ABSTRACT

There are provided agents for the inhibition of RNases as well as methods of their use. The agents are especially suited for RNA sequencing, hereunder single-cell RNA sequencing.

### FIELD

The invention relates to a class of chemical agents that, within specific concentration ranges, can be used as ribonuclease (RNase) inhibitors in the preparation of single-cell RNA-sequencing libraries, bulk RNA sequencing libraries and in situ RNA-sequencing, without the need of separating the RNAse inhibitory agent from the sample during preparation and without negatively affecting the quality of the sequencing library; and kits and products comprising the same. Furthermore, the use of these agents in single-cell RNA-sequencing lysis buffers, together with their property of being thermostable, enable new experimental workflows and extend the workable time frame of RNA-sequencing library preparation.

### BACKGROUND

Biomedical research and biotechnology rely on polymeric nucleic acids. Yet during their storage and use, nucleic acids encounter nucleases that degrade nucleic acid. For example, human skin is an abundant source of nucleases that can be transferred accidentally to surfaces and solutions, and biological samples analysed for nucleic acid content are themselves generally a source of them. A ribonuclease (commonly abbreviated RNase), is a type of nuclease that catalyzes the degradation of RNA into smaller components.

Ribonuclease inhibitor (RI) is a large 50 kDa protein present in the cytosol of mammalian cells. RI forms extremely tight complexes with certain RNases and controls the activity of RNases. Inhibitors of ribonuclease, both chemical and biological, are useful in a variety of molecular biology applications where RNase contamination is a potential problem. Examples of these applications include mRNA isolation and purification, storage, reverse transcription of mRNA, RNA Sequencing (RNA-seq), and in situ RNA-sequencing. One solution is the pre-treatment of samples and solutions with diethylpyrocarbonate (DEPC), which is effective for ribonuclease inhibition. However, DEPC and other similar chemicals are known carcinogens and require caution and training for their use. These chemicals also react quite readily with amine, thiol, and alcohol groups so some solutions (e.g., primary amine containing compounds such as Tris) cannot be treated with DEPC at all. Finally, DEPC must be inactivated by autoclaving post-treatment, but DEPC residues may still interfere with downstream enzymatic reactions such as reverse transcription (RT) and polymerase chain reaction (PCR).

The capture of intact RNA is a requisite of RNA-sequencing (RNA-seq) methods to accurately record the transcriptome of the analyzed sample material. Single-cell RNA-sequencing (scRNAseq) is particularly sensitive to RNA degradation and detection dropout due to the miniscule copy numbers of individual transcripts present in a single cell. Due to the low RNA copy numbers in a single cell, RNA purification is practically unworkable in scRNAseq library preparation, and any RNA-protective agent added to the single-cell sample must be directly compatible with downstream reaction steps.

Thus, the use of in vitro synthesized biological RNase inhibitors (i.e. recombinant RI proteins) is a nearly universal feature during cell-lysis and storage as well as reverse transcription in single-cell RNA-seq protocols. However, the use of recombinant inhibitors is inconvenient due to its relatively high cost fraction to the library, but also due to its degradability; which may introduce batch variation in library yield and quality due to production lot, storage time, and temperature conditions for the inhibitor. Thus, there is a need for ribonuclease inhibitors for applications which require capture of intact RNA. If thermostable RNase inhibitors could be identified, this would enable new and simplified workflows, and may increase reproducibility and throughput of RNA-seq applications. Importantly, to satisfactorily replace recombinant RNase inhibitors in scRNAseq library preparation, such ribonuclease inhibitors must not only be capable of preserving cellular RNA in the lysis buffer but must be fully compatible with each of the following library preparation steps that are universal for all scRNA-seq, including reverse transcription and amplification by PCR, without introducing base errors or reducing sensitivity to detect RNA species in contained in the analysed sample material.

### SUMMARY

In its broadest aspect, the present invention relates to a method of using chemical RNase inhibitor as well as such chemical RNase inhibitors. The inhibitors may be used in applications where inhibition of RNase activity is desired, such as in RNA sequencing and hereunder single cell RNA-sequencing.

### DETAILED DESCRIPTION

Protein-based RIs are considered specific for RNase whereas chemicals with RNase inhibitory do in general also affect or inhibit other enzymes which are critical in the molecular biology application, such as reverse transcriptase and DNA polymerase. The inventors have found that certain chemicals are suitable for use in applications where inhibition of RNase activity is desirable such that certain specific concentrations do not negatively affect other enzymes.

Although there are potentially many chemical substances or treatments that may in principle inhibit RNase activity, these are generally expected to also negatively affect scRNAseq library yield as well as the quality and error rate in the final sequencing library. Thus, from a chemical compound being a potent RNase inhibitor it does not follow that the agent is also suitable RNase inhibitor in scRNAseq.

The inventors have surprisingly found that recombinant biological RNase inhibitors widely used in scRNAseq library preparation can be replaced by a sulfonated polymer, a sulfonated monomer, or a carboxylated polymer, supplied in defined concentration ranges, yielding bulk RNA-seq and scRNAseq libraries of equal or superior quality at virtually no cost for RNA inhibition. Moreover, the thermostability of chemical RNase inhibition means it need not be supplemented twice during RNA collection and cDNA library preparation (i.e. in the cell lysis or collection step and in the reverse transcription step), which enables new and simplified workflows, increasing reproducibility and throughput of RNA-seq. For, example, stable premade sample collection buffers can be made, frozen, thawed, and kept at room-temperature for extended periods of time, or even subjected to high-temperature conditions before use.

These findings are particularly surprising since, for example, an exemplary agent of the invention, poly(vinylsulfonic acid) (PVSA), was known to be a strong inhibitor of catalysis by RNA polymerase and reverse transcriptase (Chambon et al., 1967; Althaus et al., 1992), which the inventors furthermore experimentally confirmed in the experiments presented in the accompany Examples. The inventors identified that surprisingly low titres of PVSA form a concentration range (optimally 0.1-120 μg/mL in lysis buffer) in which PVSA can effectively replace normally used recombinant RI in scRNAseq library preparation without negatively affecting the quality of the scRNAseq library. Importantly, this application as well as the workable concentration range is contrasting to a study utilizing PVSA in cell-free protein translation at >40-fold higher concentration with decoupling of in vitro transcription and translation by a purification step (Earl 2018 Bioengineering PMID: 28662363).

As it appears from the above disclosure, other chemicals may also be suitable for use as RNase inhibitors.

In a first aspect, the invention provides a method of preparing a cDNA sequencing library from RNA, characterised in that the method includes the use of an agent selected from the group consisting of: a sulfonated and/or carboxylated polymer; a sulfonated and/or carboxylated monomer; and a functionalised polysaccharide.

In an embodiment the agent is selected from the group consisting of: a sulfonated and/or carboxylated vinyl polymer; a sulfonated and/or carboxylated monomer; and a functionalised polysaccharide.

In an embodiment the agent is selected from the group consisting of: a sulfonated or carboxylated vinyl polymer; a sulfonated or carboxylated monomer; and a functionalised polysaccharide.

In an embodiment the agent is selected from the group consisting of: a sulfonated and/or carboxylated vinyl polymer; a sulfonated and/or carboxylated vinyl monomer; and a functionalised polysaccharide.

In an embodiment the agent is selected from the group consisting of: a sulfonated or carboxylated vinyl polymer; a sulfonated or carboxylated vinyl monomer; and a functionalised polysaccharide.

In an embodiment the agent is selected from the group consisting of: a sulfonated or carboxylated polymer; a sulfonated or carboxylated monomer; and a functionalised polysaccharide.

The inventors have surprisingly found that an exemplary polymer, PVSA, can efficiently replace recombinant RNase inhibitors in scRNAseq library preparation, preventing RNA degradation. Surprisingly, given PVSA's previously known inhibitory effect on both reverse transcriptase and DNA polymerase, the yielded scRNA-seq libraries have quality measures on par with scRNA-seq libraries generated using conventional recombinant RNase inhibitor under specific concentration ranges identified by the inventors.

By “polymer” we include the meaning of any of a class of natural or synthetic substances that are multiples of simpler chemical units called monomers. In an embodiment, the polymer is a non-protein polymer. In an embodiment, the polymer is a non-biological polymer. By “non-biological” we include the meaning of a molecule or agent not normally found in a biological system.

In a particular embodiment, such polymers (i.e. sulfonated and/or carboxylated polymers, as described herein) are vinyl polymers. In a further embodiment, the relevant monomers (i.e. sulfonated and/or carboxylated monomers, as described herein) are vinyl polymers.

By “vinyl polymer” we include the meaning of products from the polymerization of vinyl monomers. By “vinyl monomers” we include the meaning of monomers containing vinyl groups, i.e. small molecules containing carbon-carbon double bonds.

Salt forms of any of the polymers and polysaccharides described herein may also be used in the methods of the present invention. Any salt form used should not comprise a cation which inhibits any part of the method of the invention, such as the PCR reaction. Salts that may be used include acid addition salts and base addition salts. Examples of addition salts include those derived from mineral acids, such as hydrochloric, hydrobromic, phosphoric, metaphosphoric, nitric and sulphuric acids; from organic acids, such as tartaric, acetic, citric, malic, lactic, fumaric, benzoic, glycolic, gluconic, succinic, arylsulphonic acids; and from metals such as sodium, magnesium, potassium or calcium. In an embodiment, the salt form is a sodium salt.

It will be appreciated that for salt forms of the RNase-inhibiting polymer or monomer, the counter ion may be exchanged to another counter ion. Indeed, it is common knowledge in chemistry that a functional charged molecule can be paired with various counter ions.

As a specific example, sodium alginate (NaAlg) having sodium (Na⁺) as counter ion could be replaced by potassium alginate (KAlg) having potassium (K⁺) as counter ion.

Unsalted forms of the polymers and polysaccharides described herein may also be used in the methods of the present invention.

By “sulfonated polymer” we include the meaning of a repeated chain of molecules wherein a sulfonate residue appears at least once per unit in the chain. In an embodiment, the sulfonated polymer comprises one sulfonate group per unit.

By “carboxylated polymer” we include the meaning of a repeated chain of molecules wherein a carboxylate residue appears at least once per unit in the chain. In an embodiment, the carboxylated polymer comprises one carboxyl group per unit.

By “sulfonated monomer” we include the meaning of a compound that is non-repeating and that contains at least one sulfonate residue.

By “carboxylated monomer” we include the meaning of a compound that is non-repeating and that contains at least one carboxylated residue.

By “polysaccharide” we include the meaning of polymeric carbohydrates composed of repeating units, e.g. monosaccharides or disaccharides, linked together by glycosidic bonds. Polysaccharide compounds such as glycosaminoglycans are also included.

Polysaccharides are known in the art and include but are not limited to, cellulose, amylose, dextran, and heparin. Native heparin has a molecular weight ranging from 3 to 30 kDa, although the average molecular weight of most commercial heparin preparations is in the range of 12 to 15 kDa. Dextrans are available in multiple molecular weights ranging from 3 kDa to 2 MDa. The molecular weight of amylose varies between several thousand and one-half million daltons with a degree of polymerization of 1000-10,000 glucose units.

By “functionalised” we include the meaning that the polysaccharide comprises one or more acidic group. In an embodiment, the functionalised polysaccharide is a sulfated and/or carboxylated polysaccharide.

By “sulfated polysaccharide” we include the meaning of a chain of repeating units linked together by glycosidic bonds wherein a sulfate residue appears at least once per unit in the chain. The repeating unit may be a monosaccharide or a disaccharide.

In an embodiment, the sulfated polysaccharide comprises one sulfate group per monosaccharide.

By “carboxylated polysaccharide” we include the meaning of a chain of repeating units linked together by glycosidic bonds wherein a carboxylate residue appears at least once per unit in the chain. The repeating unit may be a monosaccharide or a disaccharide. In an embodiment, the carboxylated polysaccharide comprises one carboxyl group per monosaccharide.

By non-ionic detergent, surfactants containing no charged group, we include but are not limited to Triton X-100, nonyl phenoxypolyethoxylethanol (NP)-40, Tween-20, Tween-80, digitonin.

By ionic detergent, detergents have a hydrophilic head group that is charged and can be either negatively (anionic) or positively (cationic) charged, we include but are not limited to sodium dodecyl sulfate (SDS), sarkosyl, sodium deoxycholate.

By zwitter-ionic detergent we include 3-((3-cholamidopropyl) dimethylammonio)-1-propanesulfonate (CHAPS).

Chaotropic agents, that have the ability to disrupt hydrogen bonding and other non-covalent interactions between molecules, such as guanidinium thiocyanate, sodium iodide, and guanidinium hydrochloride, may also act as lysis agent and may replace detergent.

By “Triton X-100” we include Triton X-100 (C14H22O(C2H4O)n) is a nonionic surfactant that has a hydrophilic polyethylene oxide chain (on average it has 9.5 ethylene oxide units) and an aromatic hydrocarbon lipophilic or hydrophobic group. The hydrocarbon group is a 4-(1,1,3,3-tetramethylbutyl)-phenyl group.

In an embodiment, the agent is selected from the group consisting of: polyvinyl sulfonic acid (PVSA), vinyl sulfonic acid (VSA), heparin, sodium alginate, dextran sulfate, fucoidan, 2-(N-morpholino)ethanesulfonic acid (MES), sulphated cellulose, sulphated amylose, sulphated pectic acid, sulphated polyvinyl alcohol.

In an embodiment, the agent inhibits RNase. By “inhibit” in the context of the activity of Rnase, we include the meaning that the activity of at least one RNase is reduced in a sample to which an agent of the invention is added, compared to the activity in an analogous sample to which the agent is not added. Inhibition is not limited to complete inhibition or inactivation of a given RNase. In a given application, it may be that some low level of RNase activity can be tolerated that will not have a detrimental effect on the outcome of the reaction, purification and/or assay being performed (in this case, preparation of a cDNA sequencing library). In an embodiment, the agent inhibits the activity of an RNase by at least 10%, such as at least 20%, 30%, 40% or 50% compared to the activity in an analogous sample to which the agent is not added. In an embodiment, the agent inhibits the activity of an RNase by at least 50%, such as at least 60%, 70%, 80% or 90%, such as by 95% compared to the activity in an analogous sample to which the agent is not added. It will be appreciated that the extent to which the polymer inhibits the activity of RNase depends on the concentration of the polymer, the RNase concentration and the conditions of the reaction.

“Substantial inhibition” is achieved when the RNase activity in a sample is below the level that is tolerable in a given application (i.e., the preparation of a cDNA sequencing library, or other applications where inhibition of RNase activity is desired). The level of inhibition that is substantial will then depend upon the application in which the inhibitory agents are employed. In contrast, the term inactivation is used when there is no detectable level of activity of a given RNase. An RNase that is inactivated need not be rendered irreversibly inoperative. Agents of this invention may exhibit inhibition of certain RNases and inactivation of other RNases.

Examples of RNases that can be inactivated, inhibited and/or removed using the agent described herein include eukaryotic RNases (e.g., mammalian RNases or fungal RNases) and prokaryotic RNases. Specifically exemplary RNases include RNase A, RNase B, RNase C, RNase 1, RNase T1, and bacterial RNase (e.g., those of E. coli).

In an embodiment, the agent reduces and/or prevents RNA degradation. By “reduces and/or prevents RNA degradation” we include the meaning that the degradation of RNA is reduced in a sample to which an agent of the invention is added, compared to the degradation of RNA in an analogous sample to which the agent is not added. In an embodiment, the agent reduces the degradation of RNA by at least 10%, such as at least 20%, 30%, 40% or 50% compared to the degradation of RNA in an analogous sample to which the agent is not added. In an embodiment, the agent reduces the degradation of RNA by at least 50%, such as at least 60%, 70%, 80% or 90%, such as by 95% compared to the degradation of RNA in an analogous sample to which the agent is not added.

The agents of the invention can be used alone or in combination with other agents of the invention in the methods described herein.

In an embodiment, the RNase is selected from the group comprising RNase A and/or B; and/or C; and/or E. coli RNase. Preferably the agent (i.e. RNase inhibitor) does not inhibit or affect fidelity or processivity of modifying enzymes like reverse transcriptases, DNA polymerases, and/or transposases under the given reaction conditions.

Inactivation and/or inhibition is carried out by contacting a biological medium which may contain RNase with one or more agents as described herein. By “biological medium” we include the meaning of any liquid in which a biological reaction or assay can be carried out or performed during the preparation of a cDNA library, which might be detrimentally affected by the presence of one or more active RNases. Biological medium includes any buffers (e.g. storage and lysis buffers) and reagents employed in the preparation of a cDNA sequencing library. The inhibitory agents described herein can, for example, be added along with reagents (e.g., prior to, or simultaneous with reagents) to inactivate or inhibit RNases that might be present in a reaction mixtures. The inhibitory agents can be bound to the internal surfaces (e.g., glass, plastic, or fiber material) of containers, surfaces, or other equipment (e.g., multi-well plates, etc.) in which biological media, including buffers, are stored or in which biological purifications, reactions and or assays are carried out during the process of preparing a cDNA sequencing library. The inhibitory agents can also be bound in a material such as a membrane, for example a cotton or paper sheet pre-soaked in the RNase inhibitory agent, onto which a biological sample is added for storage and subsequent elusion and processing into an RNA-sequencing library.

Preparation of agent-coated surfaces can be achieved using an in situ polymerization method or by incubating material or surfaces with the inhibitory agent Those of ordinary skill in the art will appreciate that other means for directly or indirectly (through a linker) coupling of agents of this invention to surfaces are available in the art and can be employed in the practice of this invention.

By “the method includes the use of an agent”, we include the meaning that the agent is incorporated into a method of generating a cDNA library in such a way that RNase is inhibited. For example, the agent may already be included in a buffer that is used in the method or the agent may be added to one of the buffers used in the method before the method is carried out. The agent may be present in a reaction vessel (such as a multi-well plate) prior to be method being carried out. In an embodiment, the method includes the addition of the agent. In an embodiment, the method includes the use and/or addition of an effective amount of the agent. By “effective amount” we include the meaning of the amount of an agent or the combined amount of a mixture of agents which is used in or added to a biological medium containing one or more RNases to observe inhibition (as defined above) of at least one of the one or more RNases, whilst not substantially interfering with the biological reactions necessary for the generation of a cDNA sequencing library (e.g. first strand synthesis reaction and/or subsequent PCR reactions).

By “not substantially interfering” we include that addition of the agent does not negatively affect the biochemical reactions necessary for the generation of a cDNA sequencing library (e.g. first strand synthesis reaction and/or subsequent PCR reactions) such that the final yield of cDNA is not substantially decreased and that original RNA molecules are accurately recorded in the resulting sequencing library. This can be measured as the number of genes detected in a samples and the proportion of reads mapping to specific regions of the genome (exonic, intronic, intergenic). “Not substantially interfering” also includes polymerase processivity along the length of RNA transcripts and accuracy of nucleotide-sequence replication during RT and PCR during generation of the sequencing library, so that markedly increased frequency of incorrectly inserted bases does not occur. It will be appreciated that some level of decrease can be tolerated.

The amounts or combined amounts of agents of this invention that are inhibitory toward a given RNase or mixture of RNases or which render one or more RNases inactive can be readily determined by one of ordinary skill in the art without undue experimentation in view of the teachings herein and in view of what is generally known in the art.

For example, the purity and/or yield of RNA and cDNA retrieved in the presence of the agent can be measured using a spectrophotometer, fluorometer or Bioanalyzer/Fragment Analyzer and compared to the purity and/or yield of RNA retrieved in the absence of the agent. The quality of a total RNA prep can be assessed for signs of degradation by running a portion on an agarose or acrylamide gel or by using an instrument such as the Agilent Bioanalyzer. Examples of methods for assessing the quantity of RNA include using: UV absorbance, fluorescence, and an Agilent Bioanalyzer. RNA and resulting cDNA can be analysed by fluorometry for quantification and the Bioanalyzer (or equivalent device) for quantification and RNA integrity evaluation. A fluorometer (such as Life Technologies' Qubit) measures the concentration of RNA or DNA bound to a fluorescent dye. The concentration of RNA and cDNA can also be estimated from a Bioanalyzer or Fragment Analyzer trace. Another way to quantitate RNA is by measuring the absorbance at 260 nm. In case of full-length cDNA libraries, the size distribution of yielded cDNA after reverse transcription and PCR amplification provides an accurate readout of the underlying RNA integrity. Mammalian mRNA and full-length cDNA samples should display a characteristic peak at approximately 2000 bp, reflecting the length distribution of mRNAs in mammalian cells. Degradation, due to failed RNase inhibition, display an mRNA and cDNA size distribution skewed towards shorter fragments.

The agents of this invention can be used in combination or can be combined with any art-known RNase inhibitor (that are not agents of this invention) to achieve a desired inhibitory effect on or inactivation of one or more RNases.

It will be appreciated that the preparation of a cDNA library is the first step in a method of RNA sequencing (RNA-seq). By “RNA sequencing” or “RNA-seq” we include the meaning of a genomic approach for the detection and quantitative analysis of RNA molecules in a biological sample by the readout of nucleotide sequences. RNA-seq is a multi-purpose methodology that is increasingly used in biological, biomedical and clinical settings. RNA-seq can for example be useful for studying cellular states and responses in vivo and in vitro by studying protein-encoding mRNA molecules as well as non-protein-coding RNAs (collectively termed the ‘transcriptome’). RNA-seq is also a useful methodology to detect foreign biological material or infection in a sample, such as that of an RNA virus or bacteria transcribing their nucleic acid. RNA-seq is furthermore a useful readout in various in vitro applications and synthetic biology utilizing RNA.

The method of the invention may be used for bulk RNA sequencing or single-cell RNA sequencing. By “bulk RNA sequencing” we include the meaning of the sequencing of RNA isolated from pools of cells, including tissues, blood, secretions, tissue sections etc. By “bulk RNA sequencing” we also include the meaning of the sequencing of RNA from pools of cells, including tissues, blood, secretions, tissue sections etc. By “single cell RNA sequencing” or “scRNAseq” we include the meaning of the sequencing of RNA isolated from an individual cell which allows comparison of the transcriptomes of individual cells. Single-cell RNA-seq methods can also be used to detect RNA of parts or sub-compartments of a cell. The performance of scRNAseq methods can be characterized using single cells (generally containing 10-30 pg of total RNA in case of mammalian cells) or low amounts of input RNA, such as 10-100 pg of total RNA from an pool of RNA extracted from multiple cells.

It will be appreciated that the methods of the present invention can be used as part of any known method for preparing a cDNA sequencing library. Such methods are known in the art, and include but are not limited to, Smart-seq (Ramsköld, D. et al. Nat. Biotechnol. 30, 777-782 (2012)); Smart-seq2 (Picelli, S. et al. Nat. Methods 10, 1096-1098 (2013)); Smart-seq3 (Hagemann-Jensen, M. et al., 2020 and WO2020136438A1); STRT-seq (Islam, S. et al. Nat. Protoc. 7, 813-828 (2012)); STRT-seq-2i (Hochgerner, H. et al. Sci. Rep. 7, 16327 (2017)); SCRB-seq (Soumillon, M., Cacchiarelli, D., Semrau, S., van Oudenaarden, A. & Mikkelsen, T. S. Preprint at bioRxiv https://doi.org/10.1101/003236 (2014)); mcSCRB-seq (Bagnoli, J. W. et al. Preprint at bioRxiv https://doi.org/10.1101/188367 (2017)); Quartz-seq (Sasagawa, Y. et al. Genome Biol. 14, R31 (2013)); Quartz-seq2 (Sasagawa, Y. et al. Genome Biol. 19, 29 (2018)); CEL-seq (Hashimshony, T., Wagner, F., Sher, N. & Yanai, I. Cell Rep. 2, 666-673 (2012)); CEL-seq2 (Hashimshony, T. et al. Genome Biol. 17, 77 (2016)); MARS-seq (Jaitin, D. A. et al. Science 343, 776-779 (2014)); Seq-Well (Gierahn, T. M. et al. Nat. Methods 14, 395-398 (2017)); or inDrops (Klein, A. M. et al. Cell 161, 1187-1201 (2015)); Drop-seq Macosko, E. Z. et al. Cell 161, 1202-1214 (2015)), all of which are incorporated by reference, in particular methods for preparing a cDNA library.

It will also be appreciated that the methods of the present invention can be used as part of any “multiomics” method, i.e. a method that combines preparing a cDNA sequencing library from one part or fraction of the sample material and measurement of another biological modality from another part or fraction of the same sample, such as for example a DNA or protein library. This also includes in situ RNA sequencing methods in which spatial information of tissues and cells are captured in parallel with cDNA sequencing library generation. Such methods are known in the art, and include but are not limited to, sci-CAR (Cao e al. 2018 Science; https://doi.org/10.1126/science.aau0730, Smart3-ATAC (Cheng et al. bioRxiv 2021; https://doi.org/10.1101/2021.12.02.470912), and “Spatial Transcriptomics” (Stahl et al. Science 2016 10.1126/science.aaf2403), all of which are incorporated by reference, in particular the methods for preparing a cDNA library.

By “in situ RNA-sequencing” we include the meaning of RNA-sequencing methods wherein the RNA sequencing is performed directly on an intact tissue section; as well as that when RNA from a tissue section is first transferred and bound to a surface covered with barcoded Oligo dT primers, forming a pattern of RNA molecules on the surface that reproduce the relative location of the RNA molecules in the tissue, before commencing cDNA synthesis by reverse transcription, such as “spatial transcriptomics” described in Stahl et al. Science 2016 10.1126/science.aaf2403.

Where reference is made herein to a method comprising two or more defined steps, the defined steps can be carried out in any order or simultaneously (except where the context excludes that possibility), and the method can include one or more other steps which are carried out before any of the defined steps, between two of the defined steps, or after all the defined steps (except where the context excludes that possibility). Moreover, the method can be paused following one or more steps and resumed at a later stage, if technically appropriate to do so.

By “cDNA sequencing library” (may also be termed “next generation sequencing (NGS) library”) we include the meaning of a collection of complementary DNA (cDNA) fragments, which together constitute some portion of the transcriptome of a single cell or a plurality of cells. The collection of cDNA fragments in the library include a partial or complete sequencing platform adapter sequence at their termini useful for sequencing using a sequencing platform of interest.

Once prepared, the cDNA sequencing library can be subject to a full-length transcript, or 3′/5′-end sequencing protocol. By a “full-length transcript sequencing protocol” we include the meaning of methods that generates sequencing-read coverage across most of the length of the RNA transcripts, such as for example in Smart-seq (Ramsköld, D. et al. Nat. Biotechnol. 30, 777-782 (2012)); Smart-seq2 (Picelli, S. et al. Nat. Methods 10, 1096-1098 (2013)); Smart-seq3 (Hagemann-Jensen, M. et al., 2020 and WO2020136438A1); and Smart-seq3xpress (Hagemann-Jensen, M. et al., Nat Biotechnol 40, 1452-1457 (2022)). By “3′/5′-end sequencing protocol” we include the meaning of methods that generates sequencing-read coverage in either 3′ or 5′ end of the RNA transcripts coverage across most of the length of the RNA transcripts, such as for example in STRT-seq-2i (Hochgerner, H. et al. Sci. Rep. 7, 16327 (2017)); SCRB-seq (Soumillon, M., Cacchiarelli, D., Semrau, S., van Oudenaarden, A. & Mikkelsen, T. S. Preprint at bioRxiv https://doi.org/10.1101/003236 (2014)).

By “sequencing platform adapter sequence” or “sequencing platform adapter construct” we include the meaning of a nucleic acid construct that includes at least a portion of a nucleic acid domain (e.g., a sequencing platform adapter nucleic acid sequence) utilized by a sequencing platform of interest.

Most of the current sequencing platforms use clonal amplification to create clusters of identical molecules that are tethered next to each other on a solid support. For the Illumina platform the clusters are attached to the surface of a flow-cell, while for the 454, IonTorrent, and SOLiD platforms the clusters are generated on beads using emulsion PCR. Regardless of the platform, two types of adapter sequence are generally required: (1) platform-dependent domains that are required for clonal amplification and attachment to the sequencing support; and (2) a sequencing primer binding domain for priming the sequencing reaction. In addition, several optional elements may be present, including sequence tags to allow for multiplexing (known as barcodes or indices), unique molecular identifiers (UMIs), and/or a second sequence-priming site to allow for sequencing of the insert from the other side (known as paired-end sequencing).

In certain aspects, a sequencing platform adapter sequence includes one or more nucleic acid domains selected from: a platform-dependent domain that specifically binds to a surface-attached sequencing platform oligonucleotide (e.g., the P5 or P7 oligonucleotides attached to the surface of a flow cell in an Illumina® sequencing system); a sequencing primer binding domain (e.g., a domain to which the Read 1 or Read 2 primers of the Illumina® platform may bind); a barcode domain (e.g., a domain that uniquely identifies the sample source of the nucleic acid being sequenced to enable sample multiplexing by marking every molecule from a given sample with a specific barcode or “tag”); a barcode sequencing primer binding domain (a domain to which a primer used for sequencing a barcode binds); a unique molecular identification domain (e.g., a molecular index tag, such as a randomized tag of 4, 6, or other number of nucleotides) for uniquely marking molecules of interest to determine expression levels based on the number of instances a unique tag is sequenced; or any combination of such domains.

In certain aspects, a barcode domain (e.g., sample index tag combination including a unique index or unique dual indexes (UDIs)) and a unique molecular identifier (UMI) domain (i.e., molecule index tag) may be included in the same nucleic acid domain. A sequencing platform adapter construct, when present, may include one or more nucleic acid domains of any length and sequence suitable for the sequencing platform of interest. In certain aspects, the nucleic acid domains are from 4 to 200 nts in length. For example, the nucleic acid domains may be from 4 to 100 nts in length, such as from 6 to 75, from 8 to 50, or from 10 to 40 nts in length. The sequencing platform adapter construct may include a nucleic acid domain that is from 2 to 8 nucleotides in length, such as from 9 to 15, from 16 to 22, from 23 to 29, or from 30 to 36 nts in length.

Such sequencing platform adapter constructs can be added to each end of the insert during the first- and/or second-strand synthesis steps. In this case the reverse transcriptase primer can contain an overhanging or nested sequence that does not anneal to the RNA template but contains at least a portion of the adapter sequences. In a similar manner the forward PCR primer can contain over-hanging sequences and therefore introduce such adapters. Alternatively, adapters can be introduced via ligation. This approach is used in the Illumina TruSeq Small RNA kit, the NEBNext Small RNA prep kit, and in the SOLiD RNA kits from Life Technologies. These kits use ligation procedures that allow two different adapters to be ligated onto each end of the target RNA. These adapters are then used to prime the first- and second-strand synthesis reactions resulting in cDNAs terminated by the appropriate adapter sequences.

It will be appreciated that the nucleotide sequences of nucleic acid domains useful for sequencing on a sequencing platform of interest may vary and/or change over time. Adapter sequences are typically provided by the manufacturer of the sequencing platform (e.g., in technical documents provided with the sequencing system and/or available on the manufacturer's website). Based on such information, the sequence of any sequencing platform adapter domains, such as the template switch oligonucleotide, first strand cDNA primer, amplification primers, and/or the like, may be designed to include all or a portion of one or more nucleic acid domains in a configuration that enables sequencing the nucleic acid insert (corresponding to the template RNA) on the platform of interest.

By “sequencing” we include the meaning of high throughput sequencing. By “high throughput sequencing” we include the meaning of the simultaneous or near simultaneous sequencing of thousands of nucleic acid molecules. High throughput sequencing is sometimes referred to as “next generation sequencing (NGS)” or “massively parallel sequencing”.

Sequencing platforms of interest include, but are not limited to, sequencing platforms provided by Illumina® (e.g., the NextSeq™, HiSeq™, MiSeq™, NovaSeq™ and/or Genome Analyzer™ sequencing systems); Ion Torrent™ (e.g., the Ion PGM™ and/or Ion Proton™ sequencing systems); Pacific Biosciences (e.g., the PACBIO RS II sequencing system); Life Technologies™ (e.g., a SOLiD sequencing system); Roche (e.g., the 454 GS FLX+ and/or GS Junior sequencing systems); MGI Tech Co., Ltd. “MGI” (e.g., the DNBSEQ-T7™, DNBSEQ-G400™, DNBSEQ-G50™) or any other sequencing platform of interest.

In a particular embodiment, the method comprises:
i. releasing a plurality of RNA molecules from one or more cells or cell extract in the presence of an aqueous solution comprising the agent, wherein the agent is according to any embodiment disclosed herein;
ii. synthesizing a plurality of cDNA strands from the RNA molecules by reverse transcription; and
iii. processing the cDNA strands to generate a cDNA sequencing library.

Releasing a plurality of RNA molecules from one or more cells or cell extract can be achieved by, for example, heating or freeze-thaw of cells, or by the use of detergents, chaotropic agents, mechanical methods, or other chemical methods, or by a combination of these, in the presence of an aqueous solution comprising the agent. Mechanical methods for homogenizing tissues include using cryo-grinding with a mortar/pestle, shearing using a rotor-stator homogenizer or a Dounce homogenizer, sonication, or bead-beating. After homogenization two methods are commonly used to recover RNA from the cell lysate: (1) extraction with organic solvents; or (2) solid-phase extraction on silica.

In an embodiment, releasing a plurality of RNA molecules from one or more cells or cell extract in the presence of an aqueous solution comprising the agent comprises contacting one or more cells or cell extract with an aqueous solution to release RNA molecules. The RNA molecules are preferably poly(A) containing RNA molecules, such as mRNA molecules, and are typically present in and released from the cytoplasm of the lysed cell.

By “aqueous solution” we include the meaning of any liquid solution that can be used in a method of liberating RNA from cells. Such aqueous solutions include buffers such as sample collection buffers and lysis buffers. Examples of suitable buffers include, PBS, Tris, sodium-acetate, HEPES, MOPS. In an embodiment, the aqueous solution may be a sample collection buffer. A sample collection buffer may not comprise a detergent and/or chaotropic agent. For example, a sample collection buffer could contain the bulk sample of intact cells without detergent, and the RNA can be extracted through another means, such as using Trizol, phenol, and/or a commercially available RNA extraction kit. In an embodiment, the aqueous solution may be a lysis buffer.

By “lysis buffer” we include the meaning of a buffer used for the purpose of breaking open cells. Examples of suitable lysis buffer to which the agent could be added are described herein and are described in known protocols for preparing a cDNA library. For example, the lysis buffer may comprise enzymes (e.g. Proteinase K), detergents (e.g. Triton X-100, SDS, NP-40/Igepal, Tween-20, sodium deoxycholate, and CHAPS) and/or chaotropic agent (i.e. (compounds that disrupt both hydrophobic and hydrogen-bond interactions, such as guanidine salts) together with the agent. For instance, Triton X-100 could be used as a detergent when lysing cells. Guanidinium is a strong protein denaturant capable of denaturing recalcitrant proteins such as RNases. In an embodiment, the buffer is a lysis buffer comprising 0.1-1% Triton X-100 and the agent. In an embodiment the buffer is a lysis buffer comprising 0.1% Triton X-100. A mild lysis procedure can advantageously be used to prevent the release of nuclear chromatin, thereby avoiding genomic contamination of the cDNA library, and to minimize degradation of mRNA. For example, heating the cells at 72° C. for 2-10 minutes in the presence of mild detergent (together with the agent) is generally sufficient to lyse cells.

In some embodiments, after release of RNA molecules, specific classes of RNA can be enriched in the sample to be sequenced. Total RNA recovered using standard cell lysis procedures described above typically consists of >80% ribosomal RNA (rRNA), so if rRNA were not removed, the majority of the final sequence reads would be from rRNA. There are several methods known in the art for performing this step.

It is possible to use an oligo-dT to selectively recover mature mRNAs by duplexing with their poly-A tails (discussed below). Several variants of this method have been developed, which include using columns containing oligo-dT bound to cellulose, using oligo-dT bound to plastic plates via a biotin linkage, and using magnetic beads to which oligo-dT has been attached via a biotin linkage. All of these approaches work well and numerous commercial kits are available.

In scRNA-seq library preparation, no purification of mRNA is generally performed. Instead the mRNA is selectively reverse transcribed into cDNA using oligo-dT primers. These oligo-dT primers also contain a nested primer sequence located 5′ of the oligo-dT part that can be used for amplification in a following PCR reaction. In an embodiment, ribosomal RNA is not removed during library preparation.

Alternatively, SuperSAGE, a high-throughput version of SAGE (serial analysis of gene expression), is an approach that targets for sequencing just the 3′-end of transcripts with a polyA tail. Biotinylated oligo-dTs that include an EcoP15I recognition site are hybridized to polyadenylated mRNAs in the sample, followed by first- and second-strand cDNA synthesis. The cDNA is then cut upstream of the polyA tail with a frequent-cutting restriction enzyme such as NlaIII, and pulled down with streptavidin-coated beads. At this point an adapter, which includes the platform-specific nucleotides necessary for high-throughput sequencing, another EcoP15I recognition site, and a NlaIII overhang is ligated to the bead-bound tags. EcoP15I digestion will then cut the cDNA at a distance 25-27 nt from the recognition sequence, and this portion of the tag is sequenced after ligation of another adapter with platform-specific nucleotides, and PCR amplification (Methods Mol Biol. 2012; 883:1-17. doi: 10.1007/978-1-61779-839-9_1.).

Alternatively, several kits can be used to selectively remove ribosomal RNA from total RNA samples. In general, the oligo/rRNA complex is removed from the solution via binding to beads. Different kits use different technologies to capture the bound complex. For example, the capture oligos in the Ribominus (Invitrogen/Life Technologies) and Ribo-Zero (Epicentre/Illumina) kits have a biotin tag, that can be captured using streptavidin coated magnetic beads. The GeneRead kit (Qiagen) uses antibodies that specifically recognize the rRNA/oligo complex.

Most current sequencing platforms are capable of providing only relatively short sequence reads (˜40-400 bp depending upon the platform). Therefore, most RNA-seq protocols incorporate a fragmentation step to improve sequence coverage over the transcriptome. However, protocols differ as to when the fragmentation is performed. Most of the original protocols fragmented the cDNA; however, fragmentation of the RNA (before converting it to cDNA) is also possible. Methods used to fragment RNA include: enzymatic, metal ion, heat, and sonication. The goal is to produce a population of RNA fragments that are of desired length for the following sequencing methodology, often on average about 200 bases.

The term “one or more cells” refers to any number of (e.g. unlysed) cells desired to be analysed. One or more cells may include at least 1 cell, at least 10 cells, or alternatively at least 25 cells, or alternatively at least 50 cells, or alternatively at least 100 cells, or alternatively at least 200 cells, or alternatively at least 500 cells, or alternatively at least 1000 cells, or alternatively 5,000 cells or alternatively 10,000 cells. One or more cells may include from 10 to 100 cells, or alternatively from 50 to 200 cells, alternatively from 100 to 500 cells, or alternatively from 100 to 1000, or alternatively from 1,000 to 5,000 cells. One or more cells may include 10,000 cells, 20,000 cells, 30,000 cells, 40,000 cells, 50,000 cells, 60,000 cells, 70,000 cells, 80,000 cells, 90,000 cells or alternatively 100,000 cells.

By “cell extract” we include a preparation obtained by breaking open cells, which preparation may contain some or all of the soluble molecules of a cell, and in which the integrity of RNA is maintained. Methods for preparing such cell extracts are known in the art, and include density gradient methods, physical disruption (e.g. homogenization) and chemical disruption. In some embodiments, the cell extract may comprise RNA from cells. As shown in the accompanying Examples, a cell extract comprising bulk RNA was obtained from cultured mouse tail tip fibroblasts using TRIzol (Invitrogen). 100 pg of RNA was then added to PCR tubes containing a lysis buffer comprising the agent.

The “one or more cells or cell extract” comprises template RNA and may be derived from any sample of interest, including but not limited to, a single cell, a plurality of cells (e.g., cultured cells), a tissue, an organ, or an organism (e.g., bacteria, yeast, or higher eukaryotic organisms, such as a plant, or a mouse, or a worm, or the like). In certain embodiments, the one or more cells or cell extract are derived from a tissue, organ, and/or the like of a mammal (e.g., a human, a rodent (e.g., a mouse), or any other mammal of interest). The one or more cells or cell extract can be derived from live samples, non-conserved samples, preserved samples, embalmed samples and/or fixed samples. In certain aspects, the RNA molecules are liberated into an aqueous solution comprising the agent from one or more cells in a fixed biological sample, e.g., formalin-fixed, formaldehyde/paraformaldehyde-fixed, paraffin-embedded (FFPE) tissue. RNA from one or more cells in FFPE tissue may be released using commercially available kits—such as the NucleoSpin® FFPE RNA kits by Clontech Laboratories, Inc. (Mountain View, CA).

Further non-limiting examples for samples from which one or more cells or cell extract can be derived from includes a cell culture sample, blood, serum, plasma, reticulocytes, lymphocytes, any product prepared from blood or lymph, bone marrow tissue, cerebrospinal fluid, sweat, tear, saliva, sputum, amniotic fluid, seminal fluid, vaginal excretion, serous fluid, synovial fluid, pericardial fluid, peritoneal fluid, pleural fluid, transudates, exudates, cystic fluid, bile, urine, gastric fluid, intestinal fluid, or faecal samples), any type of tissue biopsy (e.g. a tumour biopsy, a muscle biopsy, a liver biopsy, a kidney biopsy, a bladder biopsy, a bone biopsy, a cartilage biopsy, a skin biopsy, a pancreas biopsy, a biopsy of the intestinal tract, a thymus biopsy, a uterus biopsy, a testicular biopsy, an eye biopsy or a brain biopsy), or any other biological material that may harbor RNA molecules. Suitable samples containing cells further comprise clinical samples (which are samples provided by a patient), biological swabs and biological washes. Suitable samples containing cells may be fresh or may have been stored (e.g. cryopreserved), such as at −80° C. Furthermore, in general, cells from any population can be used in the methods, such as a population of prokaryotic or eukaryotic single celled organisms including bacteria or yeast.

After obtaining an RNA preparation that is suitable for RNA-seq (step 1) the RNA is typically converted to double-stranded complementary DNA (cDNA). Currently available sequencing technologies require a DNA template with platform-specific “adaptor” sequences at either end of each molecule, as discussed in detail herein. Generating the cDNA, adding the adaptors, and (if necessary) amplifying the DNA for sequencing encompasses steps (ii) and/or (iii) of the method described herein.

In order to convert RNA to DNA the RNA must be used as a template for a DNA polymerase. Most DNA polymerases cannot use RNA as a template. However, retroviruses encode a unique type of polymerase known as reverse transcriptases, which are able to synthesize DNA using an RNA template.

Reverse transcriptase, like other polymerases, requires a primer annealed to either DNA or RNA to initiate polymerization. Several first-strand priming options can be used in the generation of a cDNA sequencing library and are discussed in more detail herein.

The inventors have found that the agents of the invention remain stable throughout thermocycles and therefore frequent addition of the inhibitory agent is not required. In an embodiment, synthesis of cDNA strands from RNA is performed directly on cell lysates, such that a reaction mix for reverse transcription (first strand synthesis) is added directly to cell lysates which contain the agent. Accordingly, the cells are lysed and reverse transcription reaction mix is added directly to the lysates without additional purification, therefore the agent will be present in the reverse transcription reaction mix (albeit at a more dilute concentration). Accordingly, it will be appreciated that the invention provides a method of preparing a cDNA sequencing library in which the inhibitory agent is provided only once during the course of the method, for example prior to or during the cell lysis stage. Thus, the invention also includes a method of preparing a cDNA sequencing library in which an RNase inhibitor (e.g. an agent of the invention or any known RNase inhibitor) is not added after the cell lysis stage.

Alternatively, RNA, such as mRNA, can be purified after its release from cells, as discussed herein. Alternatively, specific contaminants, such as ribosomal RNA can be selectively removed, as discussed herein.

By “reverse transcription” we include the meaning of a process whereby an RNA-dependent DNA polymerase having reverse transcriptase activity extends an oligonucleotide primer hybridized to an RNA template, in the presence of deoxynucleoside 5′-triphosphates (dNTPs), whether natural or modified, resulting in synthesis of complementary DNA (cDNA). Methods for synthesizing cDNA from small amounts of RNA, including from single cells, have been described before (Picelli, S. et al. Nat. Methods 10, 1096-1098 (2013), Hagemann-Jensen, M. et al., (2020), Hochgerner, H. et al. Sci. Rep. 7, 16327 (2017), Soumillon, M., Cacchiarelli, D., Semrau, S., van Oudenaarden, A. & Mikkelsen, T. S. Preprint at bioRxiv https://doi.org/10.1101/003236 (2014), Bagnoli, J. W. et al. Preprint at bioRxiv https://doi.org/10.1101/188367 (2017), Sasagawa, Y. et al. Genome Biol. 19, 29 (2018), Hashimshony, T. et al. Genome Biol. 17, 77 (2016), Gierahn, T. M. et al. Nat. Methods 14, 395-398 (2017), Klein, A. M. et al. Cell 161, 1187-1201 (2015), Macosko, E. Z. et al. Cell 161, 1202-1214 (2015).

Reverse transcription is well known in the art and can be carried out using a reverse transcription primer comprising a recognition sequence, complementary to a sequence in the target deoxyribonucleic and/or ribonucleic acid sequence. The reverse transcription primer can be used as an anchored primer in a reverse transcription reaction to generate a primer extension product, complementary to the target RNA sequence using a reverse transcriptase enzyme.

By synthesizing a “plurality” of cDNA strands we include the meaning of the synthesis of any number of cDNA strands desired to be analyzed. In some aspects of the invention, a plurality of cDNA strands includes at least 10 cDNA strands, or alternatively at least 25 cDNA strands, or alternatively at least 50 cells, or alternatively at least 100 cDNA strands, or alternatively at least 200 cDNA strands, or alternatively at least 500 cDNA strands, or alternatively at least 1000 cDNA strands, or alternatively 5,000 cDNA strands or alternatively 10,000 cDNA strands. A plurality of cDNA strands may include from 10 to 100 cDNA strands, or alternatively from 50 to 200 cDNA strands, alternatively from 100 to 500 cDNA strands, or alternatively from 100 to 1000, or alternatively from 1,000 to 5,000 cDNA strands. A plurality of cDNA strands may include 50,000, 100,000, 200,000, 300,000, 400,000, 500,000, 600,000, 700,000, 800,000, 900,000 or alternatively at least 1,000000 strands.

By “RNA molecules” we include the meaning of the template ribonucleic acid (RNA) liberated from the one or more cells or contained within the cell extract. It may be a polymer of any length composed of ribonucleotides, e.g., 10 nts or longer, 20 nts or longer, 50 nts or longer, 100 nts or longer, 500 nts or longer, 1000 nts or longer, 2000 nts or longer, 3000 nts or longer, 4000 nts or longer, 5000 nts or longer or more nts. In certain aspects, the template ribonucleic acid (RNA) is a polymer composed of ribonucleotides, e.g., 10 nts or less, 20 nts or less, 50 nts or less, 100 nts or less, 500 nts or less, 1000 nts or less, 2000 nts or less, 3000 nts or less, 4000 nts or less, or 5000 nts or less, 10,000 nts or less, 25,000 nts or less, 50,000 nts or less, 75,000 nts or less, 100,000 nts or less. The template RNA may be any type of RNA (or sub-type thereof) including, but not limited to, a messenger RNA (mRNA), a microRNA (miRNA), a small interfering RNA (siRNA), a transacting small interfering RNA (ta-siRNA), a natural small interfering RNA (nat-siRNA), a ribosomal RNA (rRNA), a transfer RNA (tRNA), a small nucleolar RNA (snoRNA), a small nuclear RNA (snRNA), a long non-coding RNA (lncRNA), a non-coding RNA (ncRNA), a transfer-messenger RNA (tmRNA), a precursor messenger RNA (pre-mRNA), or any combination of RNA types thereof or subtypes thereof.

By “processing the cDNA strands to generate a cDNA sequencing library” we include the meaning of any subsequent step(s) necessary to convert newly synthesised cDNA strands into a cDNA library that is suitable for sequencing (i.e. a cDNA sequencing library). Such steps may include the generation of a second complementary strand, further amplification steps in order to increase the amount of cDNA, further steps which introduce additional tags into the cDNAs (such as sequencing adapter constructs), such steps may include the fragmentation of the cDNA strands. For example, the cDNA generated in step (ii) may be amplified using two primers in a PCR reaction and the amplified product may be fragmented using, for instance, ILLUMINA® Nextera XT kit to be prepared for sequencing by ILLUMINA® platforms.

In a particular embodiment, step (ii) comprises hybridizing a first strand cDNA synthesis primer to an RNA molecule and synthesizing a first cDNA strand complementary to at least a portion of the RNA molecule by reverse transcription.

In an embodiment, a cDNA synthesis primer binds to an RNA molecule to generate a respective cDNA strand complementary to at least a portion of the RNA molecule to form a respective RNA-cDNA intermediate. It will be appreciated that this step is performed in the presence of a reverse transcriptase enzyme. This step generates an unamplified first strand cDNA template in the form of a single-stranded cDNA or an RNA-cDNA intermediate.

By “hybridizing”, “hybridization,” or “annealing” we include the meaning of a process of interaction between two or more polynucleotides forming a complementary complex through base pairing which is most commonly a duplex or double-stranded complex.

By “oligonucleotide”, “primer”, or “oligonucleotide primer” we include the meaning of a single-stranded multimer of nucleotides from 2 to 500 nts, generally with a free 3′-OH group that binds to a target or template potentially present in a sample of interest by hybridizing with the target, and thereafter promoting polymerization of a poly nucleotide complementary to the target. An oligonucleotide can comprise modified nucleotides, such as methylated nucleotides and nucleotide analogues. Primers may be synthetic or may be made enzymatically, and, in some embodiments, are 10 to 50 nucleotides in length, or alternatively are 20-80 nucleotides in length. Primers for use in the present methods generally comprise of nucleotides ranging from 17 to 30 nucleotides. The primers may be 17 nucleotides, or alternatively, 18 nucleotides, or alternatively, 19 nucleotides, or alternatively, 20 nucleotides, or alternatively, 21 nucleotides, or alternatively, 22 nucleotides, or alternatively, 23 nucleotides, or alternatively, 24 nucleotides, or alternatively, 25 nucleotides, or alternatively, 26 nucleotides, or alternatively, 27 nucleotides, or alternatively, 28 nucleotides, or alternatively, 29 nucleotides, or alternatively, 30 nucleotides, 40 nucleotides, or alternatively 50 nucleotides, or 60 nucleotides, alternatively 70 nucleotides, 80 nucleotides, 90 nucleotides, or alternatively 100 nucleotides.

In some aspects of the invention, the synthesis of the first strand of cDNA from RNA can be directed by a “first strand cDNA synthesis primer” that includes an RNA complementary sequence. The RNA complementary sequence is at least partially complementary to one or more mRNA in an individual mRNA sample. This allows the primer, which is typically an oligonucleotide, to hybridize to at least some mRNA in an individual mRNA sample to direct cDNA synthesis using the mRNA as a template. The RNA complementary sequence can comprise oligo (dT), or be gene family-specific, such as a sequence of nucleic acids present in all or a majority related genes, or can be composed of a random sequence, such as random hexamers. To avoid the cDNA synthesis primer priming on itself and thus generating undesired side products, a non-self-complementary semi-random sequence can be used. For example, one letter of the genetic code can be excluded, or a more complex design can be used while restricting the cDNA synthesis primer to be non-self-complementary.

By “complementary” we include the meaning of a nucleotide sequence that base-pairs by non-covalent bonds to all or a region of a target nucleic acid (e.g., a template RNA or other region of the double stranded product nucleic acid). In the canonical Watson-Crick base pairing, adenine (A) forms a base pair with thymine (T), and guanine (G) pairs with cytosine (C) in DNA. In RNA, thymine is replaced by uracil (U). As such, A is complementary to T and G is complementary to C. However, in RNA, A is complementary to U and vice versa. Generally, “complementary” refers to a nucleotide sequence that is at least partially complementary. The term “complementary” may also encompass duplexes that are fully complementary such that every nucleotide in one strand is complementary to every nucleotide in the other strand in corresponding positions. In certain cases, a nucleotide sequence may be partially complementary to a target, in which not all nucleotides are complementary to every nucleotide in the target nucleic acid in all the corresponding positions.

An RNase (e.g. an enzyme having RNaseH activity) can be added after synthesis of the first strand of cDNA to degrade the RNA strand and to permit synthesis of a second strand of cDNA.

In a particular embodiment, synthesizing a first cDNA strand comprises the use of a first strand synthesis primer selected from: an oligo-dT primer, a primer with a random sequence, a degenerate primer specific to a gene family, a gene specific primer, and/or a primer complementary to a pre-ligated oligo.

In an embodiment, the first strand synthesis primer is an oligo-dT primer. It will be appreciated that first strand priming may comprise an oligo-dT primer to prime synthesis off of the poly-A tail that is found on most mature eukaryotic mRNAs. This method has the advantage that since the priming sequence is the same for all mRNAs then they should all be equally primed regardless of their coding sequence.

An oligo-dT primer, preferably an anchored oligo-dT primer, is complementary to and capable of hybridizing to a poly-A tail of the RNA molecule. In the case of an anchored oligo-dT primer, the oligo-dT primer comprises at least one additional selective nucleotide. As is well known in the art, a eukaryotic mRNA typically contains, from a 5′-end to a 3′-end, a cap, a 5′ untranslated region (UTR), the coding sequence (CDS), a 3′ UTR and the poly-A tail. This means that the anchored oligo-dT primer preferably comprises at least one nucleotide that is complementary to the last nucleotide(s) in the 3′ UTR or, in the case the mRNA molecule lacks a 3′ UTR, to the last nucleotide(s) in the CDR, in addition to the poly-A tail. Oligo-dT primers are known in the art, for example (Picelli, 2014 https://www.nature.com/articles/nprot.2014.006) and described in the accompanying Examples.

In an embodiment, the first strand synthesis primer is a primer with a random sequence. It will be appreciated that first strand synthesis can be primed using primers with random sequences. This approach means that non-polyadenylated RNAs will be recovered, making it possible to recover ncRNAs and use RNA fragmentation.

As used herein, the terms “primer with a random sequence” and “random primer” are used interchangeably.

Random primers can exhibit four-fold degeneracy at each position. The random primer may comprise nucleic acid primers that are any of a variety of random sequence lengths, as known in the art. For example, the random primer can comprise a random sequence that is 3, 4, 5, 6, 7, 8, 10, 11, 12, 13, 14, 15, 16, 17, 18, 19, 20 or more nucleotides long.

A plurality of random primers can comprise random primers of various lengths. A plurality of random primers can comprise randomers that are of equal length. A plurality of random primers can comprise a random sequence that is about 5 to about 18 nucleotides long. In some embodiments, the plurality of random primers comprises random hexamers. Random primers, and particularly random hexamers, are commercially available and widely used in amplification reactions such as Multiple Displacement Amplification (MDA), as exemplified by REPLI-g whole genome amplification kits (QIAGEN, Valencia, Calif.). It will be appreciated that any suitable length of random primer may be used in the methods and compositions presented herein.

In an embodiment, the first strand synthesis primer is a primer complementary to a pre-ligated oligo. It will be appreciated that another option for first strand synthesis is to first ligate an adapter with a known sequence to the 3′-end of the template RNA molecule using T4-RNA ligase. This sequence can subsequently be used to prime synthesis of the first strand.

In an embodiment, the first strand synthesis primer is a degenerate primer specific to a gene family. A primer, or more generally any DNA sequence, is called specific if it represents a unique sequence and is called degenerate if it represents a collection of unique sequences. Degenerate primers based on the amino acid sequence of conserved regions can be used for members of a gene family.

In an embodiment, the first strand synthesis primer is a gene specific primer. By “gene-specific primer” we include the meaning of a primer that enables the detection of specific genes.

In a particular embodiment, the first strand cDNA synthesis primer comprises a tag, such that synthesizing the first cDNA strand incorporates the tag into the cDNA to provide a tagged cDNA strand, and wherein the tag comprises a unique molecular identifier (UMI) sequence and/or a barcode.

By “barcode” we include the meaning of a nucleic acid tag that can be used to identify a sample or source of the nucleic acid material. Barcodes may vary, wherein examples include RNA source barcodes, e.g., cell barcodes, host barcodes; container barcodes, such as plate or well barcodes; in-line barcodes, indexing barcodes, etc. Therefore, where nucleic acid samples are derived from multiple sources, the nucleic acids in each nucleic acid sample can be tagged with different nucleic acid tags such that the source of the sample can be identified. Accordingly, the inclusion of a barcode at this stage in the preparation of a cDNA library for scRNA-seq allows early pooling of cells and cost-effective multiplex processing. By “multiplex processing” or “multiplexing” we include the meaning of pooling libraries from multiple experiments into a single sequencing reaction. Barcodes, also commonly referred to indexes, tags, etc are well known in the art. The tags are typically short (5-12 bp) sequences that are read during sequencing. Any suitable barcode or set of barcodes can be used, as known in the art and as exemplified by the disclosures of DOI: 10.1002/advs.202101229.

By “UMI”, “unique identifier”, and “unique molecular identifier” we include the meaning of a unique nucleic acid sequence that is attached to each of a plurality of nucleic acid molecules. When incorporated into a nucleic acid molecule, for example during first strand cDNA synthesis, a UMI can be used to correct for subsequent amplification bias by directly counting unique molecular identifiers (UMIs) that are sequenced after amplification. PCR bias introduced during cDNA library preparation can be reduced and a more quantitative understanding of the sample population can be achieved. The design, incorporation and application of UMIs can take place as known in the art, as exemplified by, for example, the disclosures of (doi.org/10.1186/s12864-018-4933-1).

In some embodiments, the tag comprises a barcode without the UMI. In some embodiments, the tag comprises a UMI without the barcode.

In some embodiments, the first strand synthesis primer comprises a nested primer position which allows for the amplification of cDNA after RT.

In some embodiments, the first strand synthesis primer comprises a PCR handle. By “PCR handle” we include the meaning of a stretch of nucleotides that can be used to amplify the first strand cDNA molecule into many copies that can be detected by sequencing.

In the embodiment in which a plurality of RNA molecules are released from one or more cells, in a further embodiment, the one or more cells are spatially separated into single cells prior to the release of RNA molecules such that a plurality of individual RNA samples is provided, and each individual RNA sample comprises a plurality of RNA molecules from a single cell.

By a “single cell(s)” we include the meaning of one cell. Single cells useful in the methods described herein can be obtained as discussed herein. A single cell suspension can be obtained using standard methods known in the art including, for example, enzymatically using trypsin or papain to digest proteins connecting cells in tissue samples or releasing adherent cells in culture, or mechanically separating cells in a sample.

By “spatially separated” we include the meaning that single cells are segregated into a spatial compartment. In other words, single cells can be placed in any suitable reaction vessel in which single cells can be treated individually. For example a single PCR tube, 8-well strip of PCR tubes, 96-well plate, 384 well plate, or a plate with any number of wells such as 2000, 4000, 6000, or 10000 or more. The multi-well plate can be part of a chip and/or device. The present disclosure is not limited by the number of wells in the multi-well plate in various embodiments, the total number of wells on the plate is from 96 to 200,000, or from 5,000 to 10,000. The plate may comprise smaller chips, each of which includes 5,000 to 20,000 wells. For example, a square chip may include 125 by 125 nanowells, with a diameter of 0.1 mm. The wells (e.g., nanowells) in the multi-well plates may be fabricated in any convenient size, shape or volume. The well may be 100 mm to 1 mm in length, 100 pm to 1 mm in width, and 100 pm to 1 mm in depth. Each nanowell may have an aspect ratio (ratio of depth to width) of from 1 to 4. The transverse sectional area may be circular, elliptical, oval, conical, rectangular, triangular, polyhedral, or in any other shape. The transverse area at any given depth of the well may also vary in size and shape. The wells may have a volume of from 0.1 nl to 1 mL. The nanowell may have a volume of 1 mL or less, such as 500 nl or less. The volume may be 200 nl or less, such as 100 nl or less. In an embodiment, the volume of the nanowell is 100 nl. Where desired, the nanowell can be fabricated to increase the surface area to volume ratio, thereby facilitating heat transfer through the unit, which can reduce the ramp time of a thermal cycle. The cavity of each well (e.g., nanowell) may take a variety of configurations. For instance, the cavity within a well may be divided by linear or curved walls to form separate but adjacent compartments, or by circular walls to form inner and outer annular compartments. The wells can be designed such that a single well includes a single cell. An individual cell may also be isolated in any other suitable container, e.g., microfluidic chamber, droplet, nanowell, tube, etc. Microfluidic systems capture cells in integrated fluidics circuits (IFCs), droplets or nanowells, thus allowing thousands of cells to be processed simultaneously while minimizing reaction volumes and reagent use.

Any convenient method for manipulating single cells may be employed, where such methods include fluorescence activated cell sorting (FACS), robotic device injection, gravity flow, or micromanipulation and the use of semi-automated cell pickers (e.g. the Quixell™ cell transfer system from Stoelting Co.), etc. FACS can be used to sort cells into microtiter plates ready for library preparation by manual or automated processing, and facilitates the exclusion of dead or damaged cells, as well as the enrichment of target cell populations (e.g., through surface marker labelling). To reduce background and maximize assay performance, FACS or magnetic-activated cell sorting (MACS) processing of single-cell solutions for microfluidic systems can be used to remove debris, damaged/dead cells and cell aggregates. In an embodiment, the cells are spatially separated by FACS and each cell is sorted into a spatial compartment, e.g., single microwell on a Fluidigm C1 chip. In some embodiments, each cell is spatially separated into a spatial compartment by being immobilized on a solid surface such as a flow cell or a bead.

In some instances, single cells can be deposited in wells of a plate according to Poisson statistics (e.g., such that approximately 10%, 20%, 30% or 40% or more of the wells contain a single cell (which number can be defined by adjusting the number of cells in a given unit volume of fluid that is to be dispensed into the containers)). In some instances, a suitable reaction vessel comprises a droplet (e.g., a microdroplet). Individual cells can, for example, be individually selected based on features detectable by microscopic observation, such as location, morphology, reporter gene expression, antibody labelling of DNA, RNA, or protein, DNA/RNA FISH, intracellular RNA labelling, or qPCR.

In an embodiment, one or more cells, or cell extract, is contacted with a lysis buffer comprising the agent and a single-cell suspension is obtained. A single cell is placed in one well of a multi-well plate, or other suitable vessel, such as a droplet, microfluidic chamber or tube. The cells are lysed and reverse transcription reaction mix is added directly to the lysates without additional purification. It is also possible that the container vessel also contains reagents necessary for reverse transcription when the cells are lysed.

In an embodiment there is provided a multiwell plate for single cell lysis wherein the plate wells contain a detergent, dNTP, PVSA, and primers for reverse transcription.

In such a multiwell plate the concentration of PVSA may be in the range of 0.1-120 μg/mL, such as from 0.3 to 110 μg/mL, such as from 0.5 to 100 μg/mL, such as from 1 to 90 μg/mL, such as from 2 to 80 μg/mL, such as from 5 to 70 μg/mL, such as from 10 to 60 μg/mL, such as from 15 to 50 μg/mL, such as from 20 to 40 μg/mL, such as 30 μg/mL. The detergent may be Triton X-100 or another suitable detergent.

In the embodiment in which the one or more cells are spatially separated into single cells prior to the release of RNA molecules such that a plurality of individual RNA samples is provided and each individual RNA sample comprises a plurality of RNA molecules from a single cell, in a further embodiment, the method comprises hybridizing a first strand cDNA synthesis primer to an RNA molecule and synthesizing a first cDNA strand complementary to at least a portion of the RNA molecule by reverse transcription, wherein the cDNA synthesis primer comprises a tag, and synthesizing the first cDNA strand thereby incorporates the tag into the cDNA to provide a plurality of tagged cDNA samples, wherein the cDNA in each tagged cDNA sample is complementary to RNA from a single cell, and wherein the tag comprises a unique molecular identifier (UMI) sequence and/or a barcode.

In some embodiments, the tag comprises a barcode without the UMI. In some embodiments, the tag comprises a UMI without the barcode.

Where desired, a given single cell workflow may include a pooling step where a cDNA product composition, e.g., made up of synthesized first strand cDNAs or synthesized double stranded cDNAs, is combined or pooled with the cDNA product compositions obtained from one or more additional cells. Accordingly, in some embodiments, such as when preparing a cDNA library for scRNA-seq, the method further comprises pooling the tagged cDNA samples; optionally amplifying the pooled cDNA samples to generate a cDNA library comprising double-stranded cDNA.

In some embodiments, addition of UMI and/or barcode can be performed by segregating individual cells into droplets. In some embodiments, the droplets are segregated from each other in an emulsion. In some embodiments, the droplets are formed and/or manipulated using a droplet actuator. In particular embodiments, one or more droplets comprise a different set of barcode-containing first strand synthesis primers. In some embodiments, each droplet comprises a multitude of first strand synthesis primers, each of these primers have identical sequence including identical barcodes and the barcodes from one droplet differ from another droplet, while the remaining portion of the first strand synthesis primer remains the same between the droplets. Thus, in these embodiments, the barcodes act as identifier for the droplets as well as well as the single cell encompassed by the droplet. In particular embodiments, one or more droplets comprise a different set of UMI-containing first strand synthesis primers. Thus, each individual cell that is lysed in each droplet will be identifiable by the barcodes in each droplet (DOI: https://doi.org/10.1016/j.cell.2015.04.044).

Examples of single-cell barcoding and sequencing techniques using droplet microfluidics systems for single-cell RNA-sequencing library preparation include, but are not restricted to, Drop-seq, inDrop, 10× Genomics automated single cell workflow—Chromium Single Cell 3′, to encapsulate cells in nano-liter microreactor droplets instead of reaction wells. Droplet-based single-cell RNA-seq methods are widely used high-throughput versions of single-cell RNA-sequencing, and widely known in the field of single-cell RNA-seq. These methods utilize water-in-oil droplets to compartmentalize single cells or the nucleus of single cells, and cDNA synthesis primers (poly(T), such as poly(dT)VN) with also contain sequenceable droplet-specific barcodes and nested PCR handles immobilized on a bead particles or soft hydrogel. In addition to the droplet- or cell-specific barcode, UMI sequences can be incorporated in the cDNA primers to count original mRNA transcripts during data analysis. Using microfluidics, droplets containing a single cell or a single-cell nucleus together with reverse transcriptase and RT buffer are fused with the droplets containing immobilized cDNA synthesis primers. mRNA priming and reverse transcription is then carried out in the fused droplets generating first strand cDNA uniquely barcoded for each droplet or cell. In one specific example, inDrops encapsulates cells by using hydrogel beads bearing poly(T) primers with defined barcodes, after which the photo-releasable primers are detached from the beads to improve molecule-capture efficiency and initiate in-drop RT reactions. After reverse transcription, the drops are broken and pooled, the cDNA is amplified by PCR, and 3′-end sequencing libraries are produced and amplified, e.g. by tagmentation using Tn5. The cell barcode sequences and UMIs are finally utilized to identify sequencing reads from the individual cells during downstream computational analysis.

With “droplet-based single-cell RNA-sequencing” we mean to include all methods in which the reverse transcription of the single-cell RNA is performed in a nanolitre droplets in oil emulsion, including the specific methods mention in the previous section.

Similarly, addition of UMI and/or barcode can be performed by segregating individual cells with beads that bear a UMI and/or barcode-tagged primer for first strand synthesis. Beads can be segregated into droplets in an emulsion. Beads can be segregated and manipulated using a droplet actuator (https://doi.org/10.1016/j.molcel2018.10.020).

In an embodiment, the agent is present in the first cDNA strand synthesis reaction at a concentration of 0.1-4000 μg/mL.

In an embodiment, the agent is present in the first cDNA strand synthesis reaction at a concentration of 0.1-4000 μg/mL, 500-4000 μg/mL, 1000-4000 μg/mL, 1000-3000 μg/mL, 100-3500 μg/mL, 0.1-2500 μg/mL, such as 10-1800 μg/mL, 50-2000, or 100-2000 μg/mL, such as 100-1000 μg/mL, or 100-750 μg/mL, or 100-500 μg/mL, such as 100-200 μg/mL. It will be appreciated that the agent may be present in the lysis buffer at a higher concentration, but following the direct addition of reagents for performing reverse transcription and first strand synthesis, the concentration of the agent decreases.

Polyvinylsulfonic acid (PVSA) is a organosulfur compound with the chemical formula (C₂H₃NaO₃S)n, and is a sulfonated polymer which contains repeated vinyl subunits.

As shown in the accompany Examples, when the exemplary agent was PVSA, the inventors prepared a lysis buffer (0.1% Triton X-100) containing 0-600 μg/mL of PVSA (resulting in 0-270 μg/mL in the following first strand synthesis reaction). The inventors identified that single-cell RNA-seq libraries constructed using PVSA were of similar size distribution and yield as standard Smart-seq2 (SS2) utilizing recombinant RNAse inhibitor (TaKaRa), with an identified optimal range of 30-120 μg/mL PVSA (FIG. 1b-c). At lower and higher PVSA concentrations the cDNA yield declined due to RNA degradation and reaction inhibition, respectively (FIG. 1b-c).

Vinylsulfonic acid (VSA) is a organosulfur compound with the chemical formula CH₂═CHSO₃H, and is a sulfonated monomer. Polymerization of VSA gives polyvinylsulfonic acid.

When the exemplary agent was VSA, the inventors prepared a 1× lysis buffer (0.1% Triton X-100) containing 100-3000 μg/mL of VSA (resulting in 45-1350 μg/mL in the following first strand synthesis reaction). Accordingly, in an embodiment when the agent is VSA, it is present in the first cDNA strand synthesis reaction at a concentration of 45-1350 μg/mL.

The inventors identified that RNA-seq libraries constructed using VSA were of similar size distribution and yield as standard Smart-seq2 utilizing recombinant RNAse inhibitor (TaKaRa), with an identified optimal range of 500-2000 μg/mL VSA.

Sodium alginate (NaC₆H₇O₆)n, NaAlg, is the sodium salt of alginic acid, and is a carboxylated polysaccharine derived from algae with a repeating disaccharide block. The structure of the repeating blocks are (1→4)-linked β-D-mannuronate (M) and α-L-guluronate (G) residues.

When the exemplary agent was sodium alginate, the inventors prepared a 1× lysis buffer (0.1% Triton X-100) containing 20-400 μg/mL of sodium alginate (resulting in 9-180 μg/mL in the following first strand synthesis reaction). Accordingly, in an embodiment when the agent is sodium alginate, it is present in the first cDNA strand synthesis reaction at a concentration of 9-180 μg/mL.

The inventors identified that RNA-seq libraries constructed using sodium alginate were of similar size distribution and yield as standard Smart-seq2 utilizing recombinant RNAse inhibitor (TaKaRa), with an identified optimal range of 200-400 μg/mL sodium alginate.

Heparin is a member of the glycosaminoglycan family of carbohydrates and consists of a variably sulfated repeating disaccharide unit. The repeating unit consists of is glucosamine and uronic acid.

When the exemplary agent was heparin, the inventors prepared a 1× lysis buffer (0.1% Triton X-100) containing 0.4-40 μg/mL of heparin (resulting in 0.18-18 μg/mL in the following first strand synthesis reaction). Accordingly, in an embodiment when the agent is heparin, it is present in the first cDNA strand synthesis reaction at a concentration of 0.18-18 μg/mL.

The inventors identified that RNA-seq libraries constructed using heparin were of similar size distribution and yield as standard Smart-seq2 utilizing recombinant RNAse inhibitor (TaKaRa), with an identified optimal range of 2-10 μg/mL heparin.

Dextran sulfate is a sulfated polymer consisting of (1→6)-α-linked anhydroglucose molecules. It has a molecular weight of greater than 500,000 Daltons.

When the exemplary agent was dextran sulfate, the inventors prepared a 1× lysis buffer (0.1% Triton X-100) containing 0.4-10 μg/mL of dextran sulfate (resulting in 0.18-4.5 μg/mL in the following first strand synthesis reaction). Accordingly, in an embodiment when the agent is dextran sulfate, it is present in the first cDNA strand synthesis reaction at a concentration of 0.18-4.5 μg/mL.

The inventors identified that RNA-seq libraries constructed using dextran sulfate were of similar size distribution and yield as standard Smart-seq2 utilizing recombinant RNAse inhibitor (TaKaRa), with an identified optimal range of 1-2.5 μg/mL dextran sulfate.

Fucoidan is a sulfated polysaccharine found in algae consisting predominantly fucose sugar molecules. It has a molecular weight ranging from approximately 50-1000 kiloDaltons.

When the exemplary agent was fucoidan, the inventors prepared a 1× lysis buffer (0.1% Triton X-100) containing 1-40 μg/mL of fucoidan (resulting in 0.45-18 μg/mL in the following first strand synthesis reaction). Accordingly, in an embodiment when the agent is fucoidan, it is present in the first cDNA strand synthesis reaction at a concentration of 0.45-18 μg/mL.

The inventors identified that RNA-seq libraries constructed using fucoidan were of similar size distribution and yield as standard Smart-seq2 utilizing recombinant RNAse inhibitor (TaKaRa), with an identified optimal range of 5-20 μg/mL fucoidan.

MES (C₆H₁₃NO₄S) or 2-(N-morpholino)ethanesulfonic acid is a organic compound consisting a morpholine ring with an ethane sulfonic acid group attached to the nitrogen in the ring.

When the exemplary agent was MES, the inventors prepared a 1× lysis buffer (0.1% Triton X-100) containing 2000-16000 μg/mL of MES (resulting in 900-7200 μg/mL in the following first strand synthesis reaction). Accordingly, in an embodiment when the agent is fucoidan, it is present in the first cDNA strand synthesis reaction at a concentration of 900-7200 μg/mL.

The inventors identified that RNA-seq libraries constructed using MES were of similar size distribution and yield as standard Smart-seq2 utilizing recombinant RNAse inhibitor (TaKaRa), with an identified optimal range of 4000-12000 μg/mL MES.

It will be appreciated that the particular concentration of the agent that is used (i.e. the concentration of the agent that is effective to inhibit the one or more RNase whilst not substantially interfering with the biological reactions necessary for the generation of a cDNA sequencing library) will be dependent on the identity of the agent used. The skilled person may readily determine what this concentration would be for any given agent. For example, different concentrations of agent could be tested for their effect on cDNA yield and/or quality using the methods described herein (such as by running a portion on an agarose or acrylamide gel or by using an instrument such as the Agilent Bioanalyzer) and the optimum concentration selected. Testing a range of concentrations may include testing 1, 4, 10, 40, 100, 400, 1000 μg/mL of agent in a aqueous solution, such as in a lysis buffer.

In a preferred embodiment, the method further comprises second cDNA strand synthesis from the first cDNA strand.

The second strand synthesis produces a second strand DNA complementary to the first strand cDNA, thus generating double stranded DNA.

The second cDNA strand is synthesized by a DNA or RNA-dependent DNA polymerase (including using the RT-synthesized DNA-strand as a template. Second-strand synthesis also requires a primer that is annealed to the first strand.

It will be appreciated that if a cDNA library is being prepared from, for example, 1 μg of RNA (such as from 30,000 cells), second strand synthesis may not be necessary.

However, if a cDNA library is being prepared from a single cell, second strand synthesis and further amplification will generally be necessary.

In a particular embodiment, second strand synthesis comprises RNA nicking and displacement; a primer that is complementary to an adapter pre-ligated to the 5′-end of the RNA template; and/or a template switching oligonucleotide (TSO) primer.

In an embodiment, second strand synthesis comprises RNA nicking and displacement. This procedure relies upon using a mix of DNA polymerase (such as E. coli DNA polymerase I), RNase (such as E. coli RNase H), and a DNA ligase (such as T4 DNA ligase). Like other polymerases, E. coli DNA polymerase I requires a primer-template duplex with a 3′-OH to initiate synthesis. Here, an RNase (such as RNase H) may be used to nick the original RNA template. The resulting RNA fragments can then function as primers to initiate synthesis off of the first-strand cDNA. During synthesis E. coli DNA polymerase I uses its 5′ to 3′ exonuclease activity to degrade on-coming RNA. Finally T4 DNA ligase repairs nicks that are left from the initial priming. This reaction has been well characterized and optimized, and is highly efficient. The primary drawback to this method is that the region corresponding to the 5′-terminal RNA is lost. This has little effect on gene expression studies but can be a problem for using RNA-seq to identify transcription start sites.

In an embodiment, second strand synthesis comprises an oligo ligated to the 5′-end of the RNA template. Several methods take advantage of pre-ligating an adapter to the 5′-end of the RNA template before the first-strand synthesis reaction, resulting in synthesis of a first-strand cDNA with a known sequence at the 3′-end. Oligos that are complementary to this adapter can then be used to prime second-strand synthesis. This technique allows one to recover intact 5′-ends of the template RNA, and is used in both the Small RNA kit (Illumina) and in the SOLiD RNA kits (Life Technologies).

In an embodiment, second strand synthesis comprises a template switching oligonucleotide (TSO) primer. Template switching is the ability of the certain reverse transcriptases (such as Moloney Murine Leukemia Virus (MMLV) reverse transcriptase) to introduce a few untemplated nucleotides, generally 2-5 cytosines, when it reaches the 5′-end of the RNA template, corresponding to the 3′-end of the newly synthesized cDNA strand. These extra nucleotides work as a docking site for a “Template Switching Oligonucleotide”, or TSO that, carries 2-5 (typically 3) complementary ribonucleotides, generally guanine ribonucleotides, i.e., rGrGrG at its 3′-end. The reverse transcriptase is then able to “switch template” (from mRNA to the DNA of the TSO) and synthesize a complementary DNA strand using an amplification primer and the TSO as template. Thus, template switching makes possible the introduction of an arbitrary sequence at the end of the transcript and, along with the known sequence located at the 5′-end of the first strand cDNA synthesis primer, allows the efficient amplification of all the transcripts in a cell in a subsequent amplification step, such as by PCR (Zhu Y Y, Machleder E M, et al. (2001) Biotechniques, 30(4):892-897.).

The template switch oligonucleotide may include one or more nucleotides (nts) (or analogues thereof) that are modified or otherwise non-naturally occurring. For example, the template switch oligonucleotide may include one or more nucleotide analogues (e.g., LNA, FANA, 2′-O-Me RNA, 2′-fluoro RNA, or the like), linkage modifications (e.g., phosphorothioates, 3′-3′ and 5′-5′ reversed linkages), 5′ and/or 3′ end modifications (e.g., 5′ and/or 3′ amino, biotin, DIG, phosphate, thiol, dyes, quenchers, etc.), one or more fluorescently labelled nts, or any other feature that provides a desired functionality to the template switch oligonucleotide. Any desired nucleotide analogues, linkage modifications and/or end modifications may be included in any of the nucleic acid reagents used when practicing the methods of the present disclosure.

The template switch oligonucleotide may include a 3′ hybridization domain and a 5′ amplification primer site. The 3′ hybridization domain may vary in length, and in some instances ranges from 2 to 10 nts in length, such as from 3 to 7 nts in length. The sequence of the 3′ hybridization domain, i.e., template switch domain, may be any convenient sequence, e.g., an arbitrary sequence, a heteropolymeric sequence (e.g., a hetero-trinucleotide) or homopolymeric sequence (e.g., a homo-trinucleotide, such as G-G-G), or the like. Examples of 3′ hybridization domains and template switch oligonucleotides are described in the Examples and further described in DOI: 10.2144/01304pf02, 10.1186/1471-2164-14-665, 10.1186/1471-2164-11-413, 10.1038/nprot.2014.006, the disclosures of which are herein incorporated by reference.

In an embodiment, the template switch oligo comprises a reverse amplification primer site, another primer site, such as a partial TN5 motif primers site, a novel identification tag, UMI and three rGs, and hybridizes to the non-templated nucleotides at the 3′ end of the cDNA strand. RT continues the polymerization using the TSO as a new template to get an extended cDNA strand that has a respective primer site at both ends (the other primer site having been provided by first strand cDNA synthesis primer). In some embodiments, usage of additional free ribonucleotides, dCTPs or PEG enable increased efficiency of the template switching reaction in terms of genes captured

Reverse transcriptases capable of template-switching that find use in practicing the methods include, but are not limited to, retroviral reverse transcriptase, retrotransposon reverse transcriptase, retroplasmid reverse transcriptases, retron reverse transcriptases, bacterial reverse transcriptases, group II intron-derived reverse transcriptase, and mutants, variants, derivatives, or functional fragments thereof, e.g., RNase FI minus or RNase FI reduced enzymes (e.g. Superscript RT or Maxima FI minus RT, Maxima H Minus RT (Thermo Fisher)). For example, the reverse transcriptase may be a Moloney Murine Leukemia Virus (MMLV) reverse transcriptase or a Bombyx mori reverse transcriptase (e.g., Bombyx mori R2 non-LTR element reverse transcriptase). Polymerases capable of template switching that find use in practicing the subject methods are commercially available and include SMARTScribe™ reverse transcriptase available from Takara Bio USA, Inc. (Mountain View, CA). In certain aspects, a mix of two or more different polymerases is added to the reaction mixture, e.g., for improved processivity, proof-reading, and/or the like.

The second strand synthesis primer may comprise a UMI and/or barcode as described above. The inclusion of a barcode at this stage in a scRNA-seq method allows early pooling of cells and cost-effective multiplex processing.

In a particular embodiment, the cDNA is amplified, optionally via in vitro transcription or by PCR using a first forward amplification primer and a first reverse amplification primer.

In an embodiment, the method comprises amplifying the tagged cDNA strand, or tagged cDNA fragments, optionally via in vitro transcription or by PCR using a first forward amplification primer and a first reverse amplification primer.

By “amplification” or “amplifying” we include the meaning of a process by which extra or multiple copies of a particular polynucleotide are formed. Amplification includes methods such as PCR, in vitro transcription (IVT), ligation amplification (or ligase chain reaction, LCR) and amplification methods. These methods are known and widely practiced in the art (see, for example, “PCR protocols: a guide to method and applications” Academic Press, Incorporated (1990) (for PCR); and Wu et al. (1989) Genomics 4:560-569 (for LCR).

In general, the PCR procedure describes a method of gene amplification which is comprised of (i) sequence-specific hybridization of primers to specific genes within a DNA sample (or library), (ii) subsequent amplification involving multiple rounds of annealing, elongation, and denaturation using a DNA polymerase, and (iii) screening the PCR products for a band of the correct size. The primers used are oligonucleotides of sufficient length and appropriate sequence to provide initiation of polymerization, i.e. each primer is specifically designed to be complementary to each nucleic acid strand to be amplified. Amplification conditions that may be employed include the addition of the one or more primers (e.g., as described above) and dNTPs. The conditions may include combining a thermostable polymerase (e.g., a Taq, Pfu, Tfl, Tth, Tli, and/or other thermostable polymerase) (if applicable, in addition to the template switching polymerase) into the reaction mixture. As shown in the accompanying Examples, amplification, e.g., PCR amplification, resulted in the production of a product double stranded cDNA.

The amplification step is preferably a PCR-based amplification using a polymerase, such as a Taq polymerase or a Phu polymerase or other DNA polymerases. Non-limiting examples of polymerases that could be used in the PCR-based amplification include Phusion High Fidelity DNA polymerase, Platinum SuperFi DNA polymerase, Q5 High Fidelity DNA polymerase, KAPA HiFi HotStart DNA polymerase, and TERRA™ PCR Direct polymerase.

Kits for assessing the quantity of cDNA are available and include Quant-iT PicoGreen dsDNA Assay Kit (Thermo Scientific) and Quibit 1× dsDNA kit (Thermo Scientific).

Reagents and hardware for conducting amplification reaction are commercially available. Primers useful to amplify sequences from a particular gene region are preferably complementary to, and hybridize specifically to sequences in the target region or in its flanking regions.

In order to amplify the cDNA by PCR or IVT, adaptor sequences (PCR) or RNA polymerase promoter sequences (T7 promoter) (IVT) are introduced during reverse transcription and/or second-strand synthesis. Amplification can be performed after the generation of the first cDNA strand, or in the same reaction mix and/or simultaneous as the reverse transcription reaction and, optionally template switching reaction. In an embodiment, the first strand synthesis primer (e.g. oligo-dT) and second strand synthesis (such as through the use of a TSO) each introduce either a forward or reverse amplification primer site, thus introducing said primer sites into the cDNA strands ready for subsequent amplification.

After amplification, the cDNA may be purified, for example, by using AMpure XP beads (Beckman Coulter) or PEG beads, gel purification or purification using a column. Purification can be performed for individual cells/samples as shown in the accompanying Examples, or it can be performed after pooling samples, if sample barcodes have already been inserted in a previous step.

In an embodiment, after amplification, such as by PCR, double-stranded cDNA is used for the generation of sequencing-ready libraries by using, for example, the Nextera XT DNA Library Preparation kit (Illumina). The Nextera XT kit relies on a hyperactive variant of a Tn5 transposase that carries out the fragmentation of double-stranded DNA and ligates synthetic oligonucleotides (“tags”) at both ends in a 5-minute reaction (Adey et al., 2010). Since the DNA is simultaneously tagged and fragmented, the reaction has been named “tagmentation”, discussed in detail below. A second PCR is then needed to append barcode adaptors for multiplexing.

In a particular embodiment, step (iii) comprises introducing one or more sequencing platform adapter sequences to the cDNA.

Sequencing platform adapter sequences, such as those discussed herein, can be added by any known method, such as, using Y-adapter PCR. Once the RNA is converted to double stranded cDNA the ends are blunted and adenosine overhangs are added. The adapter sequences can be added using a technique commonly referred to as Y-adapter PCR.

Alternatively, specific sequences can be added to each end of the insert during the first- and second-strand synthesis steps. In this case the first strand synthesis primer (e.g. oligo-dT primer) can contain an overhanging sequence that does not anneal to the RNA template but contains at least a portion of a sequencing platform adapter sequence. In a similar manner the forward amplification primer can contain over-hanging sequences. Two kits (SMARTer and ScriptSeq) use this approach.

Alternatively, adapters can be introduced via ligation. This approach is used in the Illumina TruSeq Small RNA kit, the NEBNext Small RNA prep kit, and in the SOLiD RNA kits from Life Technologies. These kits use ligation procedures that allow two different adapters to be ligated onto each end of the target RNA. These adapters are then used to prime the first- and second-strand synthesis reactions resulting in cDNAs terminated by the appropriate adapter sequences (RNA-seqlopedia).

Most current sequencing platforms are capable of providing only relatively short sequence reads (˜40-400 bp depending upon the platform). Therefore, most RNA-seq protocols incorporate a fragmentation step to improve sequence coverage over the transcriptome. However, protocols differ as to when the fragmentation is performed. Currently most RNA-seq cDNA libraries are constructed using RNA that has been fragmented as the initial template. However, there are situations where it is preferable to construct cDNA libraries using intact (i.e. unfragmented) RNA. Examples where this would be the case include using oligo-dT to prime first strand synthesis (as shown in the accompanying Examples), or where the goal is to sequence full-length RNA transcripts. In these situations it is necessary to fragment the double stranded cDNA before proceeding to the next step in the preparation of sequencing libraries.

Accordingly, in an embodiment, fragmentation is performed on cDNA.

In an embodiment, step (iii) comprises fragmentation of the cDNA.

In an alternative embodiment, fragmentation is performed on RNA. In an alternative embodiment, fragmentation is performed prior to step (ii) of the method.

By “fragmentation” we include the meaning of any protocol in which nucleic acid molecules are disrupted into shorter fragments. Methods used to fragment RNA include but are not limited to: moving an RNA sample one or more times through a micropipette tip or fine-gauge needle, nebulizing the sample, sonicating the sample (e.g., using a focused-ultrasonicator by Covaris, Inc. (Woburn, MA)), bead-mediated shearing, enzymatic shearing (e.g., using one or more RNA-shearing enzymes, or by enzymatic digestions, e.g., with restriction enzymes or other appropriate endonucleases), chemical based fragmentation, e.g., using divalent cations, fragmentation buffer (which may be used in combination with heat) or any other suitable approach for shearing/fragmenting RNA to generate a shorter template RNA. The RNA fragments generated by fragmentation of a starting RNA sample may have a length of from 10 to 20 nts, from 20 to 30 nts, from 30 to 40 nts, from 40 to 50 nts, from 50 to 60 nts, from 60 to 70 nts, from 70 to 80 nts, from 80 to 90 nts, from 90 to 100 nts, from 100 to 150 nts, from 150 to 200 nts, from 200 to 250 nts in length, or from 200 to 1000 nts or even from 1000 to 10,000 nts in length, for example, as appropriate for the sequencing platform chosen. Generally, the goal is to produce a population of RNA fragments that are on average about 200 bp.

In an embodiment, step (iii) comprises introducing one or more sequencing platform adapter sequences to cDNA fragments produced by fragmentation of the cDNA.

In an embodiment, the method also comprises fragmenting the resultant amplified cDNA molecules, e.g., using a fragmenting protocol as described above, followed by tagging the resultant fragments, e.g., for NGS. In some instances fragmenting and tagging the extended cDNA strand or an amplified version thereof is accomplished in a tagmentation process using a transposase and at least one tagging adapter to form tagged cDNA fragments (see Smart-seq2 (Picelli, S. et al. Nat. Methods 10, 1096-1098 (2013)) and Smart-seq3 (Hagemann-Jensen, M. et al., 2020 and WO2020136438A1).

Kits are available for tagmentation, for example, (“NEXTERA™”).

By “tagmentation” or “tagmenting” we include the meaning of modification of DNA by a transposome complex comprising transposase enzyme complexed with adaptors comprising transposon end domain. Tagmentation results in the simultaneous fragmentation of the DNA and ligation of the adaptors to the 5′ ends of both strands of duplex fragments. Following a purification step to remove the transposase enzyme, additional sequences can be added to the ends of the adapted fragments, for example by PCR, ligation, or any other suitable methodology known to those of skill in the art.

A “transposase” means an enzyme that is capable of forming a functional complex with a transposon end domain containing composition (e.g., transposons, transposon ends, transposon end compositions) and catalyzing insertion or transposition of the transposon end-containing composition into the double-stranded target DNA with which it is incubated in an in vitro transposition reaction.

The method of the invention can use any transposase that can accept a transposase end sequence and fragment a target nucleic acid, attaching a transferred end, but not a non-transferred end. Transposases that can be used with the methods of the present disclosure include, but are not limited to, Tn5 transposases, Tn7 transposases, and Mu transposases. The transposase may be a wild-type transposase. In other aspects, the transposase includes one or more modifications (e.g., amino acid substitutions) to improve a property of the transposase, e.g., enhance the activity of the transposase. For example, hyperactive mutants of the Tn5 transposase having substitution mutations in the Tn5 protein (e.g., E54K, M56A and L372P) have been developed and are described in, e.g., Picelli et al. (2013) Genome Research 24:2033-2040.

A “transposome” is comprised of at least a transposase enzyme and a transposase recognition site. Transposomes that may be employed in methods of the present disclosure include a transposase and a transposon nucleic acid that may include a transposon end domain among other domains. Any domains are defined functionally and so may be one in the same sequence or may be different sequences, as desired. The domains may also overlap.

The term “transposon end domain” means a double-stranded DNA that includes the nucleotide sequences (the “transposon end sequences”) that are necessary to form the complex with the transposase or integrase enzyme that is functional in an in vitro transposition reaction. A transposon end domain forms a complex with a transposase or integrase that recognizes and binds to the transposon end domain, and which complex is capable of inserting or transposing the transposon end domain into target DNA with which it is incubated in an in vitro transposition reaction.

In addition to the transposon end domain, the transposon nucleic acid may also include one or more additional domains, such as a post-tagmentation amplification primer binding site. In some instances, the post-tagmentation amplification primer binding site includes a sequencing platform adapter construct domain, e.g., as described above.

In an embodiment, step (iii) comprises an amplification step optionally via in vitro transcription (IVT) or by PCR using a second forward amplification primer and a second reverse amplification primer.

In an embodiment, sequencing adaptors are attached during a final amplification step.

In an embodiment, the first strand cDNA synthesis primer; and/or the first forward amplification primer; and/or first reverse amplification primer; and/or the TSO; and/or the second forward amplification primer; and/or second reverse amplification primer comprise: a unique molecular identifier (UMI); and/or multiple predefined nucleotides; and/or an amplification primer binding domain; and/or a barcode; and/or an adapter sequence.

It will be appreciated that to allow for subsequent amplification of the RNA by PCR or IVT, adaptors or T7 polymerase promoter sequences, respectively, are included in the first strand synthesis primer (such as in the oligo dT primer) and/or in the template switch primer and/or in the second strand synthesis primer.

Any nucleic acids that find use in practicing the methods of the present disclosure (e.g., the first strand cDNA primer, the template switch oligonucleotide, a second strand synthesis primer, one or more primers for amplifying the double stranded product nucleic acid, and/or the like) may include any useful nucleotide analogues and/or modifications, including any of the nucleotide analogues and/or modifications known in the art.

In a particular embodiment, the RNA sample comprises poly(A) containing RNA molecules, such as messenger RNA (mRNA) molecules, and the method comprises producing the cDNA sequencing library from mRNA.

It will be appreciated that cDNA is produced from fully transcribed mRNA found in a cell and therefore contains only the expressed genes of a single cell or when pooled together the expressed genes from a plurality of single cells.

Primer and adapter-dimer contamination in sequencing libraries can lead to serious problems like barcode switching (also called barcode hopping). Thus, these short molecules should be removed from the libraries as soon as traces of them become visible on the Bioanalyzer or equivalent.

As shown in the accompanying Examples, the inventors surprisingly found that the inclusion of such an agent, such as PVSA, increased the PCR specificity, reducing primer-dimer formation. Thus it will be appreciated that the invention includes a method of increasing PCR specificity in a method of preparing a cDNA sequencing library, the method comprising the use of an agent selected from the group consisting of: a sulfonated and/or carboxylated polymer; a sulfonated and/or carboxylated monomer; and a functionalised polysaccharide. Thus it will be appreciated that the invention includes a method of reducing primer dimer formation in a method of preparing a cDNA sequencing library, the method comprising the use of an agent selected from the group consisting of: a sulfonated and/or carboxylated polymer; a sulfonated and/or carboxylated monomer; and a functionalised polysaccharide.

By “PCR specificity” we include the meaning of the ability of a polymerase to preferentially amplify “on-target” sequences wherein primers base-pair at a higher complementarity to the preferred target DNA regions, resulting in “specific” PCR products, rather than “off-target” sequences where primers may still base-pair even though there will be a high level of mismatched bases to the less desired target DNA regions or oligos resulting in secondary, “non-specific” PCR products.

By “primer dimer” we include the meaning of a by-product of PCR, consisting of primer molecules that hybridize with each other because of strings of complementary bases in the primers. We also include the meaning of adapter dimers which are a pair of ligated adapters with no insert sequence. These adapter dimers can still be sequenced because they contain all of the relevant parts of the sequencing template, but will produce no useful sequence.

It will be appreciated that PCR specificity and primer-dimer formation can be determined by observing the production from the amplification on a gel. For single gene PCR, the size of the product is known, and any product outside of that size is a non-specific product. For cDNA production, amplified cDNA is a high molecular weight smear between 500 and 50,000 bp, and most primer-dimer products would be below 100 bp.

By “increasing PCR specificity” we include the meaning that PCR specificity during the generation of a cDNA sequencing library in the presence of the agent of the invention is increased, relative to the PCR specificity during the generation of a cDNA sequencing library in the absence of the agent of the invention.

By “reducing primer dimer formation” we include the meaning that primer dimer formation during the generation of a cDNA sequencing library in the presence of the agent of the invention is reduced, relative to the primer dimer formation during the generation of a cDNA sequencing library in the absence of the agent of the invention.

In an embodiment, the method is for preparing a single cell RNA sequencing (scRNAseq) library.

In an aspect, the invention provides a cDNA library obtained or obtainable by the method according to any embodiment disclosed herein.

In an aspect, the invention provides use of a cDNA library produced according to any embodiment disclosed herein for RNA sequencing (RNA-seq), such as single cell RNA-seq.

In certain embodiments, the subject methods may be used to generate a cDNA sequencing library for downstream sequencing on a sequencing platform of interest. The methods of the invention are not limited to any particular sequencing method. Sequencing of individual molecules or clonal populations can be carried out using known methods.

Full-length sequencing can be carried out, or 5′ or 3′ transcript ends can be selected for sequencing using specific amplification primers.

Sequencing can be paired-end or single end sequencing. Paired-end sequencing involves the sequencing of both ends of each cDNA fragment rather than sequencing only one end. Most current techniques are only capable of producing accurate sequence reads of 50-300 bases which is often less than the length of the insert. In order to increase the sequence coverage of inserts most platforms allow inserts to be sequenced from both ends. This technique (known as paired-end sequencing) can be used to increase the mapping accuracy and provides information that is useful for isoform detection. In order to use paired-end sequencing the adapters must contain a sequencing priming site that is situated on the opposite side of the insert

In an aspect, the invention provides use of an agent selected from the group consisting of: a sulfonated and/or carboxylated polymer; a sulfonated and/or carboxylated monomer; and a functionalised polysaccharide in a method of producing a cDNA sequencing library or in a method of in situ RNA-sequencing.

In situ RNA sequencing is known in the art and is described, for example in Ståhl et al. (Science 2016, Vol 353, Issue 6294, pp. 78-82).

In an embodiment, the invention provides use of an agent selected from the group consisting of: a sulfonated and/or carboxylated polymer; a sulfonated and/or carboxylated monomer; and a functionalised polysaccharide for reducing primer dimer formation during the generation of a cDNA sequencing library.

In an aspect, the invention provides a method for performing RNA sequencing (RNA-seq), such as single cell RNA-seq (scRNA-seq), the method comprising the steps of: preparing a cDNA library according to any embodiment disclosed herein; and sequencing the cDNA library.

In an aspect, the invention provides a lysis buffer comprising:
an agent selected from the group consisting of: a sulfonated and/or carboxylated polymer; a sulfonated and/or carboxylated monomer; and a functionalised polysaccharide; and
a detergent and/or chaotropic agent;
optionally comprising:
PEG;
BSA;
RNA spike-in;
dNTPs; and
first strand cDNA synthesis primer.

Detergents that may be used in the lysis buffer include, but are not limited to: Triton x100, SDS, NP-40/Igepal, Sarkosyl, Tween-20, sodium deoxycholate, and CHAPS. The detergent can be included in the lysis buffers at concentrations of 0.05-4%. Chaotropic agent that may be used in the lysis buffer include, but are not limited to guanidine salts, such as guanidinium thiocyanate or Guanidine hydrochloride (such as at 5M).

In an embodiment, the lysis buffer comprises at least one reverse transcription and/or amplification enhancer to promote enzymatic reaction rates of the reverse transcription and/or amplification reaction. Non-limiting, but illustrative, examples of such enhancers include betaine (such as at 1-2M), bovine serum albumin (BSA) (such as at 0.05-4%), glycerol, polyethylene glycol (PEG) (such as at 5-30%), glycogen, 1, 2-propanediol, dimethyl sulfoxide (DMSO), dimethylformamide (DMF), polyoxyethylene sorbitan monolaurate, such as polysorbate 20, polysorbate 40 and/or polysorbate 80, Beta mercaptoethanol (such as at 0.05-5 mM), T4 gene 32 protein and dithiothreitol (DTT) (such as at 1-10 mM).

Optionally, the lysis buffer may comprise a PEG having an average molecular weight selected within an interval of from 300 Da to 100,000 Da, preferably within an interval of from 1,000 to 25,000 Da, and more preferably within an interval of from 7,000 Da to 9,000 Da, such as 8000 Da. PEG, such as PEG 8000, acts a crowding agent causing a reduction in the effective reaction volume.

Optionally, the lysis buffer may comprise BSA.

By “RNA spike-in” we include the meaning of an RNA transcript of known sequence and quantity used to calibrate measurements in RNA hybridization assays, such as DNA microarray experiments, RT-qPCR, and RNA-Seq. RNA spike-in are available commercially, see for example, External RNA Controls Consortium (ERCC) spike-in mix 1 (Ambion).

Optionally, the lysis buffer may comprise dNTPs. In certain aspects, each of the four naturally-occurring dNTPs (dATP, dGTP, dCTP and dTTP) are added to the reaction mixture. For example, dATP, dGTP, dCTP and dTTP may be added to the reaction mixture such that the final concentration of each dNTP is from 0.01 to 100 mM, such as from 0.1 to 10 mM, including 0.5 to 5 mM (e.g., 1 mM). According to one embodiment, at least one type of nucleotide added to the reaction mixture is a non-naturally occurring nucleotide, e.g., a modified nucleotide having a binding or other moiety (e.g., a fluorescent moiety, biotin) attached thereto, a nucleotide analogue, or any other type of non-naturally occurring nucleotide that finds use in the subject methods or a downstream application of interest.

Optionally the lysis buffer comprises a first strand cDNA strand primer as described according to any embodiment disclosed herein.

In an embodiment, the sulfonated/carboxylated polymer is selected from the group consisting of: polyvinyl sulfonic acid (PVSA); the sulfonated/carboxylated monomer is selected from the group consisting of: vinyl sulfonic acid (VSA) and 2-(N-morpholino)ethanesulfonic acid (MES); the sulfonated/carboxylated polymer is selected from the group consisting of PVSA, and/or the functionalised polysaccharide is selected from the group consisting of: heparin, sodium alginate, dextran sulfate, and fucoidan.

According to one embodiment, the lysis buffer is present in a reaction tube (e.g., a 0.2 ml tube, a 0.6 ml tube, a 1.5 ml tube, or the like) or a well, or microfluidic chamber, or droplet, or other suitable container. In certain aspects, the lysis buffer is present in two or more (e.g., a plurality of) reaction tubes or wells (e.g., a plate, such as a 96-well plate, a multi-well plate, e.g., containing about 1000, 5000, or 10,000 or more wells). The tubes and/or plates may be made of any suitable material, e.g., polypropylene, or the like, PDMS, or aluminium. In certain aspects, the tubes and/or plates in which the lysis buffer is present provide for efficient heat transfer to the composition (e.g., when placed in a heat block, water bath, thermocycler, and/or the like), so that the temperature of the composition may be altered within a short period of time, e.g., as necessary for a particular enzymatic reaction to occur. According to certain embodiments, the lysis buffer is present in a thin-walled polypropylene tube, or a plate having thin-walled polypropylene wells or materials such as aluminium having high heat conductance. In some instances, the lysis buffer of the invention may be present in droplets. In certain embodiments it may be convenient for the first strand and/or second strand synthesis reaction to take place on a solid surface or a bead, in such case, the first strand cDNA primer and/or template switch oligonucleotide, or one or more other primers, may be attached to the solid support or bead by methods known in the art—such as biotin linkage or by covalent linkage—and reaction allowed to proceed on the support. Alternatively, the oligos may be synthesized directly on the solid support.

In an embodiment, wherein the lysis buffer is present at a 1× concentration, the agent is present at 0.1-8000 μg/mL.

By “1× concentration” we include the meaning of the working concentration of lysis buffer that is used to contact the cells. It will be appreciated that stock lysis buffer can be prepared in a more concentration form, such as 5× or 10× concentrated, but can be diluted to a 1× working concentration prior to use.

As shown in the accompany Examples, when the exemplary agent was PVSA, the inventors prepared a 1× lysis buffer (0.1% Triton X-100, 2.5 mM dNTPs, 2.5 mM Smart-seq2 oligo-dT primer) containing 0-600 μg/mL of PVSA (resulting in 0-270 μg/mL in the following first strand synthesis reaction). The inventors identified that single-cell RNA-seq libraries constructed using PVSA were of similar size distribution and yield as standard Smart-seq2 utilizing recombinant RNAse inhibitor (TaKaRa), with an identified optimal range of 30-120 μg/mL PVSA (FIG. 1b-c). At lower and higher PVSA concentrations the cDNA yield declined due to RNA degradation and reaction inhibition, respectively (FIG. 1b-c). The quality of the sequencing data obtained from PVSA libraries (e.g. number of genes detected; fraction reads mapping to exonic and intronic regions; frequency of base errors such as single base substitutions, insertions, and deletions; and read-coverage along the length of transcripts) were on par with standard Smart-seq2 utilizing recombinant RNAse inhibitor (TaKaRa) when PVSA was used in the optimal range (FIG. 1f-j, FIG. 2d-n, FIG. 4, FIG. 6).

The inventors prepared 1× lysis buffer (0.1% Triton X-100, 0.5 mM dNTPs, 1 μM Smart-seq3 Oligo-dT primer) containing 0-90 μg/mL of PVSA (resulting in 0-45 μg/mL in the following first strand synthesis reaction). They identified that single-cell RNA-seq libraries constructed using PVSA were of similar size distribution and yield as standard Smart-seq3 utilizing recombinant RNAse inhibitor (TaKaRa), with an identified optimal range of 0.3-15 μg/mL PVSA (FIG. 2o-p). The quality of the sequencing data obtained from PVSA libraries were on par with standard Smart-seq3 utilizing recombinant RNAse inhibitor (TaKaRa) when PVSA was used in the identified optimal range (FIG. 2s-v and FIG. 7).

The inventors prepared 1× lysis buffer (0.1% Triton X-100, 0.5 mM dNTPs, 0.125 μM Smart-seq3 Oligo-dT primer) containing 0-30 μg/mL of PVSA (resulting in 0-22.5 μg/mL in the following first strand synthesis reaction). They identified that single-cell RNA-seq libraries constructed using PVSA in the Smart-seq3xpress protocol yielded improved scRNA-seq libraries in terms of genes detected compared to standard Smart-seq3xpress utilizing recombinant RNAse inhibitor (TaKaRa), with an identified optimal range of 0.6-3 μg/mL PVSA (FIG. 13a-d). When the inventors incubated collected single cells from a human kidney cell line in Smart-seq3xpress lysis buffers containing 0-30 μg/mL of PVSA or recombinant RNAse inhibitor (TaKaRa) for up to 7 days in room temperature (25° C.) or up to 14 days in refrigerator (4° C.) and then prepared sequencing libraries from the cells, they found that libraries containing PVSA retained quality in terms of number of genes and UMIs detected per cell better than standard Smart-seq3xpress recombinant RNAse inhibitor (TaKaRa) or no inhibitor (FIG. 13e-h).

When the exemplary agent was VSA, the inventors prepared a 1× lysis buffer (0.1% Triton X-100, 2.5 mM dNTPs, 2.5 mM Smart-seq2 oligo-dT primer) containing 100-3000 μg/mL of VSA (resulting in 45-1350 μg/mL in the following first strand synthesis reaction). The inventors identified that RNA-seq libraries constructed using VSA were of similar size distribution and yield as standard Smart-seq2 utilizing recombinant RNAse inhibitor (TaKaRa), with an identified optimal range of 500-2000 μg/mL VSA (FIG. 15f). The quality of the sequencing data obtained from VSA libraries were on par with standard Smart-seq2 utilizing recombinant RNAse inhibitor (TaKaRa) when PVSA was used in the identified optimal range (FIG. 15j-1).

When the exemplary agent was sodium alginate, the inventors prepared a 1× lysis buffer (0.1% Triton X-100, 2.5 mM dNTPs, 2.5 mM Smart-seq2 oligo-dT primer) containing 20-400 μg/mL of sodium alginate (resulting in 9-180 μg/mL in the following first strand synthesis reaction). The inventors identified that RNA-seq libraries constructed using sodium alginate were of similar size distribution and yield as standard Smart-seq2 utilizing recombinant RNAse inhibitor (TaKaRa), with an identified optimal range of 200-400 μg/mL sodium alginate (FIG. 15e). The quality of the sequencing data obtained from sodium alginate libraries were on par with standard Smart-seq2 utilizing recombinant RNAse inhibitor (TaKaRa) when sodium alginate was used in the identified optimal range (FIG. 15j-1).

When the exemplary agent was heparin, the inventors prepared a 1× lysis buffer (0.1% Triton X-100, 2.5 mM dNTPs, 2.5 mM Smart-seq2 oligo-dT primer) containing 0.4-40 μg/mL of heparin (resulting in 0.18-18 μg/mL in the following first strand synthesis reaction). The inventors identified that RNA-seq libraries constructed using heparin were of similar size distribution and yield as standard Smart-seq2 utilizing recombinant RNAse inhibitor (TaKaRa), with an identified optimal range of 2-10 μg/mL heparin (FIG. 15d). The quality of the sequencing data obtained from heparin libraries were on par with standard Smart-seq2 utilizing recombinant RNAse inhibitor (TaKaRa) when heparin was used in the identified optimal range (FIG. 15j-1).

When the exemplary agent was dextran sulfate, the inventors prepared a 1× lysis buffer (0.1% Triton X-100, 2.5 mM dNTPs, 2.5 mM Smart-seq2 oligo-dT primer) containing 0.4-10 μg/mL of dextran sulfate (resulting in 0.18-4.5 μg/mL in the following first strand synthesis reaction). The inventors identified that RNA-seq libraries constructed using dextran sulfate were of similar size distribution and yield as standard Smart-seq2 utilizing recombinant RNAse inhibitor (TaKaRa), with an identified optimal range of 1-2.5 μg/mL dextran sulfate (FIG. 15g). The quality of the sequencing data obtained from dextran sulfate libraries were on par with standard Smart-seq2 utilizing recombinant RNAse inhibitor (TaKaRa) when dextran sulfate was used in the identified optimal range (FIG. 15j-1).

When the exemplary agent was fucoidan, the inventors prepared a 1× lysis buffer (0.1% Triton X-100, 2.5 mM dNTPs, 2.5 mM Smart-seq2 oligo-dT primer) containing 1-40 μg/mL of fucoidan (resulting in 0.45-18 μg/mL in the following first strand synthesis reaction). The inventors identified that RNA-seq libraries constructed using fucoidan were of similar size distribution and yield as standard Smart-seq2 utilizing recombinant RNAse inhibitor (TaKaRa), with an identified optimal range of 5-20 μg/mL fucoidan (FIG. 15h). The quality of the sequencing data obtained from fucoidan libraries were on par with standard Smart-seq2 utilizing recombinant RNAse inhibitor (TaKaRa) when fucoidan was used in the identified optimal range (FIG. 15j-1).

When the exemplary agent was MES, the inventors prepared a 1× lysis buffer (0.1% Triton X-100, 2.5 mM dNTPs, 2.5 mM Smart-seq2 oligo-dT primer) containing 2000-16000 μg/mL of MES (resulting in 900-7200 μg/mL in the following first strand synthesis reaction). The inventors identified that RNA-seq libraries constructed using MES were of similar size distribution and yield as standard Smart-seq2 utilizing recombinant RNAse inhibitor (TaKaRa), with an identified optimal range of 4000-12000 μg/mL MES (FIG. 15i). The quality of the sequencing data obtained from MES libraries were on par with standard Smart-seq2 utilizing recombinant RNAse inhibitor (TaKaRa) when MES was used in the identified optimal range (FIG. 15j-1).

In an embodiment, the method, use, and lysis buffer does not comprise a biological RNase inhibitor.

By “biological RNase inhibitor” we include the meaning of an RNase inhibitor derived from a biological source, such as from mammalian liver or placenta, such as a recombinant human placental protein. The biological RNase inhibitor may comprise a polypeptide.

Examples of biological RNase inhibitors include recombinant RNase inhibitors. By “recombinant RNase inhibitor” we include the meaning of an RNase encoded by recombinant DNA that has been cloned in an expression vector that supports expression of the gene and translation of messenger RNA. Non-limiting examples include E. coli containing a plasmid that carries the porcine RNase Inhibitor gene, cloned porcine liver RNase (RNaseOUT™); human placenta RNase (RNasein); SUPERase-In; recombinant RNase from rat lung ((Protector RNase Inhibitor (Roche)); E. coli cells with a cloned gene encoding a mammalian RNase Inhibitor gene (RiboLock RNase Inhibitor:); a murine RNase inhibitor (NEB); and an RNase inhibitor from human placenta (NEB). The amino acid sequences of currently used recombinant inhibitors have generally been modified in vitro as to maximize their ability to inhibit RNases.

As shown in the accompany Examples, the inventors used Nuclear Magnetic Resonance (NMR) spectroscopy to demonstrating that PVSA interacts extensively with RNase A protein (FIG. 18) while no or little not interaction was observed between PVSA and a 14-mer hairpin RNA (FIG. 19), indicating that PVSA inhibits RNA degradation primarily by interacting with RNase.

As discussed herein, the inhibitory agents can be bound to the surfaces (e.g., glass, plastic, or fiber material) of containers, surfaces, or other equipment (e.g., multi-well plates, etc.) in which biological media, including buffers, are stored or in which biological purifications, reactions and or assays are carried out during the process of preparing a cDNA sequencing library.

Accordingly, the invention also provides a solid support comprising the agent as defined in any of the embodiments herein.

Various solid supports are known in the art and include, for example, fibrous materials, tubes, plate (such as a multi-well plate), beads, columns, slides, sheets, chips, foams and sponges.

It will be appreciated that the solid support is capable of binding to the agent defined herein, or otherwise containing the agent defined herein. By “binding to the agent” we include the meaning that the agent is immobilised onto a solid support. Immobilisation may be via a covalent or non-covalent interaction.

Preparation of agent-coated supports can be achieved using an in situ polymerization method or by incubating material or surfaces with the inhibitory agent. Those of ordinary skill in the art will appreciate that other means for directly or indirectly (through a linker) coupling of agents of this invention to solid supports are available in the art and can be employed in the practice of this invention.

Where immobilisation is via a non-covalent interaction, the support may be coated with a moiety that binds non-covalently to the agent. Additionally or alternatively, the agent can be adsorbed to the support either through the porous nature of the support or through weak hydrophobic and/or polar interactions between the support and the agent. Any suitable system for non-covalent interactions may be used, including any of ELISA principles, hydrophobic-hydrophilic interactions, adsorption, absorption, and high binding polystyrene through radiation. Also, any suitable commercially available support that allows for non-covalent interactions can be used, such as those available from Corning®.

Where immobilisation is via a covalent interaction, the support may be coated with a pre-activated functional group to covalently immobilize the agent to the surface. Any suitable system for covalent interactions may be used, including ELISA principles, and pre-activated surfaces to facilitate covalent bonds. Also, any suitable commercially available support that allows for covalent interactions can be used, such as those available from Corning®.

By “containing” we include the meaning that the solid support has one or more pores within which the agents as defined herein can be retained. In most cases, biological samples (e.g., whole blood, saliva, urine tissue and cells and lysates thereof) need be stored and transported at low temperature before analysis. Tubes, bottles and refrigerators are normally utilized to collect and store samples. Compared to these methods, paper has the advantages of low cost, porous structure, portability and ease of use. Thus, improved paper-based sample storage and collection methods are required.

The inventors have shown that fibrous material pre-incubated with the agents defined herein allow storage and collection of RNA containing samples at room temperature and the eluted RNA is compatible with RNA sequencing. The fibrous material can be stored at room temperature. After storage, the RNA can be eluted and analysed for RNA quality, cDNA yield by methods known in the art and as described herein.

In an embodiment, the solid support is a fibrous material, such as paper. Fibrous materials may comprise cotton fibre, glass fibre, polymer, cellulose, or a combination thereof. It will be appreciated that the fibrous material is suitable for binding RNA. Chemical groups on paper surface (e.g., hydroxyl and carboxyl groups) can help immobilize chemical reagents on paper. For example, cellulose contains hydroxyl groups on its surface and has the properties of hydrophilicity, easy usability, high porosity, high mechanical strength. Cotton fiber, a natural material, also contains hydroxyl groups on its surface. Glass fiber is a kind of synthetic fiber and is formed of silica-based thin strands. Such fibrous material can be 100 μm-500 μm thick. Examples of fibrous material include filter paper such as Flinders Technology Associates (FTA) Cards®, Nobuto filter paper, Whatman® paper, and iBlot Filter Paper.

It will be appreciated that the fibrous material is suitable for receiving a liquid solution comprising the agent. In an embodiment, the fibrous material is pre-incubated with a liquid solution comprising an effective amount of the agent. Effective amounts include 10 μg agent/mL water to 1000 mg agent/mL water.

As shown in the accompanying Examples, the inventors soaked cotton paper sheets in aqueous solution containing 0-30 mg/mL PVSA and let the paper dry. The inventors then spotted Triton-X100-lysed cells on the papers sheets and incubated the sheets in room temperature (25° C.) for up to seven days. When the inventors prepared full-length RNA-seq cDNA libraries using bulk Smart-seq2, they found that the mRNA of lysed cells was protected from degradation in a PVSA-concentration-dependent manner, as demonstrated by cDNA traces (FIG. 20). Notably, the size distribution of cDNA libraries constructed from lysed cells stored for seven days on paper sheets soaked in 30 mg/mL had the shape of intact mRNA, comparable to cDNA libraries prepared from day 0 control samples (FIGS. 20c and e).

The soaking of a paper sheet may alternatively be in a solution of PVSA in a concentration of 3-300 mg/mL. The paper sheet may be dried before use of the paper.

The paper may be used as storage for RNA of a biological sample. The sample may be lysed before it is applied to the paper. The sample may comprise one or more cells, and may be stored for 0, 2, 3, 5, 7, or more days. The RNA may subsequently be used for generation of an RNA sequencing library.

In an embodiment the paper sheet is made of cotton fiber.

In an embodiment, the solid support is a multi-well plate. In an embodiment, the multi-well plate comprises a aqueous solution, such as a storage buffer or lysis buffer as described herein which comprises the agent.

By “multi-well plate” or “microtiter plate” we include the meaning of a flat plate with multiple “wells” used as small test tubes. The multiwell plate may have a plurality of microwells, nanowells or picowells. For example, Seq-Well provides a nanowell-based method that captures cells in 86,000 sub-nanoliter reactions. For example a 96-well plate, 384 well plate, or a plate with any number of wells such as 2000, 4000, 6000, or 10000 or more. The multi-well plate can be part of a chip and/or device. The present disclosure is not limited by the number of wells in the multi-well plate in various embodiments, the total number of wells on the plate is from 96 to 200,000, or from 5,000 to 10,000. The plate may comprise smaller chips, each of which includes 5,000 to 20,000 wells. For example, a square chip may include 125 by 125 nanowells, with a diameter of 0.1 mm. The wells (e.g., nanowells) in the multi-well plates may be fabricated in any convenient size, shape or volume. The well may be 100 mm to 1 mm in length, 100 μm to 1 mm in width, and 100 μm to 1 mm in depth. Each nanowell may have an aspect ratio (ratio of depth to width) of from 1 to 4. The transverse sectional area may be circular, elliptical, oval, conical, rectangular, triangular, polyhedral, or in any other shape. The transverse area at any given depth of the well may also vary in size and shape. The wells may have a volume of from 0.1 nl to 1 mL. The nanowell may have a volume of 1 mL or less, such as 500 nl or less. The volume may be 200 nl or less, such as 100 nl or less. In an embodiment, the volume of the nanowell is 100 nl. Where desired, the nanowell can be fabricated to increase the surface area to volume ratio, thereby facilitating heat transfer through the unit, which can reduce the ramp time of a thermal cycle. The cavity of each well (e.g., nanowell) may take a variety of configurations. For instance, the cavity within a well may be divided by linear or curved walls to form separate but adjacent compartments, or by circular walls to form inner and outer annular compartments. The wells can be designed such that a single well includes a single cell.

A microreactor droplet comprising lysis buffer according to any embodiment disclosed herein.

By “microreactor droplet” we include the meaning of pico- or nanoliter scale droplets emulsions, which are created in a microfluidic device.

Droplet-based systems such as inDrops, Drop-seq, 10× Genomics automated single cell workflow, and Chromium Single Cell 3′ encapsulate cells in nanoliter microreactor droplets. Both inDrops, Drop-seq utilize water-in-oil droplets to compartmentalize barcodes and single cells and then lyse the cell for RT and barcoding of the cDNA, thus performing cell isolation, lysis, and molecular processing all at once. inDrops encapsulates cells by using hydrogel beads bearing poly(T) primers with defined barcodes, after which the photo-releasable primers are detached from the beads to improve molecule-capture efficiency and initiate in-drop RT reactions. The barcoded cDNAs are then pooled for linear amplification (IVT) and 3′-end sequencing-library preparation. Unlike inDrops protocols, Drop-sew and 10× Genomics' workflow uses oligo dT beads with random barcodes utilized to identify sequencing reads from the individual cells. After cell lysis and RNA capture, the drops are broken and pooled, covalent binding is carried out through cDNA synthesis, the cDNA is amplified by PCR, and 3′-end sequencing libraries are produced.

Any suitable system for forming and manipulating droplets can be used (Drop-seq Macosko, E. Z. et al. Cell 161, 1202-1214 (2015)). Droplets may, for example, be aqueous or non-aqueous or may be mixtures or emulsions including aqueous and non-aqueous components. A droplet may include one or more beads.

The droplet may further comprise nucleic acids, such as DNA, genomic DNA, RNA, mRNA or analogs thereof. Other examples of droplet contents include any reagent described herein, such as for a nucleic acid amplification protocol.

Cells present in each droplet may be lysed in parallel by the lysis buffer of the invention that has been incorporated in the aqueous phase.

Agents of the invention may be used in micro-droplet-based systems for single-cell RNA-sequencing methods such as inDrops, Drop-seq, 10× Genomics automated single cell workflow, and Chromium Single Cell 3′. Such methods utilise water-in-oil droplets to compartmentalize barcodes and single cells and then lyse the cell for RT and barcoding of the cDNA, thus performing cell isolation, lysis, and molecular processing all at once. inDrops encapsulates cells by using hydrogel beads bearing poly(T) primers with defined barcodes, after which the photo-releasable primers are detached from the beads to improve molecule-capture efficiency and initiate in-drop RT reactions. The barcoded cDNAs are then pooled for amplification and 3′-end sequencing-library preparation. Unlike inDrops protocols, Drop-seq and 10× Genomics' workflow uses beads covered with oligonucleotide sequence containing oligo dT sequence (such as poly(dT)VN), a random barcode sequence utilized to identify sequencing reads from the individual cells, optionally a random UMI sequence, and a nested sequence used to prime an amplification reaction and/or to prime the following sequencing. After cell lysis and RNA capture, the drops are broken and pooled, covalent binding is carried out through cDNA synthesis, the cDNA is amplified by PCR, and 3′-end sequencing libraries are produced by tagmentation. Aqueous solution of agents of the invention may be used in such micro-droplet-based methods. Thus, PVSA may be used in any one of these methods to encapsulate cells in nanoliter microreactor droplets instead of in reaction wells. PVSA may be in the form of an aqueous solution. Preferably the concentration of PVSA in such a solution is 0.1-120 μg/mL, such as from 0.3 to 110 μg/mL, such as from 0.5 to 100 μg/mL, such as from 1 to 90 μg/mL, such as from 2 to 80 μg/mL, such as from 5 to 70 μg/mL, such as from 10 to 60 μg/mL, such as from 15 to 50 μg/mL, such as from 20 to 40 μg/mL, such as 30 μg/mL.

In an aspect, the invention provides a method for lysing one or more cells and releasing RNA molecules, wherein the method comprises contacting one or more cells with a lysis buffer according to any embodiment disclosed herein in order to provide a plurality of RNA molecules.

In a particular embodiment, the one or more cells are spatially separated into single cells prior to contact with a lysis buffer such that a plurality of individual RNA samples is provided, wherein each individual RNA sample comprises a plurality of RNA molecules from a single cell.

Aspects of the present disclosure also include kits. As used herein, the term “kit” refers to one or more suitably aliquoted compositions or reagents for use in the methods of the present disclosure. The components of the kits may be packaged either in aqueous or lyophilized form. The container means of the kits may include at least one vial, test tube, flask, bottle, syringe, or other container means, into which a component may be placed, and preferably, suitably aliquoted. Where there is more than one component in the kit, the kit also will generally contain a second, third, or other additional container into which the additional components may be separately placed. However, various combinations of components may be contained in a vial. The kits of the present disclosure also will typically include a means for containing the reagent containers in close confinement for commercial sale. Such containers may include injection or blow moulded plastic containers into which the desired vials are retained, for example.

The kit may also comprise any of a TSO, a reducing agent (e.g. DTT) and a buffer (such as any of those described herein).

In an embodiment, the kit comprises an agent selected from the group consisting of: a sulfonated and/or carboxylated polymer; a sulfonated and/or carboxylated monomer; and a functionalised polysaccharide as described according to any embodiment disclosed herein.

In a particular embodiment, the sulfonated/carboxylated polymer is selected from the group consisting of: polyvinyl sulfonic acid (PVSA); the sulfonated/carboxylated monomer is selected from the group consisting of: vinyl sulfonic acid (VSA) and 2-(N-morpholino)ethanesulfonic acid (MES); and/or the functionalised polysaccharide is selected from the group consisting of: heparin sodium alginate, dextran sulfate, and fucoidan.

In an embodiment, the kit comprises a first strand cDNA synthesis primer (such as an oligo-dT primer) according to any embodiment disclosed herein. In an embodiment, the kit comprises a plurality of first strand synthesis primers. The plurality of first strand synthesis primers differ from each other by UMI, such that each comprises a UMI that is unique and different from UMIs of other first strand cDNA synthesis primers.

In an embodiment, the kit comprises a mixture dATP, dGTP, dTTP and dCTP.

In an embodiment, the kit comprises a strand switch primer (such as a TSO) according to any embodiment disclosed herein. In an embodiment, the kit comprises a plurality of strand switch primers, such as a set of TSOs. In an embodiment, the kit comprises a set of second strand synthesis primers (e.g. TSOs) that differ from each other by UMI, such that each comprises a UMI that is unique and different from UMIs of other strand switch primers. For instance, a set of 65,536 unique strand switch primers with different UMIs can be obtained with a UMI length of 8 nucleotides.

In an embodiment, the kit comprises a reverse transcriptase. The reverse transcriptase is preferably selected among the previously described examples of reverse transcriptases.

The kit may include components for second strand synthesis such as a first forward amplification primer; and/or first reverse amplification primer; and/or the second forward amplification primer; and/or second reverse amplification primer.

In an embodiment, the kit comprises at least one reverse transcription and/or amplification enhancer. The at least one such enhancer is preferably selected among the previously described examples of enhancers.

The kits may further include one or more of a salt, a metal cofactor, one or more molecular crowding agents (e.g., PEG, or the like), one or more enzyme-stabilizing components (e.g., DTT), or any other desired kit component(s), such as solid supports, e.g., tubes, beads, microfluidic chips, etc.

The kit may include reagents for isolating a nucleic acid sample from a fixed cell, tissue or organ, e.g., formalin-fixed, paraffin-embedded (FFPE) tissue. Such kits may include one or more deparaffinization agents, one or more agents suitable to de-crosslink nucleic acids, and/or the like.

In embodiments there are provided kits for preparing cDNA sequencing libraries. Such kits may comprise a first container containing an aqueous solution of PVSA or another agent according to the invention capable of RNase inhibition; and a second container comprising a lysis buffer. The lysis buffer may further comprise dNTPs and/or primers for reverse transcription. It is appreciated that none of the containers comprise a recombinant RNase inhibitor. The agent may e.g. be PVSA in a concentration range of from 0.1 to 2000 μg/mL, such as from 0.1 to 1000 μg/mL, as from 0.1 to 500 μg/mL, such as from 0.1 to 200 μg/mL, such as from 0.1 to 180 μg/mL, such as from 0.1 to 150 μg/mL, such as from 0.1 to 120 μg/mL, such as from 0.1 to 100 μg/mL, such as from 0.1 to 90 μg/mL.

A kit for preparing a cDNA sequencing library comprising:
an agent selected from the group consisting of: a sulfonated and/or carboxylated polymer; a sulfonated and/or carboxylated monomer; and a functionalised polysaccharide; and
a first strand cDNA synthesis primer;
   and optionally
dNTPs;
a second strand synthesis primer.

A kit for preparing a cDNA sequencing library comprising:
an agent selected from the group consisting of: an Polyvinylsulfonic acid (PVSA), Vinylsulfonic acid (VSA), Alginate, heparin, Dextran sulfate, Fucoidan, 2-(N-morpholino)ethanesulfonic acid (MES);
   a detergent for cell lysis;
      and optionally
   a first strand cDNA synthesis primer;
   dNTPs;
   a second strand synthesis primer.

A kit for preparing a cDNA sequencing library comprising:
an Polyvinylsulfonic acid (PVSA) in aqueous, as RNase inhibitor.
a detergent for cell lysis;
and optionally:
a first strand cDNA synthesis primer;
dNTPs;
a second strand synthesis primer.

A kit for preparing a cDNA sequencing library comprising:
Vinylsulfonic acid (VSA) in aqueous, as RNase inhibitor.
a detergent for cell lysis;
and optionally:
a first strand cDNA synthesis primer;
dNTPs;
a second strand synthesis primer.

A kit for preparing a cDNA sequencing library comprising:
Sodium alginate in aqueous, as RNase inhibitor.
a detergent for cell lysis;
and optionally:
a first strand cDNA synthesis primer;
dNTPs;
a second strand synthesis primer.

A kit for preparing a cDNA sequencing library comprising:
Heparin in aqueous, as RNase inhibitor.
a detergent for cell lysis;
and optionally:
a first strand cDNA synthesis primer;
dNTPs;
a second strand synthesis primer.

A kit for preparing a cDNA sequencing library comprising:
Dextran sulfate in aqueous, as RNase inhibitor.
a detergent for cell lysis;
and optionally:
a first strand cDNA synthesis primer;
dNTPs;
a second strand synthesis primer.

A kit for preparing a cDNA sequencing library comprising:
Fucoidan in aqueous, as RNase inhibitor.
a detergent for cell lysis;
and optionally:
a first strand cDNA synthesis primer;
dNTPs;
a second strand synthesis primer.

A kit for preparing a cDNA sequencing library comprising:
2-(N-morpholino)ethanesulfonic acid (MES) in aqueous, as RNase inhibitor.
a detergent for cell lysis;
and optionally:
a first strand cDNA synthesis primer;
dNTPs;
a second strand synthesis primer.

In an embodiment, the agent is selected from the group consisting of: polyvinyl sulfonic acid (PVSA), vinyl sulfonic acid (VSA), heparin, sodium alginate, dextran sulfate, fucoidan, 2-(N-morpholino)ethanesulfonic acid (MES), sulfated cellulose, sulfated amylose, sulfated pectic acid, sulfated polyvinyl alcohol. In an embodiment the kit comprises a tube or similar container comprising a cell lysis buffer comprising an agent and a detergent. In an embodiment the agent is PVSA in the concentration range of from 0.1 to 120 μg/mL such as from 0.1 to 1000 μg/mL, such as from 0.1 to 500 μg/mL, such as from 0.1 to 200 μg/mL, such as from 0.1 to 180 μg/mL, such as from 0.1 to 150 μg/mL, such as from 0.1 to 120 μg/mL, such as from 0.1 to 100 μg/mL, such as from 0.1 to 90 μg/mL. In an embodiment the tube further contains dNTPs and oligo dT primers. Such kits are intended for RNA sequencing applications such as single-cell RNA sequencing applications.

In another embodiment the kit comprises a tube containing only PVSA and water. The concentration of PVSA may be in the range of from 0.1 to 2000 μg/mL, such as from 0.1 to 1000 μg/mL, such as from 0.1 to 500 μg/mL, such as from 0.1 to 200 μg/mL, such as from 0.1 to 180 μg/mL, such as from 0.1 to 150 μg/mL, such as from 0.1 to 120 μg/mL, such as from 0.1 to 100 μg/mL, such as from 0.1 to 90 μg/mL, and may be used in applications where inhibition of RNase activity is desired.

In another embodiment there is provided an aqueous solution comprising PVSA in a concentration range of from 0.1 to 2000 μg/mL, such as from 0.1 to 1000 μg/mL, as from 0.1 to 500 μg/mL, such as from 0.1 to 200 μg/mL, such as from 0.1 to 180 μg/mL, such as from 0.1 to 150 μg/mL, such as from 0.1 to 120 μg/mL, such as from 0.1 to 100 μg/mL, such as from 0.1 to 90 μg/mL.

In an embodiment such an aqueous solution further comprises an ionic, zwitter-ionic or non-ionic detergent, in particular a non-ionic detergent. Non-ionic detergents may be selected from the group comprising Triton x100, SDS, NP-40/Igepal, Sarkosyl, Tween-20, sodium deoxycholate, and CHAPS.

Aqueous solutions of PVSA may be in a concentration from 10 to 2000 μg/mL, such as from 10 to 1000 μg/mL, such as from 10 to 500 μg/mL, such as from 10 to 200 μg/mL.

Aqueous solutions of PVSA may further comprise dNTPs and oligo dT primers.

Alternatively, aqeuous solutions may consist only of PVSA and water. In such solutions the concentration of PVSA may be from 10 to 2000 μg/mL, such as from 10 to 1000 μg/mL, such as from 10 to 500 μg/mL, such as from 10 to 200 μg/mL.

Aqueous solutions of PVSA and optionally other components may be used as RNase inhibitors in cell lysis. The cell lysis may be a step in RNA sequencing, such as single-cell RNA sequencing. The concentration of PVSA when used in a cell lysis buffer may be from 0.1 to 120 μg/mL, such as from 15 to 120 μg/mL, such as from 30 to 100 μg/mL, such as from 40 to 80 μg/mL, such as from 50 to 70 μg/mL, such as 60 μg/mL. The cell lysis may be a step in single cell RNA-sequencing.

Alternatively, the concentration of PVSA may be from 0.1 to 30 μg/mL, such as from 0.1 to 20 μg/mL, such as from 0.3 to 10 μg/mL, such as from 0.3 to 15 μg/mL, such as from 0.3 to 5 μg/mL, such as from 0.6 to 15 μg/mL, such as from 0.6 to 6 μg/mL, such as from 1.5 to 6 μg/mL, such as from 0.5 to 10 μg/mL, such as from 0.6 to 9 μg/mL, such as from 1 to 8 μg/mL, such as from 3 to 8 μg/mL, such as from 4 to 7 μg/mL, such as 6 μg/mL. The cell lysis may be a step in single cell RNA sequencing.

Single-cell RNA sequencing may be according to different protocols available to the skilled person. Many such protocols are known. An example of the use according to the present invention is the use of the Smart-seq2 protocol. An example of use of the Smart-seq2 protocol comprises:
a) isolating a single cell into a lysis buffer comprising a detergent, dNTPs, Smart-seq2 oligo-dT primer, and an aqueous solution of PVSA);
b) lysing the cell and annealing said Smart-seq2 oligo-dT primer in one step;
c) adding reverse transcription mix (1× reverse transcriptase, DTT, betaine, mM MgCl₂, Smart-seq2 TSO, and Superscript II);
d) generating first-strand cDNA;
e) adding an amplification mix (KAPA HiFi HotStart Ready Mix and Smart-seq2 ISPCR primers) to the first-strand cDNA;
f) amplifying the cDNA
g) optionally tagmenting the cDNA using Tn5 enzyme and
h) optionally indexing/barcoding the cDNA to create sequencing libraries.

The detergent comprised in the lysis buffer of step 1 may be Triton X-100 and the concentration of Triton X-100 may be 0.1% (v/v); the concentration of dNTPs may be 2.5 mM; the concentration of Smart-seq2 oligo-dT primer may be 2.5 mM.

The concentration of DTT in the reverse transcription mix of step 3 may be 5 mM, the concentration of betaine in said mix may be 1M, the concentration of MgCl₂ in said mix may be 10 mM, the concentration of Smart-Seq2 TSO in said mix may be 1 mM; and the reverse transcriptase may be 10 U of Superscript II in said mix. The concentration of Smart-seq2 ISPCR primers in the amplification mix of step 4 may be 80 nM.

Another example according to the invention is the use of the Smart-Seq3 protocol. An example of the use of the Smart-Seq3 protocol comprises:
a) isolating a single cell into a lysis buffer comprising a detergent, PEG 8000, dNTPs, and Smart-seq3 oligo-dT primer, and an aqueous solution of PVSA);
b) lysing the cell and annealing said Smart-seq3 oligo-dT primer in one step;
c) adding reverse transcription mix (Tris buffer, DTT, MgCl₂, Smart-seq3 TSO, and reverse transcriptase);
d) generating first-strand cDNA;
e) adding an amplification mix (Kapa Hifi buffer and polymerase, dNTPs, Smart-seq3 forward amplification primers and Smart-seq3 reverse amplification primers) to the first-strand cDNA;
f) amplifying the cDNA
g) optionally tagmenting the cDNA using Tn5 enzyme and
h) optionally indexing/barcoding the cDNA to create sequencing libraries.

The detergent may be Triton X-100 and the concentration of Triton X-100 may be 0.1% (v/v); the concentration of dNTPs may be 1 mM/ea; the concentration of PEG 8000 may be 10% (w/v); and the concentration of Smart-seq3 oligo-dT primer is 1 mM.

The concentration of Tris-HCl in the reverse transcription mix may be 50 mM, concentration of DTT in the reverse transcription mix of step 3 may be 8 mM, the concentration of MgCl2 in said mix may be 5 mM, the concentration of Smart-seq3 TSO in said mix may be 2 M; and the reverse transcriptase may be 8 U of Maxima H-minus enzyme in said mix.

The concentration of Smart-seq3 forward amplification primers may be 500 nM and reverse amplification primers may b 100 nM in the amplification mix of step 4.

Another example according to the invention is the use of the Smart-Seq3Express protocol. An example of the use of the Smart-Seq3Express protocol comprises:
a) isolating a single cell into a lysis buffer comprising a detergent, PEG 8000, dNTPs, and Smart-seq3 oligo-dT primer, and an aqueous solution of PVSA;
b) lysing the cell and annealing said Smart-seq2 oligo-dT primer in one step;
c) adding reverse transcription mix (Tris buffer, DTT, MgCl₂, Smart-seq3 TSO, and reverse transcriptase);
d) generating first-strand cDNA;
e) adding an amplification mix (SeqAmp PCR buffer and SeqAmp DNA polymerase, Smart-seq3 forward amplification primers and Smart-seq3 reverse amplification primers) to the first-strand cDNA;
f) amplifying the cDNA
g) optionally tagmenting the cDNA using Tn5 enzyme and
h) optionally indexing/barcoding the cDNA to create sequencing libraries.

In such a protocol RNA-sequencing the detergent comprised in the lysis buffer of step 1 may be Triton X-100 and the concentration of Triton X-100 may be 0.1% (v/v); the concentration of dNTPs may be 0.5 mM/ea; the concentration of PEG 8000 may be 5% (w/v); and the concentration of Smart-seq3 oligo-dT primer may be 0.125 M.

In such a protocol the concentration of Tris-HCl in the said mix may be 25 mM, concentration of DTT in the reverse transcription mix of step 3 may be 8 mM, the concentration of MgCl2 in said mix may be 2.5 mM, the concentration of Smart-seq3 TSO in said mix may be 0.75 M; and the reverse transcriptase may be 2 U of Maxima H-minus enzyme in said mix.

In such a protocol the concentration Smart-seq3 forward reverse amplification primers may be 500 nM and the concentration of reverse amplification primers may be 500 nM in the amplification mix of step 4.

The invention will now be described by reference to the following Figures and Examples.

### FIGURES

FIG. 1. Replacing recombinant RNase inhibitor with PVSA in RNA-seq library generation.

(a) Molecular structure of PVSA. (b) Bioanalyzer traces of Smart-seq2 (SS2) cDNA libraries generated from 100 pg mouse RNA using varying PVSA concentration (0-360 μg/mL) or standard recombinant RNase inhibitor (SS2-RI) in lysis buffer. (c) Average cDNA yield (dot) and standard error (whiskers) of SS2 cDNA libraries (integration range 200-10,000 bp). (d) Average percent (dot) and range (whiskers) of primer-dimer in SS2 cDNA libraries (integration range 20-50 bp). (e) Electrophoresis agarose gel images of PCR products formed using eGFP plasmid (left) or human gDNA (right) as template at varying concentrations of PVSA in PCR, demonstrating variable amounts of on and off-target products. In case of gDNA, five different primer-probe pair (P1-5) were used targeting the Tubala gene. Data shown as median, interquartile range (IQR), and 1.5×IQR. (f) Box plot of percent uniquely mapped sequencing reads to the reference genome. (g) Box plot of number of genes detected for each inhibitor condition. (h) Stacked bar plot of fraction reads mapping to intronic, exonic, or intergenic regions of the mouse genome. (i) Histogram of fraction of bases along sequencing reads matching the genome, indicating read quality. (j) Normalized gene body coverage of mapped reads along transcripts.

FIG. 2. Performance of PVSA in single-cell RNA-seq.

(a) Bioanalyzer traces of SS2 cDNA libraries from HEK293FT single cells using varying PVSA concentrations (0-480 μg/mL) or standard recombinant RNase inhibitor (SS2-RI) in lysis buffer. (b) Average cDNA yield (dot) and standard error (whiskers) of HEK293FT SS2 cDNA libraries (integration range 200-10,000 bp). (c) Average percent (dot) and range (whiskers) of primer-dimer in HEK293FT SS2 cDNA libraries (integration range 20-50 bp). (d) Box plot of number of genes detected in HEK293FT cell libraries. (e) Stacked bar plot of fraction reads mapping to intronic, exonic, or intergenic regions of the human genome for HEK293FT cell libraries. (f) Normalized gene body coverage of mapped in HEK293FT cell libraries. (g, h, i) Uniform manifold approximation and projection (UMAP) for mouse liver and kidney-derived cells, coloured by (g) source tissue, (h) RNase inhibitor condition [200, 300 μg/mL PVSA or standard SS2 with recombinant inhibitor], (i) cell cluster based on transcriptome signature. (j-m) Mouse liver kidney-cell UMAP coloured by expression level of top-variable genes of four cell clusters: Cd79a, Trbc1, Ahsg, and Lyz1. (n) Expression-level heatmap of 10 top-variable genes for each UMAP-generated cluster. (o) Bioanalyzer traces of Smart-seq3 (SS3) cDNA libraries generated from 100 pg mouse RNA using varying PVSA concentration (0-90 μg/mL) or standard recombinant RNase inhibitor (SS3-RI) in lysis buffer. (p) Average cDNA yield (dot) and standard error (whiskers) of SS3 100 pg bulk cDNA libraries (integration range 200-10,000 bp). (q) Bioanalyzer traces of SS3 cDNA libraries from HEK293FT single cells using varying PVSA concentration (1.5, 3, 6 μg/mL) or standard recombinant RNase inhibitor (SS3-RI) in lysis buffer. (r) Average cDNA yield (dot) and standard error (whiskers) of SS3 HEK293FT single cells libraries. (s) Box plot of number of genes detected in SS3 libraries of different inhibitor conditions for 100 pg mouse bulk RNA (left) and HEK293FT single cells (right). (t) Box plot of unique molecular identifiers (UMIs) detected in SS3 libraries of different inhibitor conditions for 100 pg mouse bulk RNA (left) and HEK293FT single cells (right). (u) Stacked bar plot of fraction reads mapping to intronic, exonic, or intergenic regions of the human genome for SS3 HEK293FT single cells of different inhibitor condition. (v) Normalized gene body coverage of mapped reads along transcripts for SS3 HEK293FT single cells, with characteristic 5′-bias towards the UMI end.

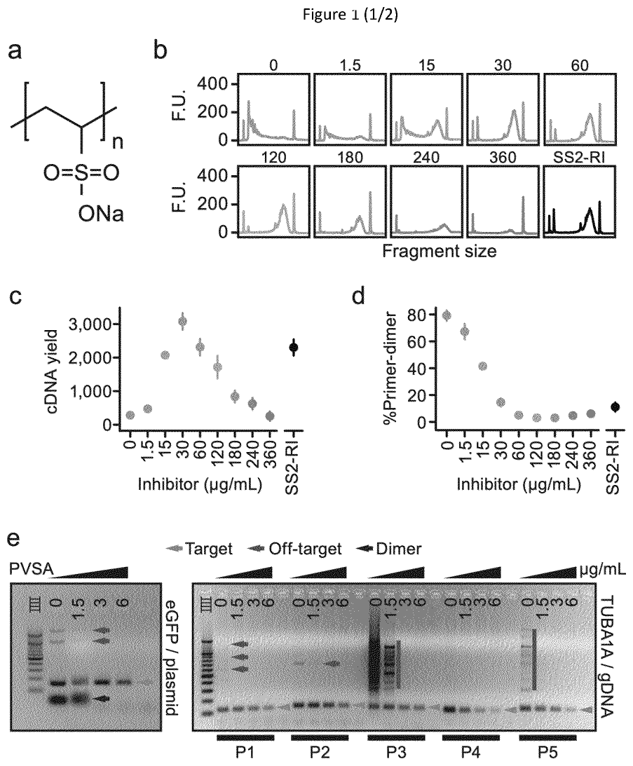

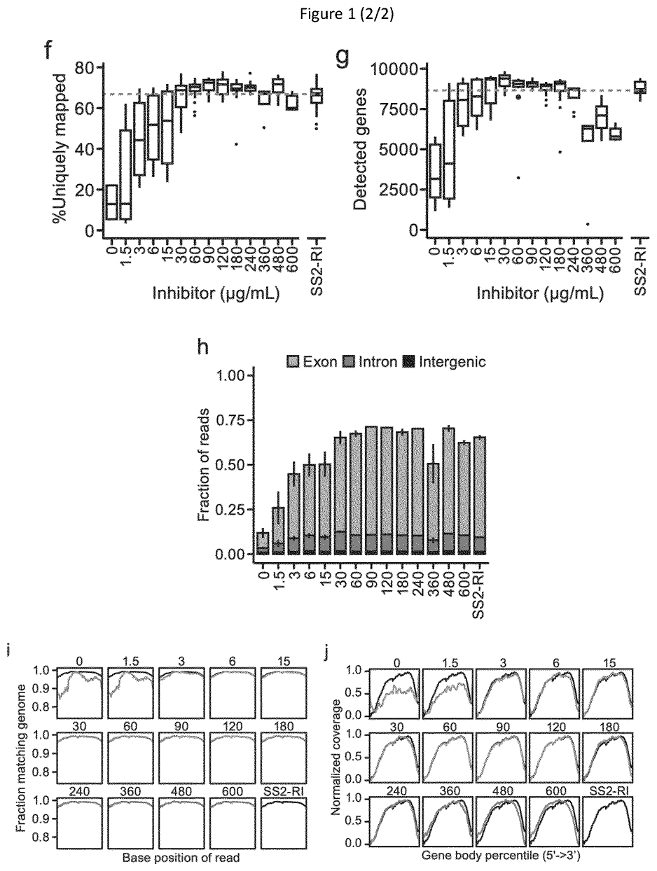

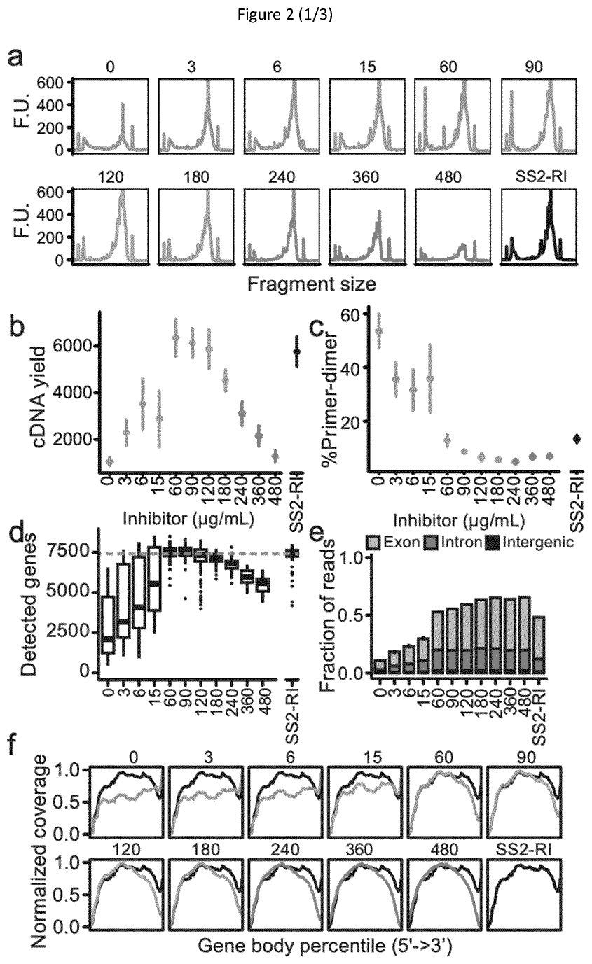

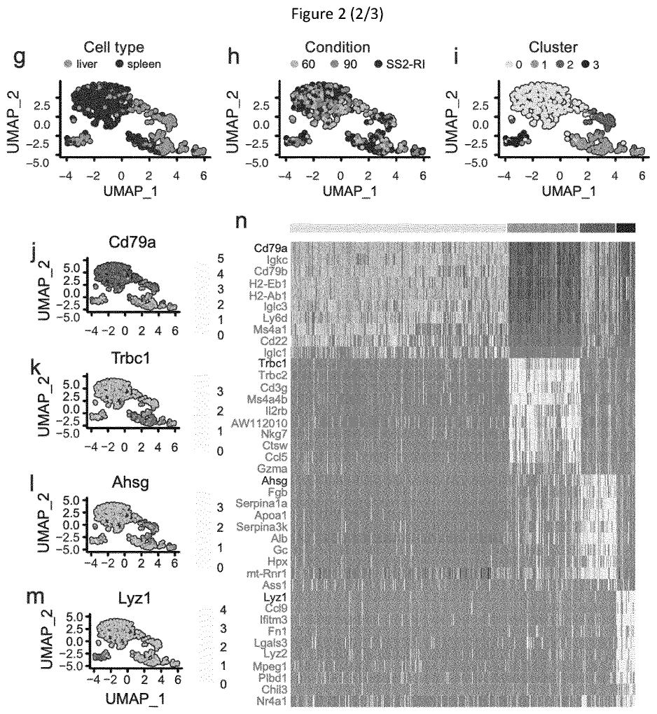

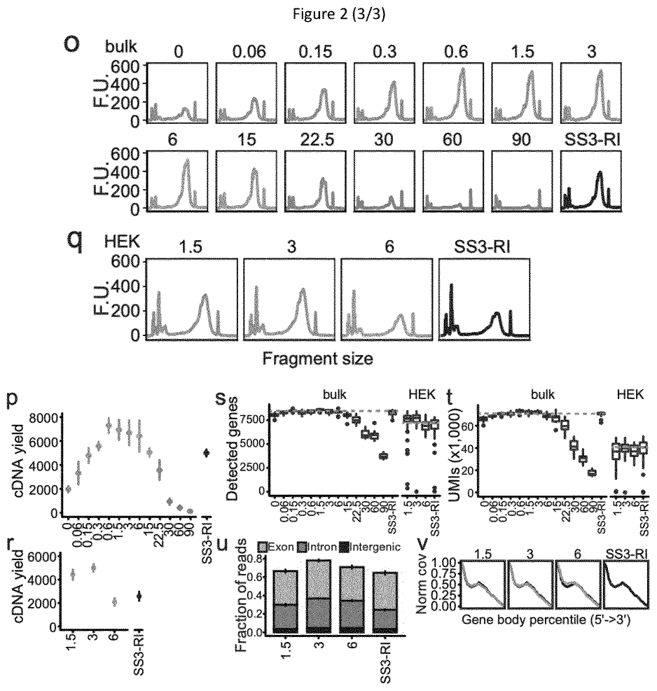

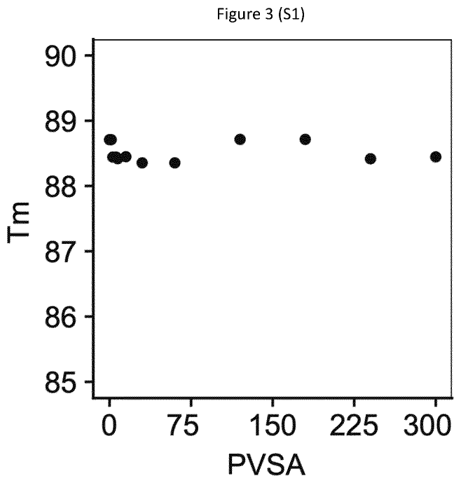

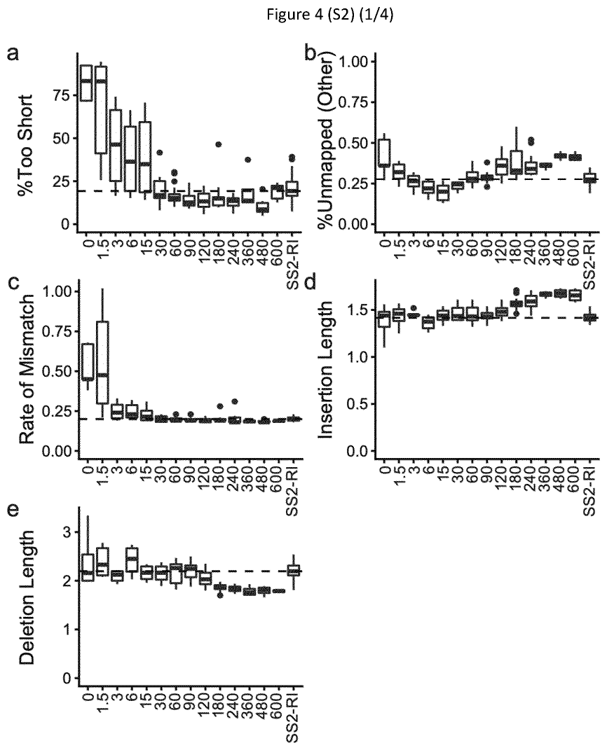

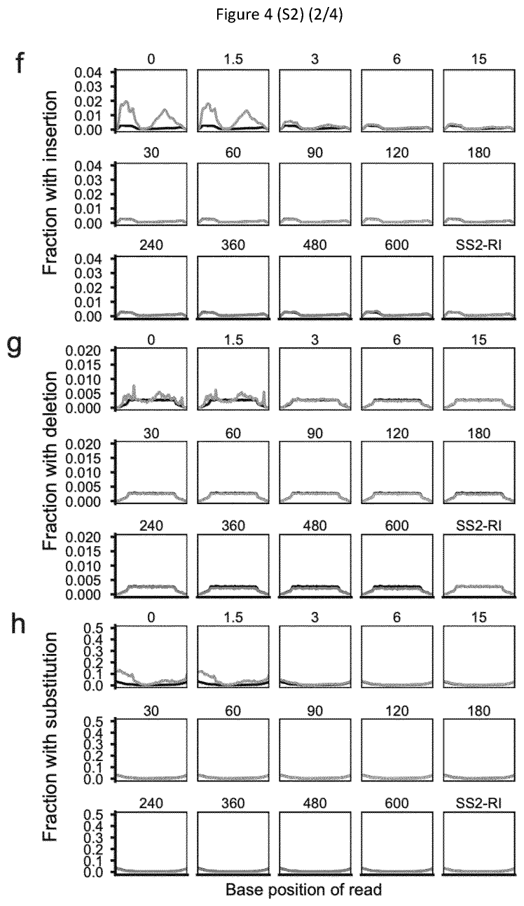

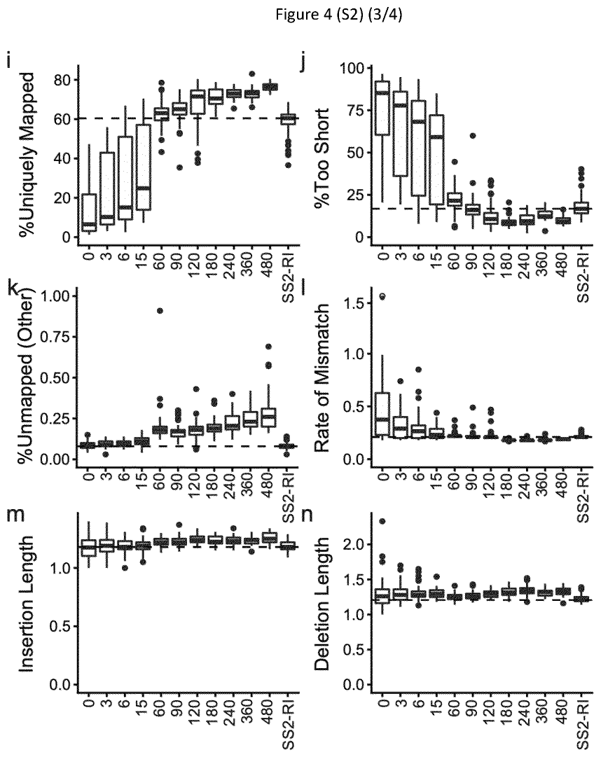

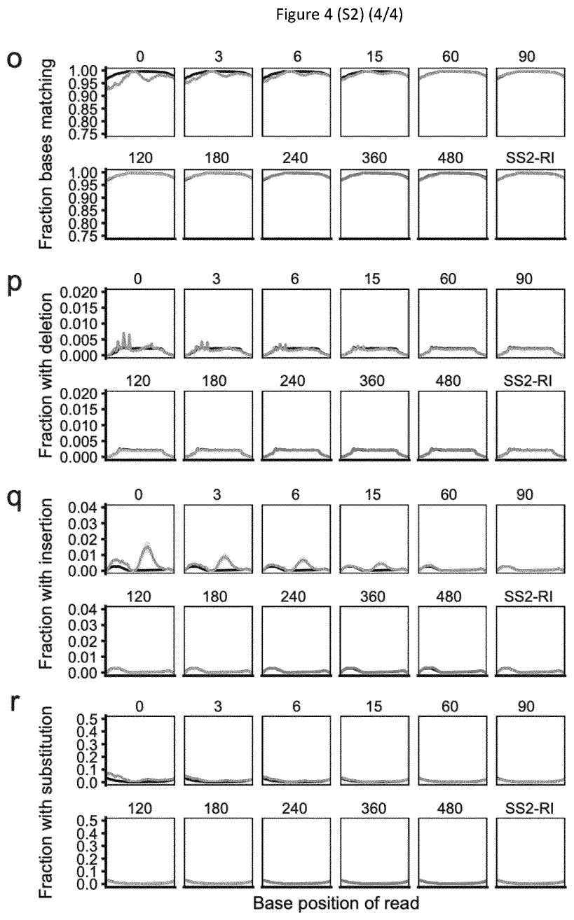

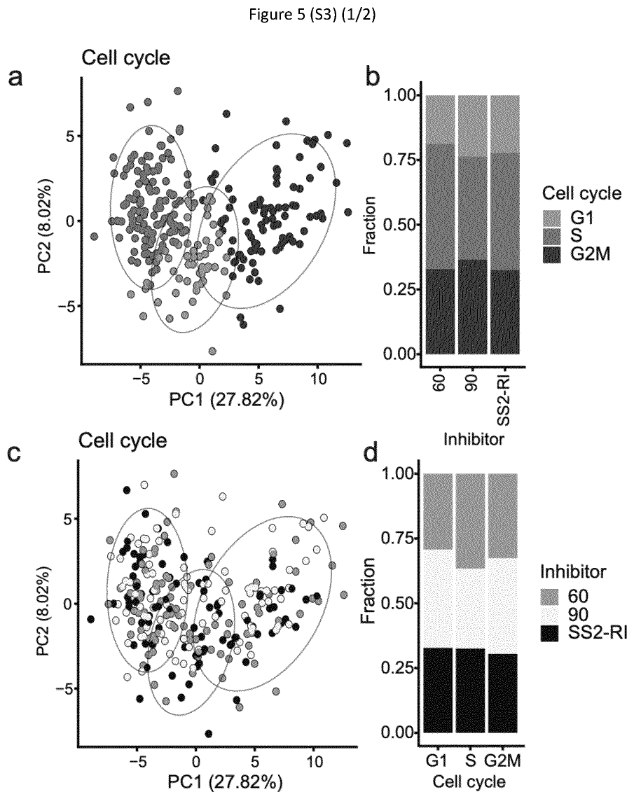

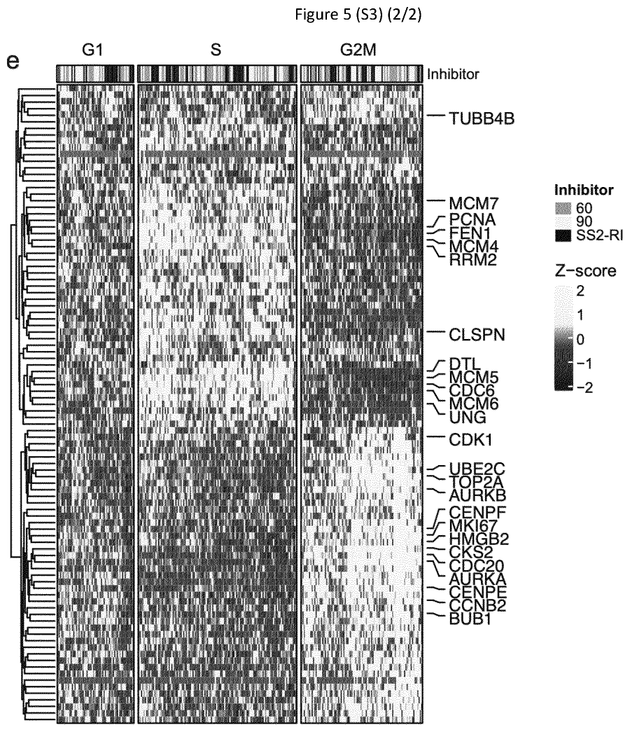

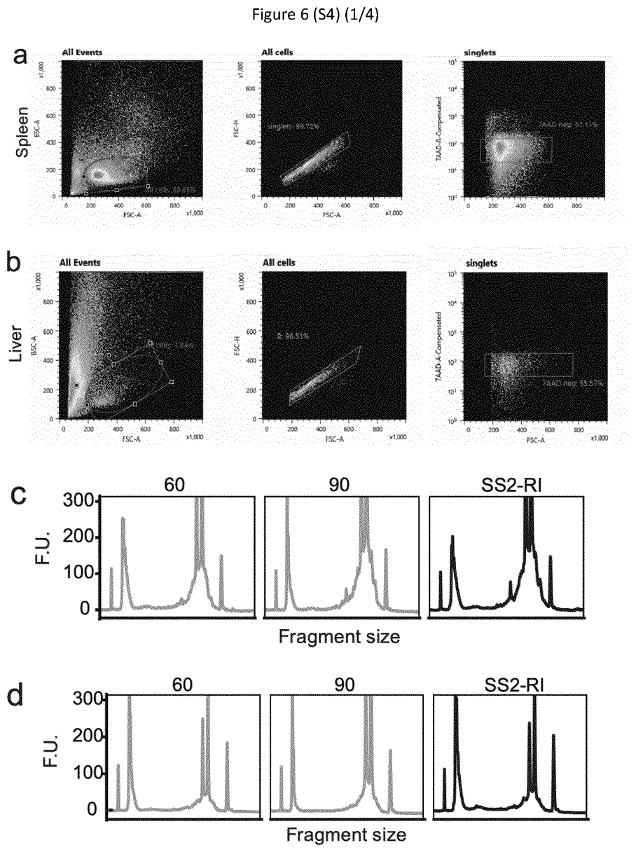

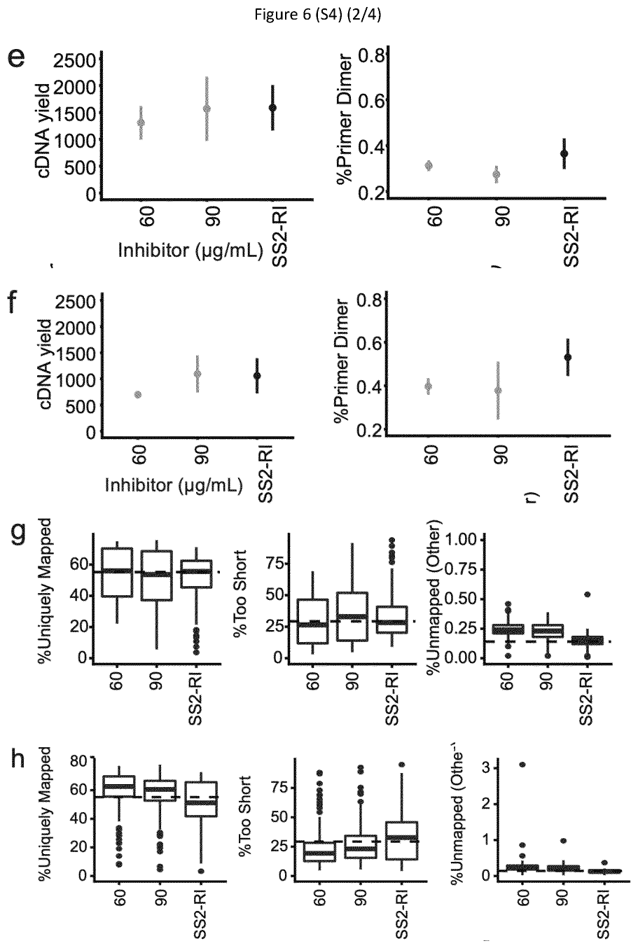

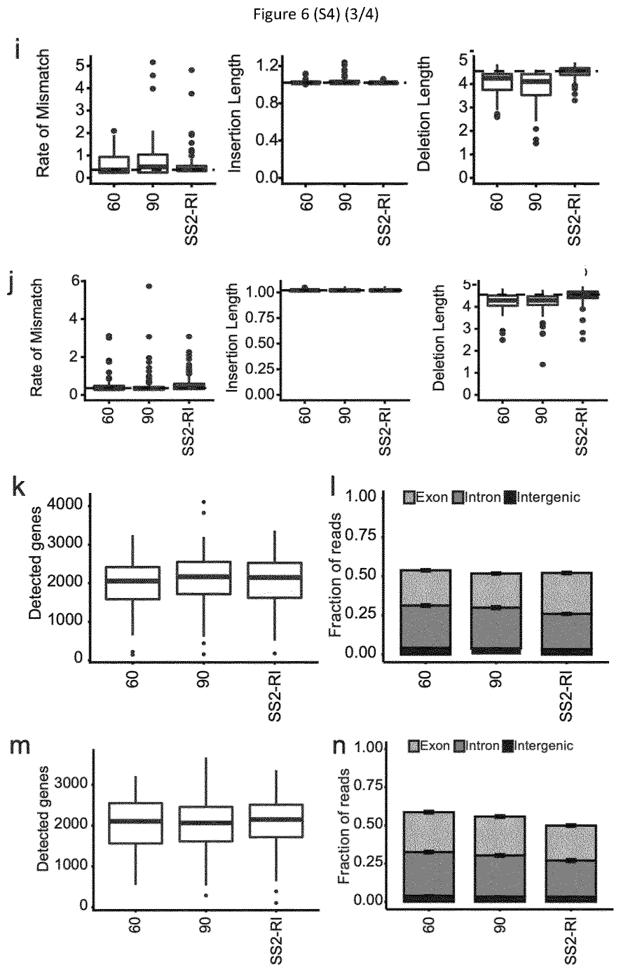

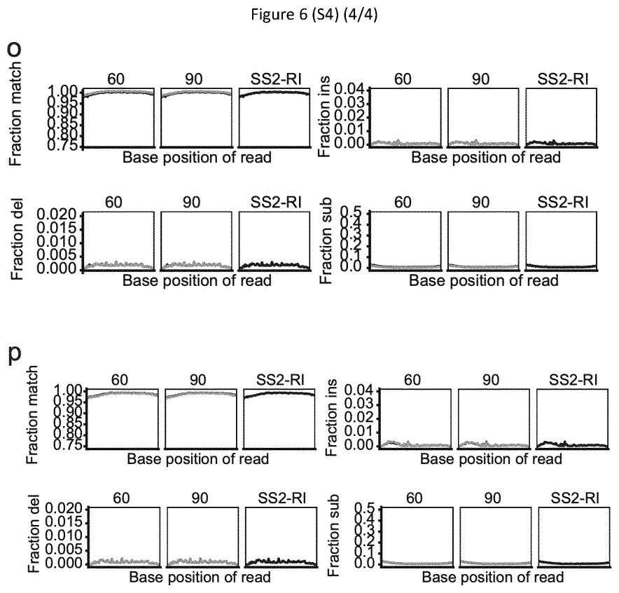

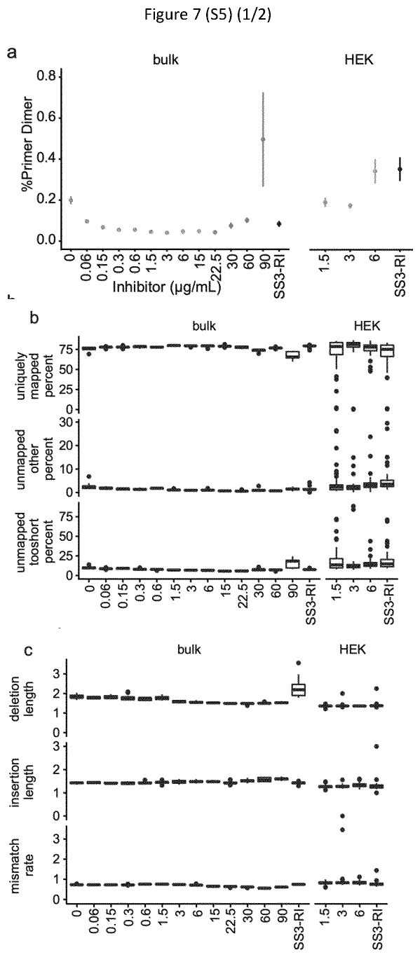

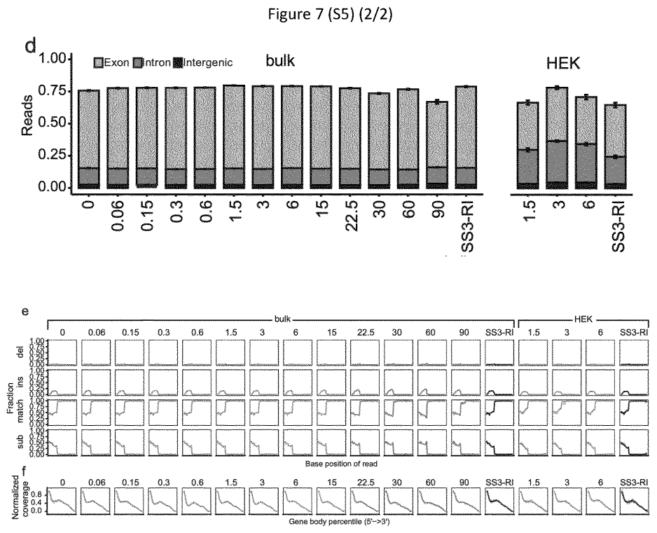

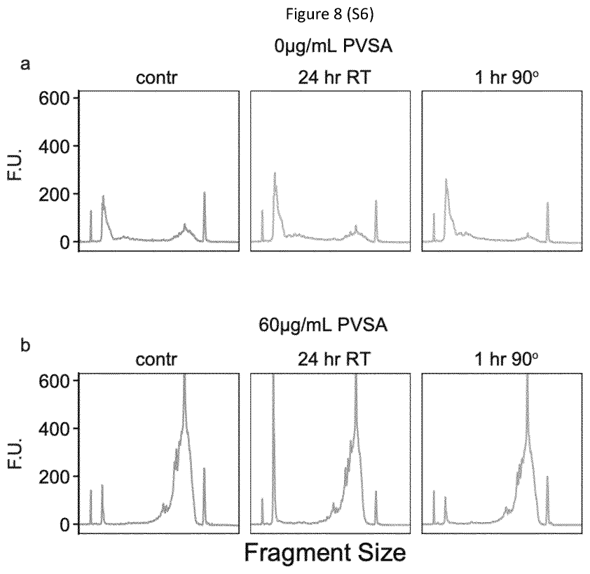

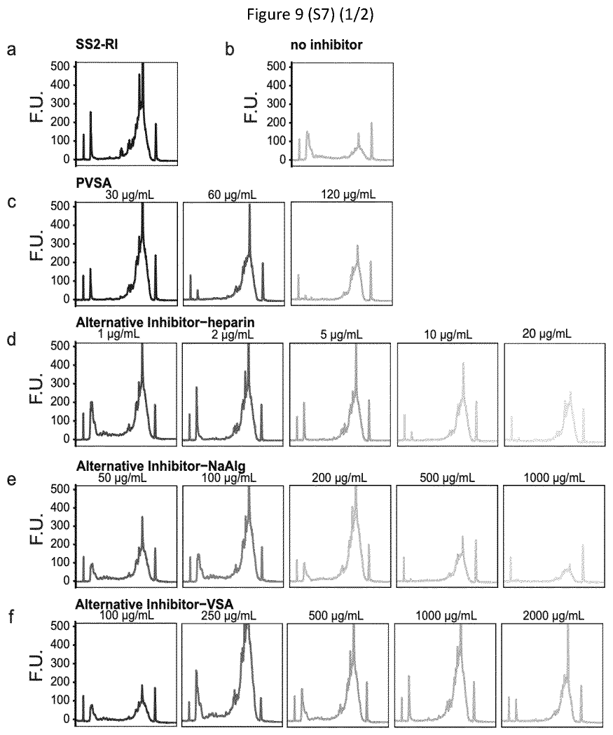

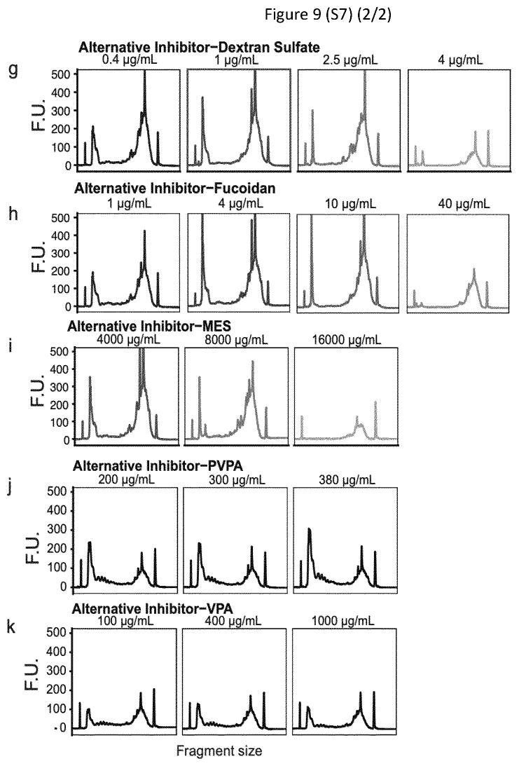

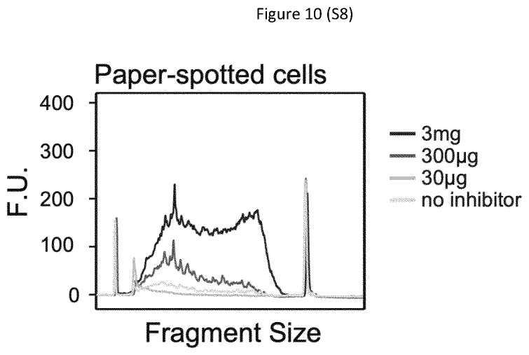

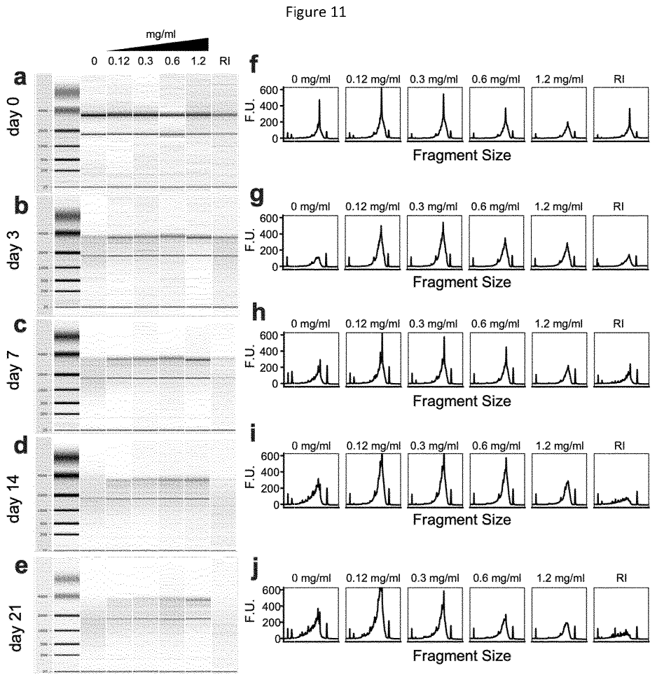

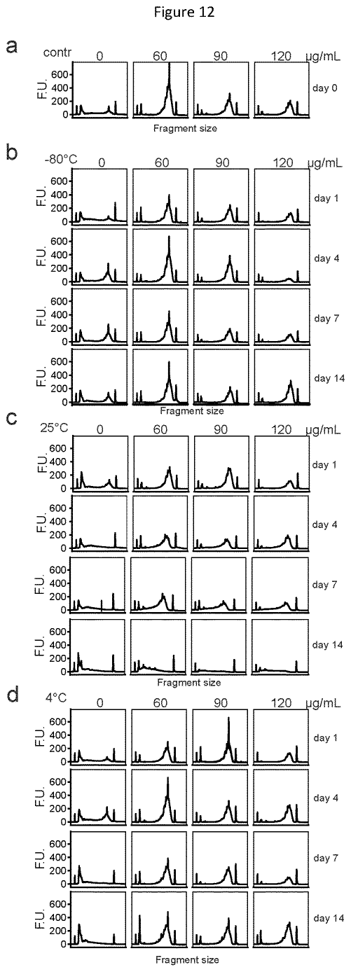

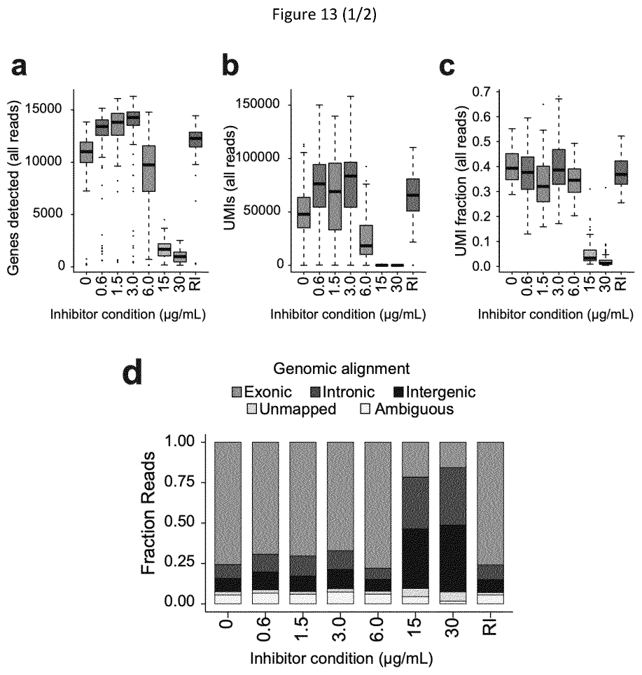

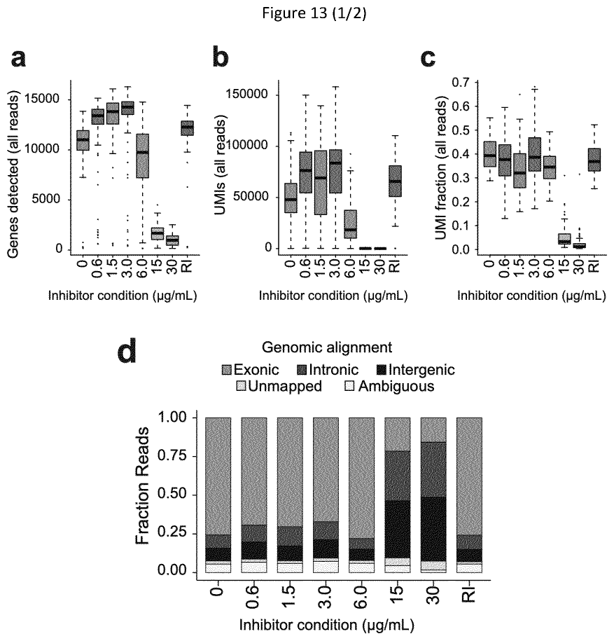

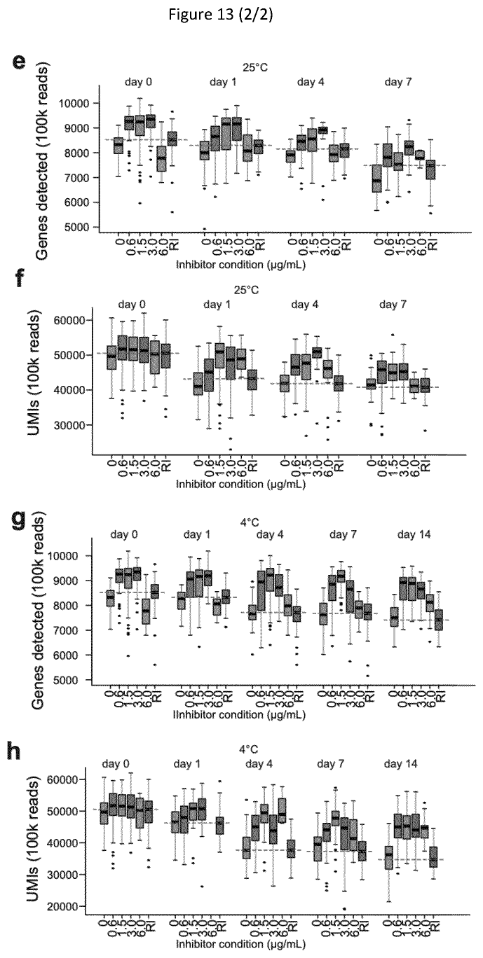

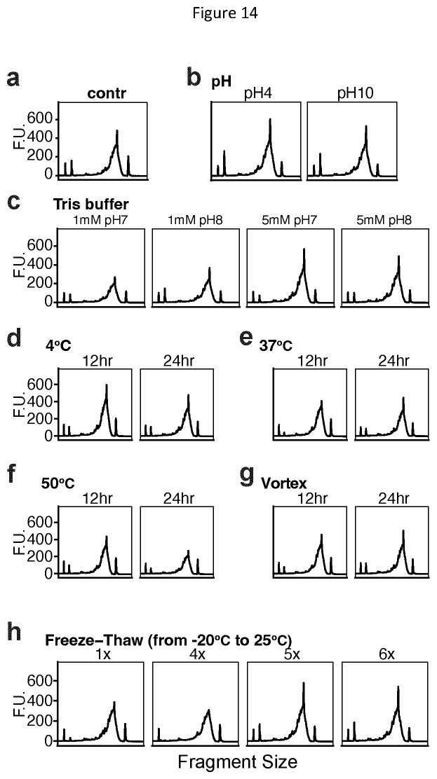

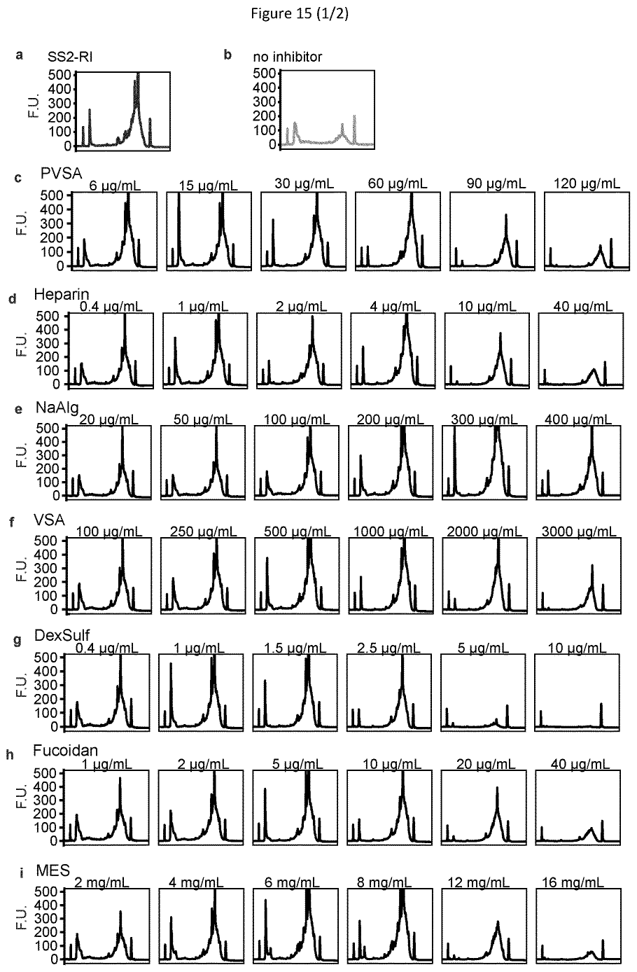

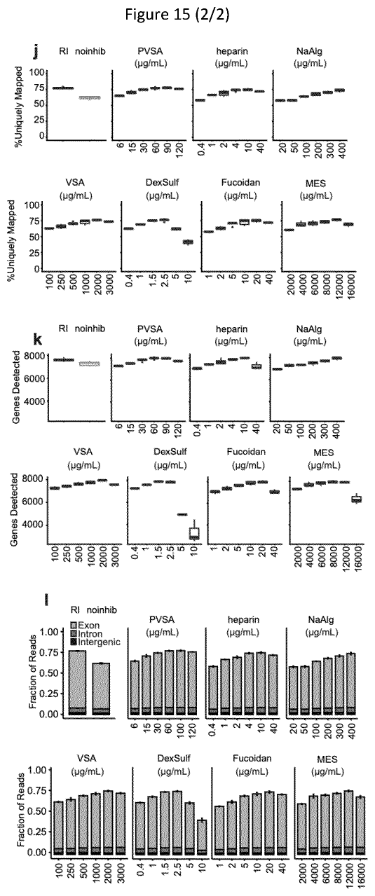

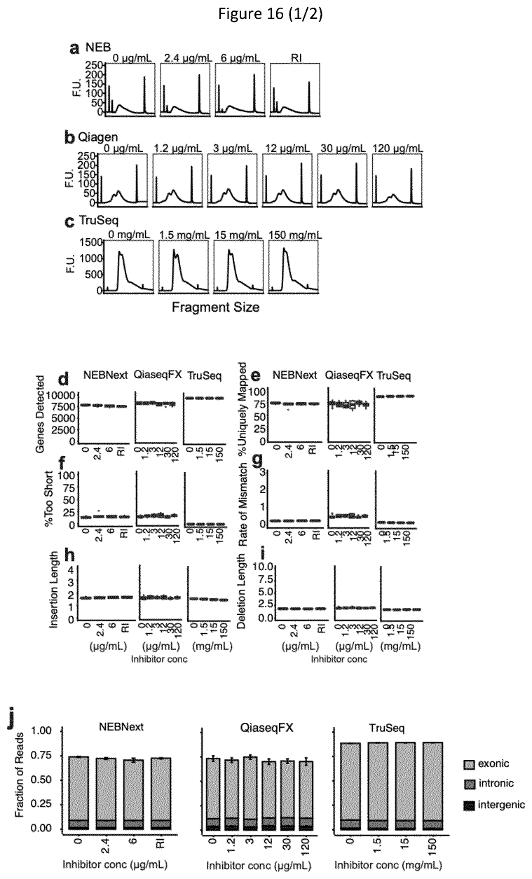

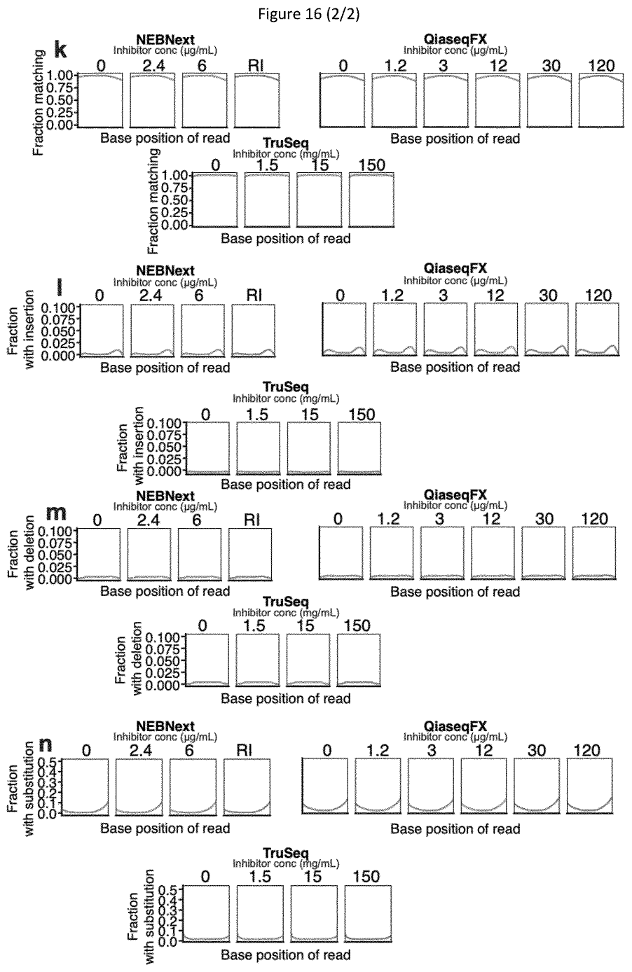

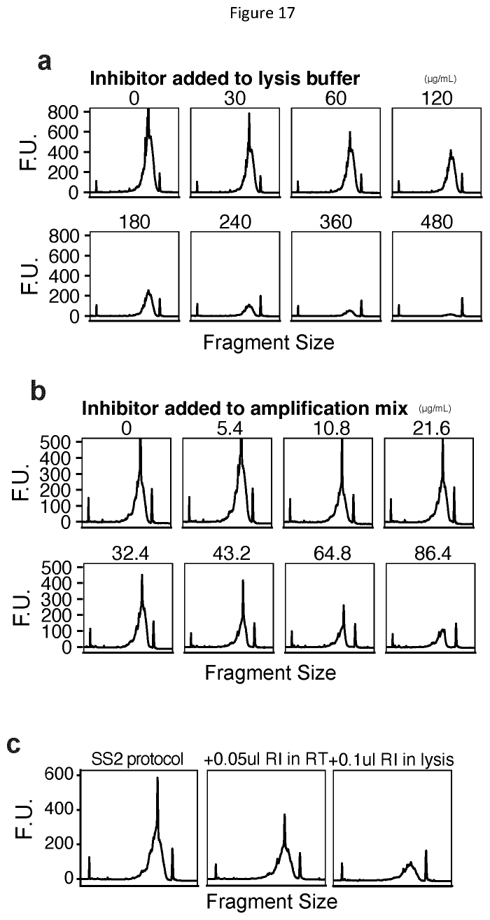

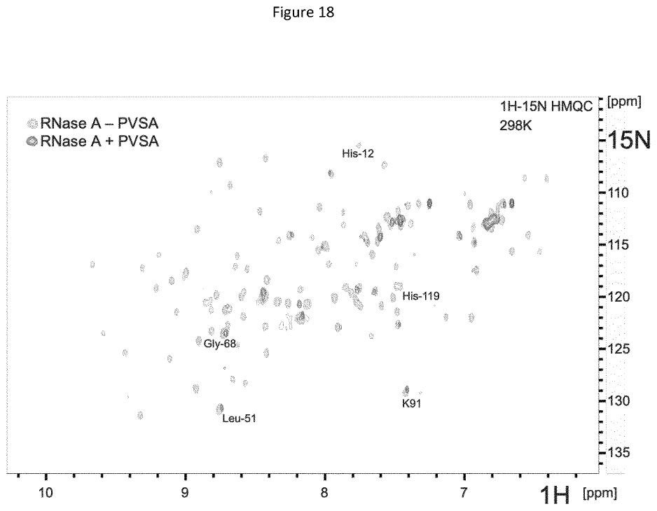

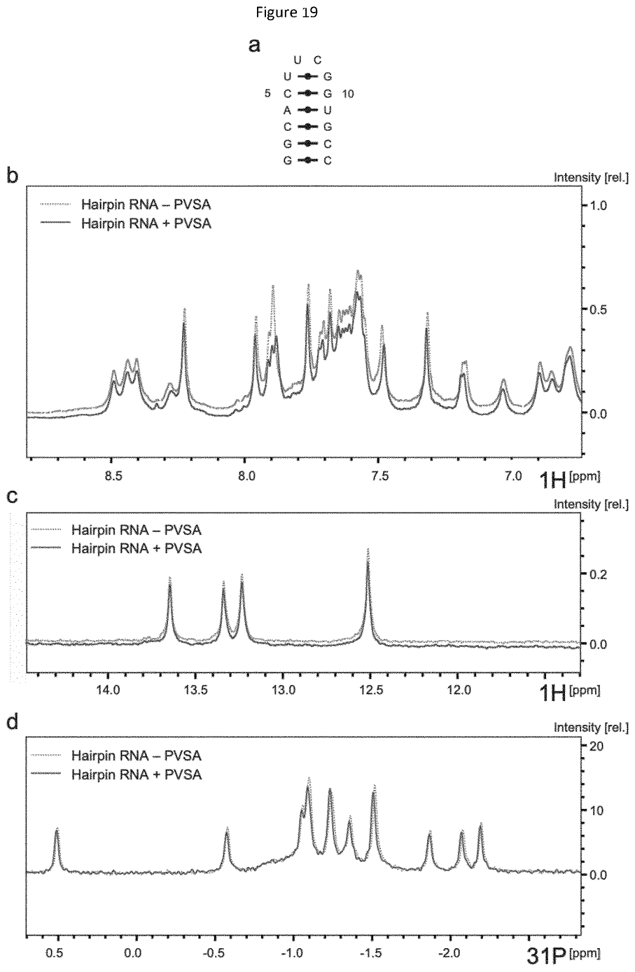

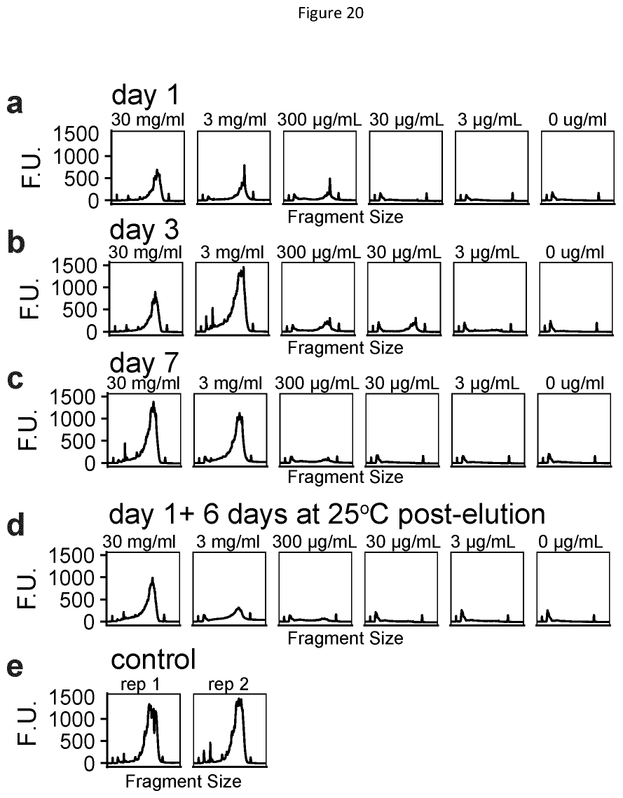

FIG. 3 (Supplementary FIG. 1). Melting temperature analysis of PVSA-containing samples.

Comparison of the melting temperatures of a 164 bp DNA duplex in various concentrations of PVSA (1-300 μg/mL) via denaturation detection with Sybr Green fluorescence.

FIG. 4 (Supplementary FIG. 2). QC for SS2 bulk and HEK cell libraries.

(a) Box plot of percent sequencing reads too short to be mapped to the reference genome for SS2 bulk samples. (b) Box plot of percent sequencing reads unmapped (other) to the reference genome for SS2 bulk samples. (c) Box plot of the average insertion length for SS2 bulk samples (d) Box plot of the mismatch rate length for SS2 bulk samples. (e) Box plot of the average deletion length for SS2 bulk samples. (f) Histogram of fraction of bases along sequencing reads with insertion for SS2 bulk samples. (g) Histogram of fraction of bases along sequencing reads with deletion for SS2 bulk samples. (h) Histogram of fraction of bases along sequencing reads with substitution for SS2 bulk samples. (i) Box plot of percent sequencing reads too short to be mapped to the reference genome for SS2 HEK samples. (j) Box plot of percent sequencing reads unmapped (other) to the reference genome for SS2 HEK samples. (k) Box plot of the mismatch rate length for SS2 bulk samples. (1) Box plot of the average insertion length for SS2 HEK samples (m) Box plot of the average deletion length for SS2 HEK samples. (n) Histogram of fraction of bases along sequencing reads with insertion for SS2 HEK samples. (o) Histogram of fraction of bases along sequencing reads with deletion for SS2 HEK samples.

FIG. 5 (Supplementary FIG. 3). Cell cycle analysis of HEK cells.

(a) PCA plot for cell cycle genes with cells from HEK cells using samples with 60 μg/mL PVSA, 90 μg/mL PVSA, or recombinant RNase inhibitor (SS2-RI) in lysis buffer colored according to cell cycle phase. (b) Bar plot for fraction of cells in each cell cycle phase for each condition. (c) PCA plot for cell cycle genes with cells from HEK cells using samples with 60 μg/mL PVSA, 90 μg/mL PVSA, or SS2-RI in lysis buffer colored according to condition. (d) Bar plot for fraction of cells in each condition for each cell cycle phase. (e) Expression-level heatmap of highly variable cell cycle genes grouped by phase for conditions 60 μg/mL PVSA, 90 μg/mL PVSA, and SS2-RI.

FIG. 6 (Supplementary FIG. 4). FACS gate, cDNA library characteristics and QC of liver and spleen cells.

(a) FACS plots of spleen cells gating for lymphocyte population (left), singlets (middle), and viable cells (7AADneg) (right). (b) FACS plots of liver cells gating for lymphocyte population (left), singlets (middle), and viable cells (7AAD-neg) (right). (c) Bioanalyzer traces of Smart-seq2 (SS2) cDNA libraries generated from sorted spleen cells using 60 μg/mL PVSA, 90 μg/mL PVSA, or standard recombinant RNase inhibitor (SS2-RI) in lysis buffer. (d) Bioanalyzer traces of Smart-seq2 (SS2) cDNA libraries generated from sorted liver cells using 60 μg/mL PVSA, 90 μg/mL PVSA, or standard recombinant RNase inhibitor (SS2-RI) in lysis buffer. (e) Average cDNA yield (dot) and standard error (whiskers) of SS2 cDNA libraries from spleen (integration range 200-10,000 bp) (left) and average percent (dot) and range (whiskers) of primer-dimer in SS2 cDNA libraries from spleen (integration range 20-50 bp) (right). (f) Average cDNA yield (dot) and standard error (whiskers) of SS2 cDNA libraries from liver (integration range 200-10,000 bp) (left) and average percent (dot) and range (whiskers) of primer-dimer in SS2 cDNA libraries from liver (integration range 20-50 bp) (right). (g) Box plots of percent uniquely mapped (left), percent sequencing reads too short to be mapped (middle), and percent sequencing reads unmapped (not due to fragments too short or too many mismatches) (right) to the reference genome for spleen cells. (h) Box plots of percent uniquely mapped (left), percent sequencing reads too short to be mapped (middle), and percent sequencing reads unmapped (not due to fragments too short or too many mismatches) (right) to the reference genome for liver cells. (i) Box plot of rate of mismatch per base (left), average insertion length (middle), and average deletion length (right) for spleen cells. (j) Box plot of rate of mismatch per base (left), average insertion length (middle), and average deletion length (right) for liver cells. (k) Box plot of number of genes detected for each inhibitor condition for spleen cells. (l) Stacked bar plot of fraction reads mapping to intronic, exonic, or intergenic regions of the mouse genome for spleen cells. (m) Box plot of number of genes detected for each inhibitor condition for liver cells. (n) Stacked bar plot of fraction reads mapping to intronic, exonic, or intergenic regions of the mouse genome for liver cells. (o) Histogram of fraction of bases along sequencing reads matching the genome (top left), with an insertion (top right), with a deletion (bottom left), and with a substitution (bottom right) indicating read quality for spleen cells. (p) Histogram of fraction of bases along sequencing reads matching the genome (top left), with an insertion (top right), with a deletion (bottom left), and with a substitution (bottom right) indicating read quality for liver cells. (q) Normalized gene body coverage of mapped reads along transcripts for spleen cells. (r) Normalized gene body coverage of mapped reads along transcripts for liver cells.

FIG. 7 (Supplementary FIG. 5). QC for SS3 bulk and HEK cell libraries.

(a) Average percent (dot) and range (whiskers) of primer-dimer in SS3 cDNA libraries from mouse RNA and HEK cells (integration range 20-50 bp). (b) Box plots of percent uniquely mapped (top), percent sequencing reads too short to be mapped (middle), and percent sequencing reads unmapped (not due to fragments too short or too many mismatches) (bottom) to the reference genome for mouse RNA and HEK cells. (c) Box plot of average insertion length (top), average deletion length (middle), and rate of mismatch per base (bottom) for mouse RNA and HEK cells. (d) Stacked bar plot of fraction reads mapping to intronic, exonic, or intergenic regions of the mouse genome for mouse RNA and HEK cells. (e) Histogram of fraction of bases along sequencing reads with a deletion, with an insertion, matching the genome, and with a substitution indicating read quality for mouse RNA and HEK cells. (f) Normalized gene body coverage of mapped reads along transcripts for mouse RNA and HEK cells.

FIG. 8 (Supplementary FIG. 6). Thermostability RNase inhibitor PVSA.

(a) Bioanalyzer traces of Smart-seq2 (SS2) cDNA libraries generated from 100 pg mouse RNA using 0 μg/mL PVSA following standard protocol (left), incubating lysis buffer+RNA for 24 hours at room temperature (middle), and incubating lysis buffer+RNA for 1 hr at 90° C. (right). (b) Bioanalyzer traces of Smart-seq2 (SS2) cDNA libraries generated from 100 pg mouse RNA using 60 μg/mL PVSA following standard protocol (left), incubating lysis buffer+RNA for 24 hours at room temperature (middle), and incubating lysis buffer+RNA for 1 hr at 90° C. (right).

FIG. 9 (Supplementary FIG. 7). cDNA library generation using various polymers as RNA inhibitors.

(a) Bioanalyzer trace of Smart-seq2 (SS2) cDNA libraries generated from 100 pg mouse RNA with standard biological inhibitor. (b) Bioanalyzer trace of SS2 cDNA library without inhibitor. (c) Bioanalyzer traces of SS2 cDNA library with PVSA (30-120 μg/mL). (d) Bioanalyzer traces of SS2 cDNA library with heparin (1-20 μg/mL). (e) Bioanalyzer traces of SS2 cDNA library with sodium alginate (50-1000 μg/mL). (f) Bioanalyzer traces of SS2 cDNA library with vinylsulfonic acid (VSA) (100-2000 μg/mL). (g) Bioanalyzer traces of SS2 cDNA library with dextran sulfate (0.4-4 μg/mL). (h) Bioanalyzer traces of SS2 cDNA library with fucoidan (1-40 μg/mL). (i) Bioanalyzer traces of SS2 cDNA library with 2-(N-morpholino)ethanesulfonic acid (MES) (4000-16000 μg/mL). (j) Bioanalyzer traces of SS2 cDNA library with polyvinyl phosphonic acid (PVPA) (200-380 μg/mL). (k) Bioanalyzer traces of SS2 cDNA library with vinylphosphonic acid (VPA) (100-1000 μg/mL).

FIG. 10 (Supplementary FIG. 8). cDNA yield from paper material coated with polyvinyl sulfonic acid.

Bioanalyzer traces of Smart-seq2 cDNA libraries generated from paper-eluted liver cells preincubated in PVSA (30 μg/mL, 300 μg/mL, 10 mg/mL) or water only (no inhibitor). FU: Fluorescence Units.

FIG. 11. RNA storage buffer capacity with the addition of PVSA.

RNA Bioanalyzer traces of 20 ng of HEK293FT RNA stored at room temperature in storage buffer containing either 0, 0.12, 0.3, 0.6, 1.2 mg/mL PVSA, or 10 U/μl recombinant RNase inhibitor (RI) (left) showing ribosomal bands S28 and S1,8 and corresponding bulk-RNA Smart-seq2 (SS2) cDNA traces from cDNA generated with 8 ng of RNA (right). (a) RNA Bioanalyzer gel images (RNA 6000 Nano chips) from samples stored for 0 days. (b) RNA Bioanalyzer gel images from samples stored for 3 days. (c) RNA Bioanalyzer gel images from samples stored for 7 days. (d) RNA Bioanalyzer gel images from samples stored for 14 days. (e) RNA Bioanalyzer gel images from samples stored for 21 days. (f) DNA bioanalyzer traces from SS2 cDNA libraries generated from samples stored for 0 days. (g) DNA bioanalyzer traces from SS2 cDNA libraries generated from samples stored for 3 days. (h) DNA bioanalyzer traces (dsDNA HS chips) from SS2 cDNA libraries generated from samples stored for 7 days (i) DNA bioanalyzer traces from SS2 cDNA libraries generated from samples stored for 14 days. (j) DNA bioanalyzer traces from SS2 cDNA libraries generated from samples stored for 21 days.

FIG. 12. Smart-seq2 lysis buffer storage capacity with the addition of PVSA.

(a) Bioanalyzer traces (dsDNA HS chips) of control Smart-seq2 (SS2) cDNA libraries from HEK293FT cells FACS-sorted into lysis buffer containing no inhibitor (0 μg/mL of PVSA), 60, 90 or 120 μg/mL of PVSA, where cDNA was generated immediately after cell sorting (day 0). (b) Bioanalyzer traces of SS2 cDNA libraries from HEK293FT cells FACS-sorted into lysis buffer containing various concentrations of inhibitor processed into libraries after storage at −80° C. for 1, 4, 7, and 14 days. (c) Bioanalyzer traces of SS2 cDNA libraries from HEK293FT cells FACS-sorted into lysis buffer containing various concentrations of inhibitor after storage at 25° C. for 1, 4, 7, and 14 days (top to bottom). (d) Bioanalyzer traces of SS2 cDNA libraries from HEK293FT cells FACS-sorted into lysis buffer containing various concentrations of inhibitor after storage at 4° C. for 1, 4, 7, and 15 days (top to bottom).

FIG. 13. Library characteristics of cells stored in PVSA-containing Smart-seq3xpress lysis buffer.

(a) Box plot of number of genes detected from sequenced libraries of sorted HEK293FT cells stored in Smart-seq3xpress lysis buffer containing no inhibitor (0 μg/mL), PVSA (0.6, 1.5, 3.0, 6.0, 15, or 30 μg/mL) or RI. Day-0 data considering all sequencing reads. n=96 samples per condition. (b) Box plot of UMIs detected from sequenced libraries of sorted HEK293FT cells stored in Smart-seq3xpress lysis buffer for each inhibitor condition. Day-0 data considering all sequencing reads. (c) Box plot of fraction of UMI-containing reads detected from sequenced libraries of sorted HEK293FT cells stored in Smart-seq3xpress lysis buffer for each inhibitor condition. Day-0 data considering all sequencing reads. (d) Stacked bar plot of fraction reads from sequenced libraries mapping to intronic regions, exonic regions, intergenic regions, unmappable, or mapped to multiple genomic regions (ambiguous) for each inhibitor condition. (e) Box plot of number of genes detected for each inhibitor condition for day 0, 1, 4, 7 at 25° C. Data down sampled to 100,000 reads per cell. (f) Box plot of UMIs detected for each inhibitor condition for day 0, 1, 4, and 7 at 25° C. Data down sampled to 100,000 UMI-containing reads per cell. (g) Box plot of number of genes detected for each inhibitor condition for day 0, 1, 4, 7 and 14 at 4° C. Data down sampled to 100,000 reads per cell. (h) Box plot of UMIs detected for each inhibitor condition for day 0, 1, 4, 7 and 14 at 4° C. Data down sampled to 100,000 reads per cell.

FIG. 14. Stress test of PVSA with subsequent single-cell RNA-seq library generation.

(a) Bioanalyzer plots of Smart-seq2 (SS2) cDNA where the lysis buffer contains 30 μg/mL of PVSA. (b) Bioanalyzer plots of SS2 cDNA where lysis buffer contained 30 μg/mL of PVSA at pH 4 (left) or pH 10 (right). (c) Bioanalyzer plots of SS2 cDNA where the lysis buffer contains 30 μg/mL of a PVSA stock either in a 1 mM Tris pH 7 solution, 1 mM Tris pH 8 solution, 5 mM Tris pH 7 solution, or 5 mM Tris pH 8 solution (left to right). (d) Bioanalyzer plots of SS2 cDNA where the lysis buffer contains 30 μg/mL of a PVSA stock stored at 4° C. for 12 (left) or 24 hours (right). (e) Bioanalyzer plots of SS2 cDNA where the lysis buffer contains 30 μg/mL of a PVSA stock stored at 37° C. for 12 (left) or 24 hours (right). (f) Bioanalyzer plots of SS2 cDNA where the lysis buffer contains 30 μg/mL of a PVSA stock stored at 50° C. for 12 (left) or 24 hours (right). (g) Bioanalyzer plots of SS2 cDNA where the lysis buffer contains 30 μg/mL of a PVSA stock vortexed for 12 (left) or 24 hours (right). (h) Bioanalyzer plots of SS2 cDNA where

FIG. 15. Additional alternative inhibitor findings in SS2 cDNA library generation. (a) Bioanalyzer trace of Smart-seq2 (SS2) cDNA libraries generated from 100 pg mouse RNA with standard biological inhibitor. (b) Bioanalyzer trace of SS2 cDNA library without inhibitor. (c) Bioanalyzer traces of SS2 cDNA library with PVSA (6-120 μg/mL). (d) Bioanalyzer traces of SS2 cDNA library with heparin (0.4-40 μg/mL). (e) Bioanalyzer traces of SS2 cDNA library with sodium alginate (20-400 μg/mL). (f) Bioanalyzer traces of SS2 cDNA library with vinylsulfonic acid (VSA) (100-3000 μg/mL). (g) Bioanalyzer traces of SS2 cDNA library with dextran sulfate (0.4-10 μg/mL). (h) Bioanalyzer traces of SS2 cDNA library with fucoidan (1-40 μg/mL). (i) Bioanalyzer traces of SS2 cDNA library with 2-(N-morpholino)ethanesulfonic acid (MES) (2-16 mg/mL). (j) Box plot of percent uniquely mapped sequencing reads to the reference genome for samples from a-I. Data shown as median (belt), interquartile range (IQR) (box), and 1.5×IQR (whiskers). (k) Box plot of number of genes detected from sequencing libraries for samples from a-i. (l) Stacked barplots of genomic mapping statistics, showing fraction reads mapping to exon, intron, and intergenomic regions.

FIG. 16. PVSA compatibility with commercial RNA-seq kits.

(a) Bioanalyzer plots of final RNA sequencing libraries generated from the NEBNext RNA Sequencing Kit with 100 pg of MTTF RNA and varying amounts of PVSA (0, 2.4, or 6 μg/mL) or RI added to the lysis step. (b) Bioanalyzer plots of final RNA sequencing libraries generated from the QIAseq FX Single Cell RNA Library Kit with 100 pg of MTTF RNA and varying amounts of PVSA (0, 1.2, 3, 12, 30, or 120 μg/mL) added to the denaturation step. (c) Bioanalyzer plots of final RNA sequencing libraries generated from the TruSeq RNA Sample Preparation Kit with 100 ng of MTTF RNA and varying amounts of PVSA (0, 1.5, 15, or 150 mg/mL) added to RNA prior to RNA purification. (d) Box plot of number of genes detected for each inhibitor condition used to each kit. (e) Box plot of percent uniquely mapped sequencing reads to the reference genome for each inhibitor condition for the NEBNext (left), QiaseqFX (center), and TruSeq (right) kits. (f) Box plot of percent sequencing reads too short to be mapped to the reference genome for kit samples. (g) Box plot of the mismatch rate length for kit samples. (h) Box plot of the average insertion length for kit samples. (i) Box plot of the average deletion length for kit samples. (j) Stacked bar plot of fraction reads mapping to intronic, exonic, or intergenic regions of the mouse genome. (k) Histogram of fraction of bases along sequencing reads matching the genome, indicating read quality. (1) Histogram of fraction of bases along sequencing reads with insertion for kit samples. (m) Histogram of fraction of bases along sequencing reads with deletion for kit samples. (n) Histogram of fraction of bases along sequencing reads with substitution kit samples.

FIG. 17. Effects of chemical and biological RNase inhibitor on cDNA library yield.

(a) Bioanalyzer plots of Smart-seq2 cDNA where various amounts of PVSA were added to the lysis buffer along with the recombinant RNase inhibitor (RI), but purifying away the RT buffer components of by bead purified prior to cDNA amplification. Concentrations stated refer to PVSA in the lysis buffer. (b) Bioanalyzer plots of Smart-seq2 cDNA where standard amounts of recombinant RNase inhibitor was used in lysis and RT, while adding PVSA to the reaction before cDNA amplification by PCR. Concentrations stated refer to PVSA in the PCR reaction. (c) Bioanalyzer plots of Smart-seq2 cDNA using the standard Smart-seq2 protocol and 4 Units of RI (left), the addition of 4 Units of RI (i.e. 2×) during in lysis buffer (middle), or 2 Units of RI (the maximum additional units that could be added) during reverse transcription (left).

FIG. 18. 1H-15N HMQC of RNase A in absence and presence of PVSA. 1H-15N HMQC of RNase A in absence and presence of PVSA in 0.8×DPBS buffer pH 7.4 at 25° C. following 1 hour incubation. Residues involved in the catalytic site are indicated, as well as examples of those which are not impacted by the addition of the ligand.

FIG. 19. NMR of a hairpin RNA in absence and presence of PVSA.

Comparison of 14mer RNA in the absence and presence of PVSA in 12 mM NaP, 20 mM NaCl, 0.08 mM EDTA, pH 6.5 following incubation for 1 hour. (a) 1H-1D of H6, H2, and H8s. Minor perturbation on imino base pairing upon addition of the ligand. (b) 1H-1D of the imino region. No or minor effect on imino base pairing when the ligand is added. (c) 31P-1D with phosphate buffer suppression. No or minor chemical shift on phosphate backbone upon addition of the PVSA ligand, indicating the lack of notable interaction.

FIG. 20. Using paper PVSA-treated paper as an RNA storage medium for subsequent RNA-seq library generation.

(a) Bioanalyzer traces of Smart-seq2 (SS2) cDNA from HEK293FT cells spotted on paper strips treated with 30 mg/mL, 3 mg/mL 300 μg/mL, 30 μg/mL, 3 μg/mL, or 0 μg/mL of PVSA, eluted after 1 day with cDNA generated immediately after elution. (b) Bioanalyzer traces of SS2 cDNA from HEK293FT cells spotted on paper treated with or without PVSA, eluted after 3 days. (c) Bioanalyzer traces of SS2 cDNA from HEK293FT cells spotted on paper treated with or without PVSA, eluted after 7 days. (d) Bioanalyzer traces of SS2 cDNA from HEK293FT cells spotted on paper treated with or without PVSA, eluted after 1 day and cDNA generated 6 days later after elution was stored at 25° C. (e) Bioanalyzer trace of SS2 cDNA directly from 25 HEK293FT cells lysed in 1% Triton-X100.

EXAMPLE 1—REPLACING RECOMBINANT RNASE INHIBITOR WITH VINYLSULFONIC ACID POLYMERS IN SINGLE-CELL RNA-SEQ

Single-cell RNA-sequencing (scRNAseq) is currently revolutionizing biomedicine, propelled by advances in scRNAseq methodology, ease of use, and cost reduction of library preparation. Here, we demonstrate that recombinant RNase inhibitors used in scRNAseq can be replaced by chemical vinylsulfonic acid polymers, yielding single-cell libraries of equal or superior quality at virtually no cost for RNA inhibition. Moreover, the thermostability of the chemical RNase inhibitor enables new and simplified workflows and may increase reproducibility and throughput of RNA-seq applications.

The capture of intact RNA is a requisite of RNA-seq methods to accurately record the transcriptome of the analyzed sample material. ScRNAseq is particularly sensitive to RNA degradation and detection dropout due to the miniscule copy numbers of individual transcripts present in the cell (1,2). Thus, the inclusion of recombinant RNase inhibitors (RI), which are in vitro synthesized proteins, is customary during storage, cell-lysis, and reverse transcription (RT). However, the use of RI is inconvenient not only due to its relatively high cost fraction of the library, but also due to its degradability, which can introduce batch variation in library yield and quality due to production lot, storage time, and temperature conditions for the inhibitor.

Recent studies showed that polyvinyl sulfonic acid (PVSA) (FIG. 1a) reduced RNA cleavage activity of RNase A and ribonucleases present in Escherichia coli lysate and that it improved yield in decoupled E. coli-based cell-free protein synthesis of green fluorescent protein (GFP) (3), while it did not markedly improve detection of viral genomic RNA by RT-qPCR when supplemented into viral transport media (4). It remains completely unexplored how this compound performs as RNase inhibitor in the context of RNA-seq library generation, requiring not merely RNase inhibition of eucaryotic material but direct compatibility with transcriptases and polymerases without introducing base-pair errors or decreasing coverage, while retaining maximum sensitivity to detect transcripts.

We started by systematically testing ranges of PVSA concentrations in the scRNAseq protocol Smart-seq2 (SS2) (5), which provides full-length read coverage across transcripts, enabling evaluation of polymerase processivity. We prepared lysis buffer containing 0-600 μg/mL of PVSA (resulting in 0-270 μg/mL in the following first-strand reaction) and 100 pg spiked mouse RNA, performed SS2 library amplification, and evaluated resulting cDNA by capillary electrophoresis (Methods). Indeed, libraries generated using specific concentration ranges of PVSA were of similar size distribution and yield as standard SS2 utilizing standard RI (TaKaRa), and we identified 30-120 μg/mL PVSA as the optimal range in this assay (FIG. 1b-c). At lower and higher PVSA concentrations the cDNA yield declined due 47 to RNA degradation and polymerase inhibition, respectively (FIG. 1b-c). Intriguingly, we noticed that PVSA also increased PCR specificity, reducing primer-dimer formation in the SS2 libraries (FIG. 1a, c). We explored this property of PVSA by a dimer assay using primers intentionally designed to self-hybridize (6) and eGFP plasmids as template (Methods), indeed observing elimination of dimers but preserved amplicon formation as PVSA concentration was increased (FIG. 1e). We also designed TUBA1A-targeting primer pairs of varying tendency to form unspecific products and found that PVSA could successfully prevent unintended bands and smear products amplified from human gDNA (FIG. 1e). By melting curve analysis, we found that the PVSA-mediated stringency was not the mere result of increased melting temperature (Tm) (FIG. 3 (Supplementary FIG. 1)). Thus, PVSA may only replace RI in RNA-seq library preparation but may increase stringency and the proportion of informative fragments in the libraries.

To examine the information yield and quality of PVSA-generated RNA-seq libraries, we sequenced SS2 libraries generated in presence of PVSA or RI (TaKaRa) and downsampled the resulting reads to allow even comparison (Methods). We analyzed library quality metrics in terms of genomic and transcriptomic mappability, base substitution rates, insertion and deletions (indels), likelihood of mismatches within the read, and average indel length. PVSA conditions 100-800 μg/mL in the lysis buffer (45-360 μg/mL in RT) yielded libraries of similar quality as standard SS2 using RI with respect to all parameters investigated, including detected genes, fraction reads mapped to exons, base-along-read likelihood of matching genome, and gene body coverage (FIG. 1f-j). The lowest PSVA concentrations produced a high fraction of short and unmapped reads (FIG. 4 (Supplementary FIG. 2a, b)), as expected due to RNA degradation when excluding the inhibitor (FIG. 1a, c). The rate of substitutions and indels were indistinguishable between 30-240 μg/mL PVSA and standard SS2 (FIG. 4 (Supplementary FIG. 2c-h)).

Encouraged by these results, we evaluated PVSA in single-cell cDNA library generation. We sorted cultured human embryonic kidney cells (HEK293FT) into PCR plates containing lysis buffer and 0-480 μg/mL PVSA, or RI (Methods), and analyzed the resulting libraries. We identified a similar optimal PVSA range (60-240 μg/mL; peaking at ˜60 μg/mL) for FACS sorted single cells as previously for purified mouse RNA (FIG. 2a-c). Again, assessment of the read quality, as well as the read coverage to the transcriptome, gene detection, and per-base mappability of the reads showed that PVSA-generated libraries (60-240 μg/mL) were of equal quality as standard SS2 libraries generated using RI (FIG. 2d-f, FIG. 4 (Supplementary FIG. 2i-m)). We investigated cell-cycle signatures in PVSA-generated libraries in the yield-peak range, indeed capturing the same transcriptome signature and fraction cells in phases for PVSA- and RI-generated libraries (FIG. 5 (Supplementary FIG. 3)).

We then challenged the PVSA methodology by FACS gating for small cells dissociated from mouse spleen and liver (FIG. 6 (Supplementary FIG. 4a, b)); tissues known to be of high and moderate RNase content, respectively. Cells were sorted in lysis buffer containing either RI or PVSA (60-90 μg/mL). Importantly, library quality of the three conditions was the same (FIG. 6 (Supplementary FIG. 4d-r)). We visualized the relationship between cells and organ types by Uniform Manifold Approximation and Projection (UMAP) and observed that the distribution of the chemical and biological-inhibitor-treated samples was well-represented throughout the projection, indicating little or no difference in gene and cell-type detection between the conditions (FIG. 2g, h). We identified four distinct clusters, three which were immune cell subtypes B cell, T cell, monocytes/macrophages (clusters 0, 1, 3) and one that was enriched for liver-specific genes (cluster 2) (FIG. 2i-n), in line with liver and kidney lymphocyte/monocyte populations identified in previous studies (7, 8, 9, 10).

Having demonstrated successful replacement of RI with PVSA in SS2, we identified the optimal PVSA range in the Smartseq3 (SS3) (11) protocol using both spiked mouse RNA and sorted HEK293FT cells (FIG. 2o-r), demonstrating that 0.6-6 μg/mL PVSA in the lysis buffer (0.3-3 μg/mL in the following RT step) produced maximum cDNA yield (FIG. 2o-r) and sequencing data of parallel quality metrics as standard SS3 using RI (FIG. 2s-v and FIG. 7 (Supplementary FIG. 5)). An additional parameter provided by SS3 is the counting of original RNA molecules via unique molecular identifiers (UMIs) incorporated in the strand-switch oligo at 5′ end. Considering UMI counts only, we similarly observed that the PVSA range with maximal cDNA yield (0.6-3 μg/mL in lysis buffer) produced maximum UMI detection, on par with standard SS3 (FIG. 2t).

We explored whether PVSA could withstand prolonged storage or extended heating, remarkably finding that RNA in PVSA-containing lysis could be kept at room temperature for 24 hrs and even heated to 90° C. without apparent loss or fragmentation of RNA (FIG. 8 (Supplementary FIG. 6)). Finally, we probed the RNA protection capability of additional sulfonated, sulfated, phosphonated, and carboxylated polymers and monomers in the context of RNA-seq library generation, observing that PVPA and VPA (a phosphonated polymer and monomer) did not sustain RNA stability during cDNA library generation, while heparin (sulfated and carboxylated polymer), sodium alginate (sulfated and carboxylated polymer), dextran sulfate (sulfated polymer), sodium alginate (carboxylated and sulfated polymer), VSA (sulfonated monomer), and MES (sulfonated monomer) did (FIG. 9 (Supplementary FIG. 7)).

In summary, we report that vinylsulfonic acid polymers can replace RI in scRNAseq and bulk applications without compromising library quality. This is likely to revolutionize RNA-seq library generation. Chemical RNase inhibition has several advantages, with the obvious being that cost of RNase inhibition henceforth becomes negligible (e.g. ˜1/500,000 relative to available RIs, or ˜0.0000003 USD per SS2 library). In addition, its thermostability enables new and simplified workflows precluded by the less inert biological inhibitors. For example, stable premade sample collection buffers that can made, frozen, thawed, and kept at room-temperature for extended periods of time. For scRNAseq and multiomics applications, plates can be pre-spotted with PVSA-containing lysis buffer and need not be freshly prepared before cell collection. Furthermore, the chemical inhibitor remains effective throughout heat cycles, thus need not be supplemented twice during library preparation (i.e. lysis+RT step). This property could also be useful for in vitro RNA synthesis and structural RNA applications. Although we used a next generation based readout in the current proof-of-principle study, we anticipate the utility of chemical RNase inhibitors also in third-generation long-read sequencing of RNA and cDNA utilizing nanopores. We moreover envision that chemical RNase inhibition using polymers will be exceedingly beneficial in applications requiring large liquid volumes and high inhibitor consumption, and could facilitate in situ RNA sequencing and RNA-FISH on tissues or whole organisms, likely with increased permeability of the smaller-sized polymers.

### Methods

### Animals and Cells

C57BL/6J (B6) mice were crossed with M. castaneous (CAST/EiJ) mice to produce B6/CAST hybrid mice. Mice were housed in specific pathogen-free at Comparative Medicine Biomedicum (KM-B) according to Swedish national regulations for laboratory animal work food and water ad libitum, cage enrichment, and 12 hours light and dark cycles (ethical permit 17956-2018, Jordbruksverket). Primary fibroblasts were derived from adult CAST/EiJ×C57BL/6J mice by skinning, mincing, and culturing tail explants in fibroblast medium (DMEM/10% FBS) in 5% CO2 and 37° C. HEK293FT cells were cultured and expanded in standard medium (DMEM/10% FBS) in 5% CO2 and 37° C., and 10 million cells were collected and resuspended in 0.5% BSA in PBS for cell sorting. Mouse liver and spleen were collected from purebred C57BL/6J mice. For liver and spleen collection, 25 mL of warmed perfusion buffer (140 mM NaCl, 6.7 mM KCl, 9.6 mM HEPES, 6 mM NaOH) was slowly injected into the left ventricle after a small incision in the right ventricle. Both organs were collected on ice in perfusion buffer. The spleen was cut into pieces, mashed with a syringe plunger and filtered through a 70-um cell strainer with the addition of 2% FBS in PBS. The 201 liver was cut into 1 pieces and treated with 1 mg/mL collagenase in perfusion buffer with 3 mM CaCl2) and 1 mM MgCl2 at 37° C. with shaking for 20 minutes. Liver cells were mashed through a 70-um cell strainer with the syringe plunger, rinsing the strainer with 10% FBS in PBS. Both strained tissue types were pelleted, reconstituted in 1×RBC lysis buffer for 5 minutes at room temperature, and spun down at 500 g for 5 minutes to remove red blood cells. Spleen cells were washed with 0.5% BSA in PBS, reconstituted in 0.5% BSA in PBS, and filtered through a 40-um cell strainer. Liver cells were washed 2× with PBS, incubated with 2 mL TrypLE express for 10 minutes at 37 deg with shaking, diluted to 20 mL with 10% FBS in PBS, pelleted, reconstituted in 0.5% BSA in PBS, and filtered through a 40-um cell strainer.

### Generation of RNA-Seq Libraries

Bulk RNA was extracted from cultured mouse tail tip fibroblasts using TRIzol (Invitrogen). For all bulk RNAseq experiments 100 pg of RNA was used as input, if is not otherwise specified, and was added to 96-well plates containing either Smart-seq2 or Smart-seq3 standard lysis buffer or lysis buffer with varying concentrations of polyvinyl sulfonic acid (Merck, cat no. 278424). Smart-seq2 lysis buffer composition was 0.1% Triton X-100, 2.5 mM dNTP/each, 2.5 mM Smart seq2 oligo-dT (5′-AAGCAGTGGTATCAACGCAGAGTACT30VN-3′), and 4 U recombinant inhibitor (TaKaRa) or PVSA of the concentration specified for each condition; total buffer volume 4.5 ul. Smart-seq3 lysis buffer composition was 0.1% Triton X-100, 10% PEG 8000, 1 mM dNTP/each, and 1 uM Smart-seq3 oligo-dT (5′-biotin-ACGAGCATCAGCAGCATACGA T30VN-3′), and 1 U recombinant inhibitor (TaKaRa) or PVSA of the concentration specified for each condition; total buffer volume 2 ul. For single-cell experiments, cultured HEK293FT, spleen, or liver cells in 0.5% BSA in PBS were sorted into 96 well plates containing lysis buffer using an SH800S (Sony). Single cell sorted plates were briefly centrifuged and kept at −80° C. until they were further processed. Plates containing lysis buffer and input material were then processed according to the Smart seq2 and Smart-seq3 protocols (Picelli, S. et al., 2014 and Hagemann-Jensen, M. et al., 2020), except without addition of recombinant inhibitor in the first-strand reaction mix in reactions where PVSA was used. Smart-seq2 cDNA library generation: Cell were lysed at 72° C. for 3 min in a Bioer Life ECO thermocycler and placed on ice prior to first-strand synthesis. The following reverse transcriptase reaction contained 1× Superscript II buffer, 5 mM DTT, 1M betaine, 10 mM MgCl2, 1 uM Smart-seq2 TSO (5′-AAGCAGTGGTATCAACGCAGAGTACATrGrG+G-3′), 10 U Superscript II, and 10 U recombinant inhibitor (TaKaRa) for samples containing biological inhibitor; total reaction volume 10 ul. RT thermocycles were 42° C. for 90 min, followed by 10 cycles of 42° C. for 2 min and 50° C. for 2 min. The following cDNA amplification reaction contained 1× KAPA HiFi HotStart Ready Mix and 80 nM ISPCR primers (5′-AAGCAGTGGTATCAACGCAGAGT-3′); total reaction volume 25 ul. Thermocycles for Smart-seq2 cDNA amplification were 98° C. for 3 min, followed by either 18 (bulk), 20 (HEK), 21 (liver) or 22 (spleen) cycles of 98° C. for 20 sec, 67° C. for 15 sec, and 72° C. for 6 min, followed by a final incubation at 72° C. for 5 min. Smart-seq3 cDNA library generation: Cell were lysed at 72° C. for 10 min and placed on ice prior to first-strand synthesis. The following reverse transcriptase reaction contained 50 mM Tris-HCl (pH 8.3), 30 mM NaCl, 1 mM GTP, 8 mM DTT, 5 mM MgCl2, 2 uM Smart-seq3 TSO (5′-biotin-AGAGACAGATTGCGCAATGNNNNNNNNrGrGrG-3′), 8 U Maxima H-minus RT enzyme, and 2 U recombinant inhibitor 244 (TaKaRa) for samples containing biological inhibitor; total reaction volume 4 ul. RT thermocycles were 42° C. for 90 minutes, followed by 10 cycles of 42° C. for 2 min and 50° C. for 2 min. The following cDNA amplification reaction contained 1× KAPA HiFi buffer, 300 nM dNTP/each, 500 nM MgCl2, 500 nM forward primer (5′-TCGTCGGCAGCGTCAGATGTGTATAAGAGACAGATTGCGCAA*T*G-3′), 100 nM reverse primer (5′-ACGAGCATCAGCAGCATAC*G*A-3′), and 0.2 U KAPA polymerase in a total reaction volume of 10 ul. Thermocycles for Smart-seq3 cDNA amplification were 98° C. for 3 min, then either 20 (bulk) or 22 (single-cell) cycles of 98° C. for 20 sec, 65° C. for 30 sec, and 72° C. for 6 min, followed by a final incubation at 72° C. for 5 min. Amplified cDNA was bead purified (AMPure XP, Beckman) at a ratio of 0.8:1 beads:cDNA (20 ul beads:25 ul cDNA for Smart-seq2 and 8 ul beads:10 ul cDNA for Smart-seq3) and inspected on a Bioanalyzer 2100 (High Sensitivity DNA kit, Agilent). Purified cDNA was tagmented using custom Tn5 (Picelli S. et al., 2014). For Smart-seq2, the tagmentation reaction contained 2×TAPS-Mg buffer, 10% PEG 8000, 0.3 nM Tn5, and 1 ng of cDNA in 20 ul total, was incubated for 8 minutes at 55° C., and Tn5 was stripped from the DNA with 3.5 ul of 0.2% SDS, incubated at room temperature for 5 minutes. For Smart-seq3, the tagmentation reaction contained 10 mM Tris-HCl pH 7.5, 5 mM MgCl2, 5% dimethylformamide, and 0.08 ul (0.12 ul for single cell) Tn5 and 100 pg of cDNA in 2 ul total, was incubated for 10 minutes at 55° C., and Tn5 was stripped from the DNA with 0.5 ul of 0.2% SDS (0.4 ul of 0.2% SDS for single-cell). To amplify Smart-seq2 libraries, a reaction mix containing the tagmented cDNA, 0.75 ul of pooled dual-index primers, 1× Kapa HiFi PCR buffer, 300 uM dNTP/each, and 1 U Kapa HiFi polymerase in 50 ul total was run in a thermocycler for 3 minutes at 72° C., 95° C. 30 seconds, then 10 cycles of 95° C. for 10 seconds, 55° C. for 30 seconds, and 72° C. for 30 seconds, then 72° C. for 5 minutes. To amplify Smart-seq3 libraries, a reaction mix containing the tagmented cDNA, 0.75 ul of pooled dual index primers, 1× Phusion HiFi PCR buffer, 200 uM dNTP/each, and 0.1 U Phusion HiFi polymerase in 7 ul total was run was run in a thermocycler for 3 minutes at 72° C., 98° C. for 3 minutes, then 12 cycles of 98° C. for 10 seconds, 55° C. for 30 seconds, and 72° C. for 30 seconds, then 72° C. for 5 minutes. Indexed libraries were bead purified (AMPure XP, Beckman) at a ratio of 0.6:1 beads:cDNA (30 ul beads:50 ul library for Smart-seq2 and 4.2 ul beads:7 ul library for Smart-seq3). All Smart-seq2 libraries were sequenced as 74 bp, single-end reads, and all Smart seq2 libraries were sequenced as 74 bp, paired-end reads.

Alternative inhibitors (heparin, sodium alginate, VSA, dextran sulfate, fucoidan, MES, PVPA, and VPA) were tested following the SS2 protocol for bulk RNA. Thermostability tests were performed with the standard SS2 bulk protocol, but with a 1-hour incubation of the lysis buffer+RNA before first strand synthesis.

### Bioanalyzer DNA Yield Calculations

All bioanalyzer traces were exported in raw format as a csv file. For all histograms, the average and standard error of the area-under-curve (DNA yield) was calculated for each sample. Percent primer dimer was calculated as (primer-dimer yield)/(total library yield).

### Read Alignment and Genes/UMIs Detected

Read files were downsampled to 100k and aligned using STAR (Spliced Transcripts Alignment to a Reference) against the Gencode GRm38 (mouse) or GRCh38 (human) genome assembly. STAR aligner determined the accuracy of mapping for each sample with percent uniquely mapped reads and percent unmapped reads. STAR was also used to calculate the rate of read mismatch to the genome and length of insertions and deletions within the read relative to the genome. MultiQC's output of STAR generated a summary of all STAR QC parameters for all samples.

Data for both number of genes and UMIs detected was generated using the zUMIs pipeline (Parekh, S. et al., 2018). Briefly, downsampled loom files that contained read coverage of exons only were processed in R for gene count (readcount.exon) and UMI count (umicount.exon). The number of genes expressed for each sample was quantified as sum of every gene with >1 read fragment. The number of UMIs detected for each sample was quantified as the sum of all umi-containing fragments.

### Base Quality Along Read Length

BBMap's tool mhist was used to plot the frequency that a read's base position matched or contained a substitution/insertion/deletion relative to the genome. Output files for each type were edited to and merged as a usable matrix for downstream processing in R.

### Genomic and Gene Body Mappin

Sorted bam files generated by STAR were used in more in-depth mapping quantifications. The fraction of reads that mapped to exonic, intronic, or intergenic regions in the genome were obtained with Qualimap's RNASeQC. MultiQC's output of Qualimap generated a summary of all parameters for all samples. Gene body coverage plots were generated using computeMatrix and plotProfile via deepTools.

### Dimensionality Reduction, Cell Cycle Scoring, and Clusterin

Smart-seq2 HEK sample were downsampled to 100k reads. Gene selection and PCA-based dimensionality reduction for cell cycle genes (CellCycleScoring) were performed with Seurat. Smart-seq2 spleen and liver samples were downsampled to 250k reads and filtered to exclude cells more than 2MAD above and below the mean for genes detected and reads aligned. Variable gene selection, UMAP-based dimensionality reduction, and clustering were performed with Seurat.

### Data Visualization

All RNAseq data was plotted using ggplot2 in R. (T-tests were performed using pairwise_t_test from the package tidytests).

### PCR for Primer Dimer and Non-Specific Band Assays

The primer dimer assay was designed to produce a 164 bp amplicon from a Cas9-eGFP plasmid. The final PCR reaction mix contained 1× Hifi Kapa Mix, 1 uM of the fwd/rev primer pool, and 1 ng of plasmid. PCR reaction conditions were 95° C. for 3 min, then 25 cycles of 98° C. for 20 sec, 65° C. for 15 sec, and 72° C. for 30 sec, followed by a final extension at 72° C. for 1 min. The non-specific band assays were designed to produce amplicons between 120 and 140 bp from Tubala in the human genome. The final PCR reaction mix for the non-specific band assay contained 1× Kapa Hifi Mix, 1 uM of the fwd/rev primer pool, and 20 ng of HEK293FT gDNA. PCR thermocyles were 95° C. for 3 min, then 25 cycles of 98° C. for 20 sec, 57° C. for 15 sec, and 72° C. for 30 sec, followed by a final extension at 72° C. for 1 min.

### Melt Curve Analysis

Purified eGFP amplicons (164 bp) were used as a dsDNA template. The reaction mixture contained 1× SYBR Mix, 500 ng of template, and varying concentrations of PVSA (n=6 for each sample). The assay conditions were: 95° C. for 15 sec to denature, 40° C. for 1 min to anneal, and a temperature gradient from 40 to 95° C. at a rate for 0.1° a second. Melt curve was performed with StepOne Plus Real-Time machine (Applied Biosystems). Results were exported to csv files using the Applied Biosystems™ Analysis Software.

TABLE 1
Number of samples or replicates in experiments.

| | | | Library | Dimensionality |
|---|---|---|---|---|
| Experiment | | Bioanalyzer | QC | Reduction |
| Type | Condition | (n) | (n) | (n) |
| SS2 bulk | 0 | 7 | 5 | |
| SS2 bulk | 5 | 2 | 6 | |
| SS2 bulk | 10 | N/A | 6 | |
| SS2 bulk | 20 | N/A | 6 | |
| SS2 bulk | 50 | 2 | 6 | |
| SS2 bulk | 100 | 7 | 6 | |
| SS2 bulk | 200 | 7 | 31 | |
| SS2 bulk | 300 | N/A | 26 | |
| SS2 bulk | 400 | 7 | 35 | |
| SS2 bulk | 600 | 8 | 13 | |
| SS2 bulk | 800 | 6 | 13 | |
| SS2 bulk | 1200 | 5 | 4 | |
| SS2 bulk | 1600 | N/A | 6 | |
| SS2 bulk | 2000 | N/A | 8 | |
| SS2 bulk | SS2-RI | 3 | 24 | |
| | total | 54 | 195 | |
| SS2 scHEK293FT | 0 | 5 | 46 | N/A |
| SS2 scHEK293FT | 10 | 5 | 45 | N/A |
| SS2 scHEK293FT | 20 | 5 | 46 | N/A |
| SS2 scHEK293FT | 50 | 4 | 46 | N/A |
| SS2 scHEK293FT | 200 | 6 | 91 | 91 |
| SS2 scHEK293FT | 300 | 4 | 94 | 93 |
| SS2 scHEK293FT | 400 | 3 | 90 | N/A |
| SS2 scHEK293FT | 600 | 3 | 40 | N/A |
| SS2 scHEK293FT | 800 | 3 | 42 | N/A |
| SS2 scHEK293FT | 1200 | 4 | 37 | N/A |
| SS2 scHEK293FT | 1600 | 3 | 41 | N/A |
| SS2 scHEK293FT | SS2-RI | 5 | 86 | 86 |
| | total | 50 | 704 | 270 |
| SS2 spleen | 200 | 8 | 122 | 103 |
| SS2 spleen | 300 | 7 | 116 | 89 |
| SS2 spleen | SS2 | 7 | 119 | 88 |
| SS2 liver | 200 | 8 | 89 | 58 |
| SS2 liver | 300 | 5 | 95 | 50 |
| SS2 liver | SS2-RI | 8 | 98 | 68 |
| | total | 43 | 639 | 456 |
| SS3 bulk | 0 | 5 | 15 | |
| SS3 bulk | 0.2 | 3 | 8 | |
| SS3 bulk | 0.5 | 3 | 7 | |
| SS3 bulk | 1 | 3 | 13 | |
| SS3 bulk | 2 | 3 | 7 | |
| SS3 bulk | 5 | 4 | 15 | |
| SS3 bulk | 10 | 4 | 14 | |
| SS3 bulk | 20 | 4 | 16 | |
| SS3 bulk | 50 | 3 | 15 | |
| SS3 bulk | 75 | 3 | 15 | |
| SS3 bulk | 100 | 5 | 15 | |
| SS3 bulk | 200 | 3 | 8 | |
| SS3 bulk | 300 | 3 | 8 | |
| SS3 bulk | SS3-RI | 5 | 14 | |
| | total | 51 | 170 | |
| SS3 scHEK293FT | 5 | 14 | 45 | |
| SS3 scHEK293FT | 10 | 14 | 40 | |
| SS3 scHEK293FT | 20 | 11 | 31 | |
| SS3 scHEK293FT | SS3-RI | 14 | 42 | |
| | total | 53 | 158 | |

TABLE 2
Template and oligo sequences used in dimer assays.

| Vector | Source | | |
|---|---|---|---|
| pRP[CRISPR]-EGFP-hCas9- | https://en.vectorbuilder. | Vector builder | |
| U6 > Scramble_gRNA1 | com/vector/VB211005- | | |
| | 1089ngk.html | | |
| Primer | Sequence | Amplicon length (bp) | |
| EGFP_FW1 | AACTAGAGAACCCACTGC | 164 | |
| EGFP_RV1 | AACTTCAGGGTCAGCTTG | | |
| | | Amplicon | |
| Primer | Sequence | length (bp) | |
| hs Tubala F1 | AGCACTCTGATTGTGCCTTC | 122 | Primer pair #1 |
| hs Tubala R1 | GGACACAATTTGACCTATTAACCTATTC | | |
| hs Tubala F2 | ATGCTGAGCAACACCACA | 126 | Primer pair #3 |
| hs Tubala R2 | AAACTCACCTTCCTCCATCC | | |
| hs Tubala F6 | GGTTTCCACAGCTGTAGTTGA | 137 | Primer pair #2 |
| hs Tubala R6 | GACGCTCAATATCGAGGTTTCT | | |
| hs Tubala F7 | CGATATTGAGCGTCCAACCTAT | 120 | Primer pair #4 |
| hs Tubala R7 | GTCTGGAATTCTGTCAGGTCAA | | |
| hs Tubala F8 | TTGCCACCATCAAGACCAA | 133 | Primer pair #5 |
| hs Tubala R8 | CACACAGCTCTCTGTACCTTG | | |

TABLE 3
Variable genes identified in liver and spleen cells
and Z-score of gene expression in cell clusters.

| Gene | Cluster 0 | Cluster 1 | Cluster 2 | Cluster 3 |
|---|---|---|---|---|
| Lyz2 | −0.2374 | −0.0894 | −0.1177 | 3.3049 |
| Gzma | −0.2392 | 0.7422 | 0.0401 | −0.1302 |
| Iglv1 | 0.1275 | −0.2845 | −0.0545 | −0.2898 |
| Igkv19-93 | 0.0964 | −0.2114 | −0.0486 | −0.2196 |
| Igkv9-120 | 0.0578 | −0.1528 | 0.0689 | −0.2172 |
| Igkv6-15 | 0.0718 | −0.1456 | −0.0377 | −0.206 |
| Gzmb | −0.2177 | 0.7145 | −0.0028 | −0.1931 |
| Ccl5 | −0.3303 | 1.1679 | −0.1885 | −0.2658 |
| Igkv8-24 | 0.0653 | −0.1519 | 0 | −0.1772 |
| Iglc1 | 0.2591 | −0.5649 | −0.1591 | −0.5497 |
| Igkv1-117 | 0.146 | −0.3306 | −0.1582 | −0.1339 |
| Ifitm3 | −0.2151 | −0.1673 | −0.1751 | 3.4522 |
| Igkv12-44 | 0.0861 | −0.1835 | −0.0485 | −0.207 |
| Igkv1-135 | 0.1333 | −0.2631 | −0.1566 | −0.2471 |
| Lyz1 | −0.2292 | −0.1428 | −0.1593 | 3.492 |
| Igkv17-121 | 0.1029 | −0.1798 | −0.1571 | −0.2104 |
| Nr4a1 | −0.165 | −0.1525 | 0.2852 | 1.9511 |
| Alb | −0.4186 | 0.2518 | 2.0198 | 0.0854 |
| Igkv10-96 | 0.0742 | −0.155 | −0.019 | −0.2333 |
| Cd8b1 | −0.1511 | 0.6066 | −0.1814 | −0.2168 |
| mt-Cytb | −0.1887 | 0.0709 | 1.2064 | −0.3563 |
| Cxcr6 | −0.2867 | 1.0208 | −0.1759 | −0.2342 |
| Ighv9-3 | 0.0296 | −0.1521 | 0.2166 | −0.1716 |
| Iglv2 | 0.0883 | −0.2319 | 0.0806 | −0.291 |
| Nkg7 | −0.3996 | 1.3898 | −0.1981 | −0.2893 |
| Tyrobp | −0.259 | 0.2291 | −0.0625 | 2.2411 |
| Cd7 | −0.2634 | 0.9082 | −0.1133 | −0.1934 |
| Igkv8-30 | 0.0401 | −0.1411 | 0.1066 | −0.1318 |
| Apoe | −0.3449 | −0.0224 | 1.5147 | 1.2249 |
| Ighv1-64 | 0.0804 | −0.1788 | −0.0354 | −0.183 |
| Dnase1l3 | 0.1269 | −0.3482 | −0.002 | −0.1404 |
| AW112010 | −0.3743 | 1.3534 | −0.3275 | −0.1998 |
| Igkv1-110 | 0.149 | −0.2994 | −0.222 | −0.167 |
| Ccl4 | −0.1706 | 0.4996 | −0.1106 | 0.282 |
| Prf1 | −0.2012 | 0.7038 | −0.1045 | −0.1522 |
| Siglech | −0.0201 | −0.0912 | 0.1035 | 0.3081 |
| Ighv1-75 | 0.0956 | −0.1526 | −0.1823 | −0.1823 |
| Igkv3-2 | 0.0856 | −0.1385 | −0.1848 | −0.1163 |
| Ly6c2 | −0.2443 | 0.4047 | −0.224 | 1.7076 |
| Psap | −0.039 | −0.2499 | −0.1576 | 1.6965 |
| Ncr1 | −0.2172 | 0.7183 | −0.0544 | −0.116 |
| Ighv1-76 | 0.0447 | −0.1103 | 0.0258 | −0.1459 |
| Igkv3-4 | 0.0203 | −0.1351 | 0.0628 | 0.1609 |
| Mpeg1 | −0.1339 | −0.2622 | −0.084 | 2.7015 |
| Igkv8-27 | 0.0705 | −0.085 | −0.1749 | −0.1749 |
| Igkv6-32 | 0.0319 | −0.0891 | −0.1324 | 0.205 |
| Igkv16-104 | 0.0206 | −0.0885 | 0.0775 | −0.0471 |
| Ccl9 | −0.1877 | −0.1725 | −0.2331 | 3.264 |
| Igkv13-85 | 0.0549 | −0.137 | 0.0166 | −0.147 |
| Igkv14-111 | 0.0744 | −0.1444 | −0.1515 | −0.0262 |
| Ctsw | −0.3809 | 1.3259 | −0.1502 | −0.3523 |
| Gm10800 | −0.1527 | 0.1115 | 0.6844 | 0.0546 |
| Ighv1-9 | 0.0793 | −0.1268 | −0.151 | −0.151 |
| Igkv17-127 | 0.1301 | −0.2107 | −0.2423 | −0.2481 |
| Lat | −0.3473 | 1.2918 | −0.2794 | −0.3688 |
| Igkv6-17 | −0.0035 | −0.1262 | 0.1907 | 0.1619 |
| Igkv4-59 | 0.1178 | −0.2041 | −0.1936 | −0.2221 |
| Fn1 | −0.2316 | −0.0771 | 0.111 | 2.761 |
| Fcer1g | −0.3336 | 0.4002 | −0.0788 | 2.4835 |
| Igkv6-23 | 0.0264 | −0.0887 | 0.0964 | −0.149 |
| Igkv12-46 | 0.0098 | −0.1477 | 0.3197 | −0.1596 |
| Klrb1c | −0.2892 | 0.9846 | −0.1375 | −0.1405 |
| Sat1 | −0.0698 | −0.1452 | −0.0901 | 1.5279 |
| Chil3 | −0.1591 | −0.0891 | −0.1616 | 2.4819 |
| Csf1r | −0.2216 | −0.1577 | 0.0097 | 3.1423 |
| Traf1 | −0.136 | 0.5802 | −0.1746 | −0.3041 |
| Hba-a1 | 0.1178 | −0.1942 | −0.2151 | −0.2192 |
| Trbc2 | −0.4553 | 1.6382 | −0.2801 | −0.4347 |
| Igkv5-48 | 0.1101 | −0.1921 | −0.1811 | −0.2024 |
| Klra7 | −0.1828 | 0.6488 | −0.1291 | −0.1097 |
| Gm43218 | 0.0443 | −0.1304 | −0.0007 | −0.0792 |
| Fah | −0.0012 | −0.0307 | 0.1397 | −0.1382 |
| Igkv5-43 | 0.0604 | −0.1617 | 0.0409 | −0.1606 |
| Ighv14-2 | 0.006 | −0.1413 | 0.3237 | −0.1413 |
| Aldob | −0.1083 | −0.0685 | 0.6983 | 0.0642 |
| Ighv1-59 | 0.0408 | −0.2144 | 0.2363 | −0.1012 |
| Ighv1-39 | 0.0272 | −0.12 | 0.1065 | −0.12 |
| Lgals1 | −0.3154 | 0.8843 | −0.1705 | 0.6062 |
| Anxa2 | −0.3142 | 0.558 | −0.2118 | 1.9096 |
| Zfp688 | 0.0162 | −0.0177 | 0.0626 | −0.2375 |
| Zbtb32 | 0.0233 | −0.0555 | −0.1252 | 0.1519 |
| Klrk1 | −0.3003 | 1.0308 | −0.1051 | −0.2475 |
| Gm10801 | −0.0766 | −0.0078 | 0.5006 | −0.051 |
| mt-Nd1 | −0.1411 | −0.0472 | 1.1268 | −0.3076 |
| Ccr9 | −0.0676 | 0.1916 | −0.0789 | 0.2018 |
| Ighv8-12 | 0.0461 | −0.1382 | 0.0582 | −0.1316 |
| Cma1 | −0.1319 | 0.4975 | −0.1137 | −0.1525 |
| Kctd2 | 0.0577 | −0.062 | −0.143 | −0.162 |
| Ighv1-22 | 0.0404 | −0.1374 | 0.0601 | −0.1374 |
| Ighv1-81 | 0.0262 | −0.1148 | 0.1564 | −0.1605 |
| Lgals3 | −0.202 | −0.0139 | −0.2355 | 2.8305 |
| Klra13-ps | −0.1664 | 0.6507 | −0.1878 | −0.1958 |
| Cd160 | −0.2946 | 1.0365 | −0.1642 | −0.2249 |
| Hbb-bs | 0.1232 | −0.216 | −0.2122 | −0.216 |
| Il2rb | −0.4221 | 1.4102 | −0.1168 | −0.2595 |
| Hpn | −0.0951 | 0.0286 | 0.5001 | 0.0266 |
| Apoa1 | −0.3873 | 0.2281 | 1.9828 | −0.118 |
| Igkv6-20 | 0.0456 | −0.1073 | −0.1073 | −0.1073 |
| Serpina3k | −0.3294 | 0.0751 | 1.8744 | −0.0017 |
| Ighv3-6 | 0.0982 | −0.2058 | −0.0629 | −0.2355 |
| Hp | −0.2632 | −0.1383 | 0.8269 | 2.013 |
| Igkv4-80 | 0.0107 | −0.0976 | 0.0605 | −0.1026 |
| Ighv2-2 | 0.0616 | −0.0936 | −0.131 | −0.131 |
| Cd3g | −0.4338 | 1.55 | −0.2234 | −0.4554 |
| Ahsg | −0.367 | 0.1705 | 1.9023 | 0.0187 |
| Plbd1 | −0.1221 | −0.2697 | −0.0799 | 2.5865 |
| Tppp3 | −0.1302 | −0.119 | −0.1425 | 2.207 |
| Cd68 | −0.1982 | −0.1686 | −0.0532 | 3.0314 |
| Slc35a4 | −0.0424 | 0.1815 | −0.0876 | −0.0353 |
| Ighv10-1 | 0.0525 | −0.1471 | 0.0601 | −0.1612 |
| Stmn1 | −0.0139 | 0.1177 | −0.0649 | −0.1641 |
| Ifit3 | −0.0275 | 0.0141 | −0.2009 | 0.6418 |
| Trbv13-3 | −0.0937 | 0.3708 | −0.1188 | −0.1188 |
| Ighv5-17 | 0.0337 | −0.1152 | 0.0585 | −0.09 |
| Cd8a | −0.1745 | 0.5874 | −0.1331 | 0.0349 |
| Ighv3-1 | 0.0727 | −0.1199 | −0.1409 | −0.1533 |
| Igkv4-57-1 | 0.03 | −0.0871 | −0.0912 | −0.0985 |
| Tcf19 | −0.0895 | 0.3011 | −0.09 | −0.1039 |
| Ighv1-80 | 0.0854 | −0.1445 | −0.1437 | −0.1682 |
| Fgb | −0.322 | −0.053 | 2.0951 | −0.0157 |
| Ighv6-3 | 0.0583 | −0.1134 | −0.0366 | −0.1741 |
| Tmem176b | −0.1807 | 0.0793 | 0.0817 | 1.6332 |
| Krtcap3 | 0.004 | 0.008 | −0.0889 | 0.0717 |
| Gm10717 | −0.1082 | 0.1026 | 0.3989 | 0.0137 |
| Rgs2 | −0.0557 | −0.1784 | −0.1863 | 1.6715 |
| Igkv3-12 | 0.0008 | −0.0694 | 0.1158 | −0.1238 |
| Ighv1-55 | 0.0161 | −0.1776 | 0.2087 | 0.0961 |
| Tgfbi | −0.1834 | −0.1491 | −0.1747 | 3.0158 |
| Car2 | −0.0937 | 0.2485 | 0.192 | −0.2221 |
| Nudt16 | −0.0592 | −0.0194 | 0.3387 | 0.1218 |
| Lag3 | −0.0638 | 0.1358 | 0.063 | 0.1037 |
| Tmem201 | −0.0181 | 0.1633 | −0.125 | −0.1758 |
| Ighv8-8 | 0.0438 | −0.1731 | 0.2069 | −0.237 |
| Ift88 | 0.0408 | −0.0995 | −0.0254 | −0.1374 |
| Gpr137 | 0.0186 | −0.0648 | 0.1064 | −0.1681 |
| Gm21738 | −0.1162 | 0.0724 | 0.4701 | 0.0788 |
| Cd5 | −0.2305 | 0.8744 | −0.2353 | −0.216 |
| Igkv12-98 | 0.0485 | −0.1057 | −0.1126 | −0.1126 |
| Trbv1 | −0.1085 | 0.2566 | 0.1109 | 0.0227 |
| Ccrl2 | −0.0788 | −0.0787 | −0.1067 | 1.3421 |
| Ifi44 | −0.0546 | 0.1074 | −0.1053 | 0.3081 |
| Stra6l | −0.0246 | −0.1249 | 0.2218 | 0.249 |
| Hba-a2 | 0.1052 | −0.1805 | −0.1868 | −0.2082 |
| Rab3d | −0.0891 | 0.1626 | −0.0646 | 0.4511 |
| Klrg1 | −0.1159 | 0.3885 | −0.0043 | −0.1287 |
| Nsun5 | 0.0338 | −0.054 | −0.1576 | 0.1105 |
| Ifit1 | −0.0787 | 0.1363 | −0.1708 | 0.7128 |
| Rgs1 | −0.1993 | 0.7528 | −0.1761 | −0.2253 |
| Saraf | −0.2758 | 0.9831 | −0.2486 | −0.0804 |
| Grn | −0.11 | −0.2455 | 0.2032 | 1.8226 |
| Igkv5-39 | −0.0275 | −0.0785 | 0.3767 | −0.1039 |
| Ccl3 | −0.218 | 0.58 | −0.1986 | 0.6896 |
| Serpina1a | −0.3653 | 0.1093 | 2.0317 | −0.012 |
| Tcn2 | −0.1018 | 0.0507 | 0.2379 | 0.5365 |
| Clu | −0.1394 | −0.006 | 0.8899 | −0.0465 |
| Ighv1-15 | 0.0129 | 0.0207 | −0.1142 | −0.1142 |
| Cfp | 0.0654 | −0.2998 | −0.1948 | 0.7489 |
| Avpi1 | 0.0351 | −0.1193 | −0.1074 | 0.1291 |
| Pofut1 | 0.0234 | 0.0062 | −0.0202 | −0.2566 |
| Rpusd1 | 0.0028 | 0.032 | 0.0008 | −0.1555 |
| Igkv4-55 | 0.0727 | −0.1347 | −0.1512 | −0.1512 |
| mt-Rnr1 | −0.3444 | 0.2409 | 1.7906 | −0.3009 |
| Rtn4ip1 | 0.0625 | −0.1253 | −0.0505 | −0.1517 |
| Mki67 | −0.0225 | 0.0159 | 0.1166 | −0.0203 |
| Klra4 | −0.1145 | 0.3902 | −0.0667 | −0.0695 |
| Gpx3 | −0.0818 | −0.028 | 0.3074 | 0.3132 |
| Ghdc | 0.025 | −0.028 | −0.1292 | 0.0607 |
| Vpreb3 | 0.2179 | −0.5052 | −0.1949 | −0.2331 |
| Ighv6-6 | 0.0376 | −0.1006 | −0.1006 | −0.1006 |
| Prickle3 | −0.0213 | 0.0055 | −0.0099 | 0.2437 |
| Ighv5-4 | 0.069 | −0.1188 | −0.1334 | −0.1421 |
| Kti12 | 0.04 | −0.1196 | 0.1152 | −0.2246 |
| Ighv1-26 | 0.0996 | −0.2365 | −0.1521 | 0.0328 |
| Izumo1r | −0.1029 | 0.4713 | −0.2098 | −0.2073 |
| Serpinale | −0.3343 | 0.1329 | 1.8524 | −0.1227 |
| mt-Nd4 | −0.2601 | 0.1349 | 1.4304 | −0.1949 |
| Aldoc | −0.0128 | −0.0675 | 0.1246 | −0.0901 |
| Dnajb12 | 0.0476 | −0.0893 | −0.0514 | −0.1424 |
| Ighv1-50 | 0.0068 | −0.1095 | 0.1336 | −0.1235 |
| Itgb2 | −0.4008 | 1.0229 | −0.1702 | 1.0667 |
| Zfp938 | 0.0231 | −0.0483 | −0.0144 | −0.1083 |
| Id2 | −0.3734 | 1.2645 | −0.2066 | −0.1002 |
| Ccr2 | −0.2874 | 0.3837 | −0.2002 | 2.2404 |
| Cln6 | −0.1022 | 0.0627 | −0.0889 | 0.9135 |
| Cd300a | −0.2204 | −0.182 | −0.0415 | 3.3168 |
| Psen1 | −0.0326 | 0.168 | −0.1535 | 0.0274 |
| Ugt3a2 | −0.1143 | −0.1089 | 0.7832 | 0.0931 |
| Vmac | 0.0449 | −0.1041 | 0.0362 | −0.1919 |
| Ubxn8 | 0.0008 | −0.0633 | −0.1031 | 0.4247 |
| Sema4a | −0.2732 | 0.8189 | −0.1941 | 0.4111 |
| Zscan21 | 0.0214 | −0.0129 | −0.1195 | −0.1195 |
| Edaradd | 0.1414 | −0.2701 | −0.1049 | −0.4112 |
| Glyat | −0.0851 | −0.0887 | 0.5553 | 0.0166 |
| Cd320 | 0.0168 | 0.0154 | −0.0895 | −0.0842 |
| Ighv1-82 | 0.0259 | −0.1261 | 0.0433 | 0.0979 |
| P2ry14 | −0.0611 | −0.0958 | 0.0748 | 0.9267 |
| Serpina3g | 0.0495 | 0.0217 | −0.2605 | −0.165 |
| Serpina3f | −0.1004 | 0.2882 | −0.0841 | −0.0019 |
| Ighv2-5 | 0.0474 | −0.1226 | −0.0277 | −0.1386 |
| Msh5 | 0.0664 | −0.1587 | −0.0115 | −0.1587 |
| Igkv14-100 | 0.0464 | −0.099 | −0.1064 | −0.081 |
| Alas1 | −0.041 | 0.0577 | 0.112 | 0.0439 |
| Trbv12-1 | −0.0956 | 0.3575 | −0.1232 | −0.0966 |
| Igkv6-29 | 0.0546 | −0.1063 | −0.1148 | −0.1148 |
| Igkv13-84 | 0.0392 | −0.1256 | 0.0556 | −0.1498 |
| Ighv1-19 | −0.0148 | −0.1042 | 0.3133 | −0.1282 |
| Trbc1 | −0.4014 | 1.4852 | −0.3151 | −0.4115 |
| Hbb-bt | 0.1131 | −0.201 | −0.201 | −0.201 |
| Ifrd2 | 0.0322 | 0.0203 | −0.1565 | −0.1565 |
| Fiz1 | 0.0092 | −0.0918 | 0.0005 | 0.2415 |
| Tmem176a | −0.0997 | −0.0021 | 0.2232 | 0.7402 |
| Ms4a4b | −0.4382 | 1.5788 | −0.3414 | −0.2915 |
| Clec7a | −0.1693 | −0.1333 | −0.1454 | 2.7372 |
| Egr1 | −0.0092 | −0.0286 | 0.2297 | −0.2171 |
| Lck | −0.3953 | 1.4456 | −0.2647 | −0.4263 |
| Gm45804 | 0.015 | −0.0939 | 0.0036 | 0.0661 |
| Lynx1 | 0.1306 | −0.2933 | −0.2598 | 0.093 |
| Gstk1 | 0.0079 | −0.0708 | 0.1032 | −0.0763 |
| Igkv8-16 | 0.0393 | −0.1146 | 0.0266 | −0.0874 |
| Pdcd1 | −0.0966 | 0.3203 | −0.0966 | −0.0966 |
| Fcgr4 | −0.1144 | −0.1016 | 0.1913 | 1.2417 |
| Gm9733 | −0.1637 | −0.1227 | −0.1818 | 2.7005 |
| Igkv1-122 | 0.0311 | −0.0889 | −0.092 | −0.0987 |
| Selenbp1 | −0.0554 | 0.0104 | 0.2999 | −0.0497 |
| Fam129c | 0.194 | −0.4199 | −0.2494 | −0.1785 |
| Trbv3 | −0.1295 | 0.4575 | −0.0665 | −0.1165 |
| Pccb | 0.0315 | −0.1427 | 0.0675 | 0.0514 |
| Bst2 | −0.1474 | 0.1392 | 0.0267 | 1.1244 |
| Klra8 | −0.1243 | 0.4432 | −0.1243 | −0.1243 |
| Ighv14-4 | 0.072 | −0.1564 | −0.0587 | −0.1698 |
| Tuba4a | 0.0168 | −0.0121 | −0.0502 | −0.0539 |
| Hagh | 0.0119 | −0.0676 | −0.0254 | 0.1666 |
| Otub2 | 0.0162 | −0.1296 | −0.0681 | 0.3531 |
| Ms4a6c | −0.213 | −0.1097 | −0.172 | 3.2025 |
| Fdxr | 0.0438 | −0.0962 | 0.0129 | −0.1652 |
| Khk | −0.0928 | −0.1053 | 0.3842 | 0.751 |
| Slc52a2 | −0.0342 | 0.1171 | 0.0072 | −0.0631 |
| Gatm | −0.0825 | −0.0291 | 0.3282 | 0.2532 |
| Plscr1 | −0.0577 | 0.1854 | 0.0299 | −0.1499 |
| Zfp563 | −0.0118 | 0.0776 | −0.0435 | −0.14 |
| Plac8 | 0.1219 | −0.5084 | −0.412 | 1.2978 |
| Trim68 | 0.0408 | −0.0392 | −0.1822 | −0.0239 |
| Ifit3b | −0.0078 | 0.0074 | −0.2436 | 0.5203 |
| Bzw2 | −0.0538 | 0.3074 | −0.2112 | −0.1496 |
| Dok2 | −0.3495 | 1.1543 | −0.1273 | −0.1072 |
| Serping1 | −0.1541 | −0.0348 | 0.9448 | 0.0728 |
| Pdgfb | −0.1108 | 0.2675 | −0.0972 | 0.4465 |
| Fpgs | −0.0295 | −0.0025 | 0.1184 | 0.1273 |
| Cd247 | −0.3093 | 1.0759 | −0.1154 | −0.2955 |
| Tcf7 | −0.2502 | 0.8952 | −0.1459 | −0.2347 |
| Nmnat3 | 0.0032 | 0.1058 | −0.1688 | −0.1557 |
| Dand5 | −0.0363 | 0.0773 | −0.0716 | 0.2039 |
| Cd82 | −0.1494 | 0.4775 | −0.0812 | 0.0646 |
| Samd10 | 0.0199 | −0.0157 | −0.0248 | −0.1736 |
| Klra5 | −0.1039 | 0.3465 | −0.0575 | −0.1204 |
| Gemin6 | 0.0559 | −0.0396 | −0.187 | −0.1437 |
| Gm43336 | 0.0093 | 0.0227 | −0.1256 | −0.1256 |
| Gm10125 | −0.0219 | 0.0143 | 0.112 | −0.103 |
| Shkbp1 | 0.0438 | −0.1021 | −0.0858 | 0.0432 |
| Ttr | −0.2978 | 0.0015 | 1.7881 | 0.0748 |
| Ftsj1 | 0.0427 | 0.0098 | −0.2101 | −0.1353 |
| F13a1 | −0.1626 | −0.1369 | −0.1733 | 2.7253 |
| Mcm2 | −0.0581 | 0.1951 | −0.0175 | −0.0371 |
| Reep4 | −0.0254 | 0.1081 | −0.0098 | −0.0987 |
| Surf1 | −0.0221 | 0.0058 | 0.1373 | −0.0245 |
| Tlcd2 | −0.0042 | −0.0895 | −0.0895 | 0.3093 |
| Syne1 | −0.0463 | 0.1273 | 0.0906 | −0.1191 |
| Alg6 | 0.0443 | −0.0434 | −0.1422 | −0.1422 |
| Igkv4-70 | 0.0946 | −0.1637 | −0.1637 | −0.1637 |
| Slc48a1 | 0.0071 | −0.0068 | −0.0598 | 0.056 |
| N4bp3 | 0.0451 | −0.0941 | −0.141 | 0.0803 |
| Fastkd2 | 0.0647 | −0.1406 | −0.0201 | −0.1762 |
| Zfp623 | −0.0548 | 0.1547 | 0.0755 | −0.1064 |
| Serpina1b | −0.2754 | −0.0168 | 1.8022 | −0.1408 |
| Irak1 | 0.0162 | −0.1031 | −0.0042 | 0.2121 |
| Nucb1 | −0.0702 | 0.2838 | −0.1768 | 0.0653 |
| Rab1b | −0.0397 | 0.1968 | −0.3096 | 0.293 |
| Rapgef3 | 0.0002 | −0.1117 | 0.1349 | 0.0357 |
| Dyrk1b | −0.0095 | 0.0732 | −0.036 | −0.1586 |
| Ttc30a1 | 0.0296 | −0.0954 | −0.0954 | −0.0546 |
| Usp18 | −0.0223 | −0.0195 | −0.1619 | 0.6365 |
| Cd177 | −0.1397 | −0.1114 | −0.0923 | 2.1878 |
| Pskh1 | 0.039 | −0.1243 | 0.132 | −0.2261 |
| Coq8a | 0.0289 | −0.2756 | 0.5614 | −0.342 |
| Enc1 | −0.0934 | 0.309 | −0.0025 | −0.1418 |
| Pcbd1 | −0.0916 | −0.096 | 0.5849 | 0.1144 |
| Lin37 | 0.0009 | −0.0008 | 0.055 | −0.1113 |
| Spp2 | −0.1203 | −0.062 | 0.6898 | 0.1029 |
| Tubg1 | 0.0323 | −0.0085 | −0.1465 | −0.0658 |
| Kxd1 | −0.0419 | −0.0105 | −0.0676 | 0.6518 |
| Sdf2l1 | 0.0131 | −0.036 | −0.1072 | 0.187 |
| Tubd1 | 0.0157 | −0.0638 | −0.1077 | 0.2635 |
| Uap1l1 | 0.0442 | −0.0799 | −0.2215 | 0.209 |
| Gm10719 | −0.0433 | −0.0519 | 0.2615 | −0.0163 |
| Oxnad1 | −0.0097 | −0.0147 | 0.0808 | −0.1264 |
| Gm37706 | 0.0634 | −0.1544 | −0.0145 | −0.1723 |
| Gm26870 | −0.0742 | −0.0074 | 0.3479 | 0.0441 |
| Cxcr3 | −0.3489 | 1.1529 | −0.0333 | −0.2847 |
| Dph2 | 0.0119 | 0.0868 | −0.1877 | −0.1148 |
| Igha | 0.058 | −0.119 | −0.1076 | −0.0991 |
| Smim5 | −0.0286 | −0.0509 | −0.0167 | 0.5408 |
| Pacsin1 | −0.0137 | 0.1119 | −0.1266 | −0.0294 |
| Ms4a6d | −0.1607 | −0.0452 | −0.0513 | 2.1257 |
| Sh2b1 | 0.0109 | 0.0026 | −0.0745 | 0.0035 |
| Ppp2ca | 0.0203 | −0.028 | −0.0025 | −0.1241 |
| Spsb2 | 0.0443 | −0.1178 | −0.1272 | 0.1744 |
| Slc35b2 | −0.0347 | 0.0561 | −0.073 | 0.3258 |
| Phactr2 | −0.0527 | −0.0412 | −0.0169 | 0.7977 |
| Slc15a3 | −0.013 | −0.0077 | −0.1602 | 0.4658 |
| Al182371 | −0.0548 | −0.1 | 0.316 | 0.2342 |
| F2 | −0.1473 | 0.0275 | 0.9557 | −0.1986 |
| Eaf2 | 0.043 | −0.1011 | −0.0335 | −0.1168 |
| Kcnb1 | 0.0884 | −0.1586 | −0.1629 | −0.1367 |
| Spg20 | −0.0346 | 0.0811 | −0.041 | 0.1469 |
| Tsc22d1 | 0.0344 | −0.1756 | −0.0061 | 0.2814 |
| Atf3 | −0.1701 | −0.1483 | −0.0489 | 2.6218 |
| Sit1 | −0.1095 | 0.4177 | −0.0335 | −0.2585 |
| Hmgxb4 | 0.0135 | −0.0246 | −0.0853 | 0.098 |
| Kif23 | −0.0593 | 0.1637 | −0.1084 | 0.0551 |
| AW209491 | −0.0438 | −0.0085 | 0.3042 | −0.1149 |
| Gm16001 | 0.0149 | −0.0058 | −0.1123 | −0.1123 |
| Klri2 | −0.1608 | 0.593 | −0.1582 | −0.0972 |
| Fmo1 | −0.0868 | −0.0868 | 0.557 | 0.0263 |
| Nop2 | −0.0492 | 0.1834 | −0.1198 | 0.0966 |
| Plin2 | −0.1958 | −0.1011 | 0.1143 | 2.4327 |
| Cst3 | −0.1055 | −0.1593 | −0.1283 | 2.0659 |
| Ssbp4 | −0.1 | 0.3421 | −0.1988 | 0.2297 |
| Wdr18 | 0.0775 | −0.1211 | −0.1235 | −0.2037 |
| Dolk | −0.1094 | 0.1919 | 0.3793 | −0.178 |
| Hfe | −0.1439 | −0.0846 | −0.1685 | 2.3021 |
| Ly6a2 | −0.1786 | −0.0354 | −0.1842 | 2.5454 |
| Plekhm2 | −0.0257 | 0.1347 | −0.1393 | −0.0416 |
| Rab31 | −0.0368 | −0.1275 | −0.2377 | 1.3562 |
| Rps6kb2 | −0.0134 | 0.0877 | −0.1522 | 0.1078 |
| Gm34220 | 0.0167 | −0.0511 | −0.1237 | 0.0205 |
| C5ar1 | −0.1356 | −0.0754 | −0.1235 | 2.0687 |
| Egr2 | −0.0006 | −0.0872 | 0.2519 | −0.1487 |
| Tha1 | −0.1054 | 0.3761 | −0.0404 | −0.1674 |
| Trp53i13 | 0.0406 | −0.1133 | 0.0797 | −0.1885 |
| R3hcc1 | −0.0313 | 0.2148 | −0.1365 | −0.1982 |
| Sfxn1 | 0.0107 | 0.0472 | −0.0523 | −0.2044 |
| Ctsb | −0.1573 | 0.0462 | −0.1029 | 1.8359 |
| Atp9a | −0.014 | 0.0759 | −0.103 | −0.103 |
| Sh3pxd2a | 0.104 | −0.248 | −0.1059 | −0.0614 |
| Pdcd7 | 0.0344 | −0.0422 | −0.1087 | −0.0688 |
| Gna12 | 0.0227 | −0.1267 | 0.0131 | 0.0559 |
| Alg3 | 0.0373 | 0.0148 | −0.2253 | −0.0638 |
| Dhx58 | −0.0127 | −0.0842 | −0.0749 | 0.6069 |
| Pla2g7 | −0.181 | −0.1423 | −0.1622 | 2.9384 |
| Tnfrsf25 | −0.1321 | 0.4486 | −0.0644 | −0.0879 |
| Adck5 | 0.0221 | 0.0508 | −0.1798 | −0.1102 |
| Fbxo34 | 0.0289 | −0.1053 | −0.1237 | 0.116 |
| Osbpl5 | −0.054 | 0.1021 | 0.1285 | −0.005 |
| Slc7a7 | −0.0204 | −0.1281 | −0.0052 | 0.6285 |
| Idh1 | −0.1213 | −0.151 | 0.309 | 1.3951 |
| Acad10 | 0.027 | −0.1061 | −0.0092 | −0.0509 |
| Tnfrsf9 | −0.1173 | 0.3012 | −0.0891 | 0.1941 |
| Haus4 | 0.0546 | −0.0804 | −0.1886 | 0.0285 |
| Igkv4-91 | 0.0032 | −0.1084 | 0.1131 | −0.0449 |
| Kyat1 | 0.0749 | −0.1086 | −0.1346 | −0.2005 |
| Slc39a3 | 0.0054 | 0.0002 | 0.0792 | −0.2118 |
| Dnase1l2 | −0.0802 | 0.0233 | 0.3462 | −0.0829 |
| Cyb5rl | −0.054 | −0.0827 | 0.0451 | 0.5947 |
| Rnf215 | −0.001 | 0.0034 | −0.1216 | 0.2267 |
| Pomt1 | 0.0458 | −0.0947 | −0.1075 | 0.0329 |
| Ighv5-16 | 0.0473 | −0.1161 | −0.0859 | −0.1161 |
| Trbv19 | −0.1128 | 0.3439 | 0.0333 | −0.1312 |
| Ccnd2 | −0.2372 | 1.0141 | −0.3789 | −0.3989 |
| Ighv2-6 | −0.005 | −0.1025 | 0.151 | −0.1114 |
| Rad51ap1 | 0.0323 | −0.1176 | −0.0514 | −0.1176 |
| Riox1 | 0.0025 | 0.0111 | −0.0952 | −0.0952 |
| Aldh1b1 | −0.0438 | 0.04 | −0.1556 | 0.6411 |
| Colq | −0.065 | 0.1556 | 0.1308 | −0.0919 |
| Thbs1 | −0.1234 | −0.0557 | −0.0935 | 1.7748 |
| Fam214b | −0.009 | −0.1569 | −0.0799 | 0.8509 |
| Ap1m2 | 0.0025 | −0.0018 | −0.1064 | −0.0563 |
| Atp13a2 | 0.064 | −0.2062 | −0.1321 | 0.2923 |
| Cd63 | −0.0453 | −0.1026 | 0.3326 | 0.0343 |
| Fam3a | −0.0453 | 0.0815 | 0.1541 | −0.0761 |
| Ndc1 | 0.0596 | −0.1458 | 0.0381 | −0.2058 |
| Ube2l6 | −0.0113 | −0.1306 | −0.0375 | 0.698 |
| Sord | −0.1068 | −0.0623 | 0.519 | 0.3109 |
| Ms4a6b | −0.3971 | 1.1411 | −0.2279 | 0.6825 |
| Ramp1 | −0.1896 | 0.4003 | −0.1841 | 1.0164 |
| Spata2l | −0.0512 | 0.0237 | 0.1673 | 0.1556 |
| Pqlc2 | 0.0204 | 0.0518 | −0.1876 | −0.0797 |
| Gm28437 | −0.1633 | −0.0327 | 1.2112 | −0.265 |
| Dtx1 | 0.0229 | 0.0842 | −0.1302 | −0.3394 |
| Apip | 0.0176 | −0.0646 | −0.0259 | 0.0911 |
| Top2a | 0.0229 | 0.0203 | −0.166 | −0.1378 |
| Gm37109 | 0.0099 | −0.0813 | 0.068 | −0.098 |
| Ptms | −0.0138 | −0.092 | 0.1541 | 0.1165 |
| Acsbg1 | −0.1175 | 0.4304 | −0.0861 | −0.1227 |
| 9230116N13Rik | 0 | −0.1147 | 0.031 | 0.3291 |
| Slco3a1 | −0.1266 | 0.4839 | −0.132 | −0.1652 |
| Gadd45b | −0.0125 | −0.0101 | −0.0622 | 0.2997 |
| Klra2 | −0.1951 | −0.143 | −0.0283 | 2.8524 |
| Adm | 0.0318 | −0.0952 | −0.0952 | −0.0952 |
| Klrc1 | −0.2791 | 0.9603 | −0.0847 | −0.2635 |
| Pgm2 | −0.0524 | 0.0952 | 0.0665 | 0.1192 |
| 5430420F09Rik | −0.029 | −0.1121 | 0.2135 | 0.2052 |
| Acaa2 | −0.0964 | 0.1222 | 0.1096 | 0.4447 |
| Igkv8-21 | 0.0013 | −0.0947 | 0.0421 | −0.0449 |
| Apol7c | 0.0149 | −0.0613 | −0.0934 | −0.0672 |
| Itih3 | −0.0882 | 0.1307 | 0.2671 | −0.1053 |
| Accs | 0.0066 | −0.0157 | 0.0585 | −0.1435 |
| Tnni2 | 0.0175 | −0.105 | 0.0749 | −0.105 |
| Pygb | 0.0375 | −0.0243 | −0.0846 | −0.1818 |
| Cd244a | −0.2198 | 0.3814 | −0.0297 | 1.1478 |
| Gm47230 | 0.0287 | −0.0783 | 0.0054 | −0.1652 |
| Man1c1 | −0.1288 | 0.2598 | −0.0631 | 0.519 |
| Gm37233 | −0.0322 | 0.0008 | −0.1056 | 0.2986 |
| Stau2 | −0.003 | −0.1417 | 0.3311 | −0.0998 |
| Pdk2 | −0.0756 | 0.0124 | 0.5116 | −0.1611 |
| Aaas | 0.0423 | −0.0568 | −0.0411 | −0.1959 |
| Lgals3bp | −0.1608 | 0.4383 | −0.2694 | 0.7002 |
| Tmem141 | −0.0658 | 0.1748 | −0.0789 | 0.051 |
| Bhlhb9 | 0.0078 | 0.0159 | −0.0976 | 0.0328 |
| Fbxo10 | 0.0096 | −0.089 | −0.0996 | 0.1177 |
| Ttll1 | 0.0012 | −0.0667 | 0.0123 | 0.2169 |
| Slc31a1 | 0.0168 | −0.0471 | −0.111 | 0.1936 |
| Sqor | −0.1503 | −0.0027 | 0.3669 | 1.0547 |
| Gpx1 | −0.0462 | −0.2898 | 0.1013 | 1.4448 |
| Dhrs3 | −0.1036 | 0.1589 | −0.1568 | 0.8885 |
| Emilin2 | −0.173 | −0.1761 | −0.1834 | 3.0135 |
| Cox6a2 | −0.0176 | −0.1021 | 0.1128 | 0.2228 |
| Tmub1 | −0.0144 | 0.1502 | −0.1415 | −0.1378 |
| Mfsd3 | 0.0122 | −0.0376 | −0.0103 | −0.0754 |
| Fbxw9 | 0.0115 | −0.0201 | −0.1267 | 0.1603 |
| Pck2 | 0.0297 | −0.1576 | 0.1387 | −0.0053 |
| Lilra5 | −0.0731 | −0.0992 | 0.251 | 0.6167 |
| Cd83 | 0.1428 | −0.435 | 0.1239 | −0.2307 |
| Ighv1-7 | 0.0445 | −0.1058 | −0.1058 | −0.1058 |
| Nat8f4 | −0.0032 | −0.1082 | 0.1847 | −0.1082 |
| Agap3 | −0.0352 | 0.083 | −0.0522 | 0.1708 |
| Socs7 | 0.0438 | −0.0454 | −0.1488 | −0.1582 |
| Ltb4r1 | −0.1001 | −0.0928 | −0.1504 | 1.789 |
| Ciao1 | −0.0293 | 0.001 | 0.0365 | 0.2668 |
| Asb2 | −0.209 | 0.6724 | −0.078 | 0.0072 |
| Trim30c | −0.0213 | 0.0723 | −0.0943 | −0.0943 |
| Pot1a | 0.0406 | −0.0276 | −0.0827 | −0.2085 |
| Gm10722 | −0.0596 | −0.0293 | 0.3597 | −0.1014 |
| Yeats2 | 0.0319 | 0.0866 | −0.2856 | −0.1607 |
| Shq1 | −0.0172 | 0.0245 | −0.114 | 0.2785 |
| Irf1 | −0.0673 | 0.1391 | −0.0336 | 0.3128 |
| Gm11168 | −0.0376 | −0.0296 | 0.1693 | −0.0081 |
| Polr3f | 0.0554 | −0.0407 | −0.1742 | −0.1588 |
| Ggt1 | −0.094 | 0.0582 | 0.3352 | 0.0101 |
| Gba2 | 0.0203 | −0.1522 | 0.1496 | 0.0622 |
| 1810043G02Rik | 0.003 | 0.1224 | −0.1789 | −0.1632 |
| Ppic | −0.0515 | 0.211 | −0.11 | −0.11 |
| Oasl2 | −0.0753 | −0.19 | −0.0274 | 1.6442 |
| Ighv5-6 | 0.0779 | −0.1429 | −0.1429 | −0.1429 |
| Nudt7 | −0.0583 | 0.0185 | 0.0801 | 0.4524 |
| Xpnpep1 | 0.0198 | −0.0489 | 0.0444 | −0.1261 |
| Gins4 | 0.0322 | −0.0607 | −0.0313 | −0.0826 |
| Tmem35b | 0.0164 | −0.0341 | 0.0717 | −0.1949 |
| Angptl4 | −0.0922 | 0.1555 | 0.29 | −0.1117 |
| Selplg | −0.4301 | 1.201 | −0.1959 | 0.7767 |
| Selenoo | −0.0121 | 0.1155 | −0.114 | −0.123 |
| Rfx2 | −0.0556 | −0.0027 | −0.0217 | 0.4714 |
| Cryzl2 | 0.0131 | −0.0942 | 0.0977 | −0.1097 |
| Igkv8-19 | 0.0194 | −0.0833 | −0.0343 | −0.12 |
| Igkv3-7 | 0.0217 | −0.1013 | −0.0272 | −0.1013 |
| Tbc1d13 | 0.0248 | −0.0153 | −0.1669 | 0.0848 |
| Ppfia4 | −0.0029 | −0.1665 | −0.1551 | 0.9578 |
| Naprt | −0.0392 | 0.0158 | 0.1242 | −0.0904 |
| Ccp110 | 0.0209 | −0.1249 | 0.136 | −0.0336 |
| Cyp4f13 | −0.0617 | 0.0024 | 0.0929 | 0.5292 |
| Mgll | −0.0459 | 0.0127 | 0.1131 | −0.0056 |
| Sesn2 | 0.0079 | −0.0773 | 0.0116 | 0.1806 |
| Asl | −0.1327 | 0.2125 | 0.2213 | 0.31 |
| Trac | −0.3828 | 1.4129 | −0.2978 | −0.3838 |
| Spata7 | 0.0077 | −0.0477 | −0.0587 | −0.1028 |
| Wrb | 0.0031 | −0.0768 | 0.2101 | −0.141 |
| Ces2a | −0.1183 | −0.0843 | 0.8364 | −0.0625 |
| Enkd1 | 0.0077 | −0.0525 | −0.0562 | −0.1047 |
| Cyp2j5 | −0.092 | −0.0729 | 0.5176 | 0.0742 |
| Abcb8 | 0.0218 | −0.098 | 0.1313 | −0.1265 |
| Fdft1 | 0.0021 | 0.0226 | −0.036 | −0.0428 |
| Cryl1 | 0.0135 | −0.1344 | −0.1595 | 0.5663 |
| Ints3 | −0.0577 | 0.1599 | −0.1414 | 0.3248 |
| Gm18422 | 0.0075 | −0.0181 | −0.0973 | −0.0973 |
| Galnt9 | −0.1145 | 0.0349 | −0.1369 | 1.3793 |
| Pex12 | 0.0412 | −0.0497 | −0.0715 | −0.1533 |
| E130309D02Rik | 0.0605 | −0.1401 | −0.1757 | 0.1449 |
| Rnf157 | 0.0369 | −0.0444 | −0.1148 | −0.0421 |
| Ighv1-54 | 0.0291 | −0.1094 | −0.0534 | −0.1094 |
| Urgcp | −0.0167 | −0.0378 | 0.168 | 0.0211 |
| Zfp866 | 0.0376 | 0.0049 | −0.1894 | −0.145 |
| Galnt6 | −0.0803 | 0.3408 | −0.2457 | 0.0948 |
| Tgm2 | −0.0886 | −0.0452 | −0.0837 | 1.1126 |
| Nipal3 | 0.0223 | 0.0456 | −0.1346 | −0.1783 |
| Ccpg1os | 0.0356 | −0.0867 | −0.1004 | −0.1004 |
| Guca1b | −0.0314 | 0.0555 | −0.1007 | 0.0485 |
| Capn3 | −0.0591 | 0.205 | −0.1092 | −0.0779 |
| Spp1 | −0.0795 | −0.0563 | 0.3828 | 0.1079 |
| Rcc1l | 0.0146 | −0.0156 | −0.0547 | −0.1222 |
| Slfn1 | −0.2731 | 0.5433 | −0.2119 | 1.4914 |
| Ncapd2 | −0.0453 | 0.1547 | 0.069 | −0.1944 |
| Ccdc114 | 0.0243 | −0.1052 | 0.003 | −0.1052 |
| Gm16299 | −0.0057 | −0.0236 | −0.0594 | −0.0844 |
| Bsn | −0.1082 | 0.2579 | −0.0233 | 0.085 |
| Zfp119b | −0.0077 | −0.079 | −0.0661 | 0.2716 |
| Serpine2 | −0.0036 | −0.0811 | 0.0819 | −0.0927 |
| Trbv16 | −0.0604 | 0.1592 | −0.0736 | −0.1013 |
| Rassf7 | −0.0086 | 0.0336 | 0.0591 | −0.1394 |
| Gm26575 | −0.008 | −0.0915 | 0.1233 | −0.0915 |
| Nckipsd | −0.0179 | −0.1225 | −0.0186 | 0.708 |
| Gpsm1 | 0.0319 | 0.012 | −0.1546 | −0.1373 |
| Slc34a1 | −0.0697 | −0.0435 | 0.3251 | 0.0633 |
| AI854703 | −0.0162 | 0.021 | −0.0677 | −0.0852 |
| Rab23 | −0.0789 | 0.087 | 0.2268 | −0.108 |
| Gapt | 0.0403 | −0.1023 | −0.0293 | −0.0219 |
| Gm43753 | −0.0124 | −0.0355 | 0.079 | −0.11 |
| Exosc5 | 0.0137 | 0.0141 | −0.0836 | −0.0551 |
| Ighv10-3 | 0.0418 | −0.1196 | −0.0897 | −0.1196 |
| Zfp511 | 0.0492 | −0.0103 | −0.2246 | −0.1072 |
| Arl11 | −0.0814 | 0.0255 | −0.0929 | 0.6563 |
| Apeh | −0.0592 | 0.2105 | 0.0296 | −0.1707 |
| Tcrg-C2 | −0.2118 | 0.7706 | −0.1233 | −0.2486 |
| Trav14-1 | −0.094 | 0.2829 | −0.0972 | −0.0972 |
| Tpm1 | −0.008 | 0.0997 | −0.1674 | 0.028 |
| Ighv2-6-8 | −0.0212 | −0.0978 | 0.2131 | −0.1063 |
| Rab7b | −0.0663 | −0.0813 | −0.0462 | 1.0503 |
| Nup93 | 0.081 | −0.1254 | −0.1377 | −0.201 |
| Elovl6 | −0.0632 | 0.1864 | −0.0947 | 0.0474 |
| Gm44130 | −0.0318 | 0.0513 | −0.0589 | −0.0854 |
| Gna15 | −0.0765 | 0.2711 | −0.0282 | −0.0931 |
| Slc13a1 | −0.0851 | −0.0665 | 0.3845 | 0.1901 |
| Miox | −0.08 | −0.054 | 0.3409 | 0.154 |
| Slc4a4 | −0.0762 | −0.0541 | 0.3409 | 0.1111 |
| Pigc | −0.0277 | 0.0468 | 0.0143 | 0.1158 |
| Ighv1-53 | 0.0858 | −0.178 | −0.1524 | −0.0451 |
| Il18rap | −0.2029 | 0.7198 | −0.0761 | −0.2462 |
| Mettl27 | −0.1071 | 0.2835 | 0.0847 | −0.1234 |
| Zswim4 | 0.0463 | −0.1858 | 0.0721 | 0.035 |
| Ighv9-4 | 0.015 | −0.0939 | −0.0428 | −0.0939 |
| BC035044 | 0.0643 | −0.1514 | −0.1946 | 0.1981 |
| Gm15564 | 0.0459 | −0.0179 | 0.0852 | −0.6231 |
| Alad | −0.0398 | 0.0284 | 0.138 | 0.0927 |
| Slc35c2 | 0.0152 | −0.2027 | −0.0209 | 0.6343 |
| Spink1 | −0.0769 | −0.0353 | 0.3369 | 0.0543 |
| Zfp553 | 0.0079 | −0.0074 | 0.0426 | −0.1739 |
| Cacna2d4 | −0.0627 | 0.1383 | 0.0837 | −0.1318 |
| C030034L19Rik | −0.1001 | 0.3158 | −0.086 | −0.1001 |
| Ryr2 | −0.0032 | 0.003 | 0.0277 | −0.1295 |
| Cald1 | −0.0318 | −0.055 | 0.1649 | −0.0901 |
| Gm37795 | 0.0083 | −0.091 | −0.0019 | −0.091 |
| Lcmt1 | −0.0137 | 0.0457 | −0.0039 | −0.0413 |
| Gm49628 | 0.0083 | −0.0515 | −0.0881 | −0.0881 |
| Dusp1 | −0.2163 | 0.3838 | −0.0686 | 1.1714 |
| Gm17334 | −0.0389 | 0.2276 | −0.1812 | −0.0746 |
| Fscn1 | 0.0067 | −0.0774 | −0.0447 | −0.0774 |
| Gm11696 | 0.015 | −0.082 | −0.082 | −0.082 |
| Abhd4 | −0.0038 | −0.1436 | −0.0757 | 0.6519 |
| Qprt | −0.1037 | 0.1947 | 0.189 | 0.0758 |
| Nnmt | −0.0208 | −0.0824 | 0.1571 | −0.0824 |
| Bend6 | −0.087 | 0.2616 | −0.087 | −0.087 |
| Laptm4b | 0.0049 | −0.0515 | −0.0826 | −0.0826 |
| Gcfc2 | −0.0338 | 0.0089 | −0.0974 | 0.3065 |
| Cdk20 | −0.0174 | 0.0219 | −0.0842 | −0.0842 |
| Myo1f | −0.3104 | 0.5666 | −0.1697 | 1.7544 |
| Igkv4-92 | −0.0313 | −0.0662 | 0.148 | −0.0662 |
| Gm45871 | 0.0051 | 0.0117 | 0.0398 | −0.1781 |
| Ints10 | −0.0593 | 0.1425 | 0.0916 | −0.0283 |
| Ei24 | −0.0274 | −0.0345 | 0.2177 | 0.0346 |
| Cldn2 | −0.0753 | −0.0753 | 0.3534 | 0.1248 |
| Crp | −0.1138 | 0.1403 | 0.3501 | 0.027 |
| Gnaz | −0.0254 | 0.0264 | −0.0594 | −0.0797 |
| Gm49439 | 0.0469 | −0.0948 | −0.1151 | −0.1151 |
| 6430562O15Rik | 0.0273 | −0.0419 | −0.1075 | −0.1207 |
| Cd3d | −0.3653 | 1.4105 | −0.3598 | −0.4605 |
| Psmf1 | 0.0266 | −0.0573 | −0.0334 | −0.0271 |
| Mgme1 | 0.0528 | −0.1497 | −0.0733 | 0.0968 |
| 4930594C11Rik | −0.0337 | 0.0645 | −0.0825 | −0.0825 |
| Kcnj15 | −0.0737 | −0.0436 | 0.3445 | −0.0093 |
| Gm37101 | −0.0691 | 0.0658 | 0.0484 | 0.4574 |
| Qars | −0.015 | 0.0462 | 0.0568 | −0.1084 |
| Pex6 | 0.0076 | −0.0634 | −0.0223 | 0.1947 |
| Tbxas1 | −0.0956 | −0.0956 | −0.0956 | 1.3985 |
| 5031425F14Rik | −0.0729 | −0.0512 | −0.0494 | 0.733 |
| Lysmd2 | −0.0339 | 0.1965 | −0.1322 | −0.1069 |
| Dpys | −0.0685 | −0.0775 | 0.3513 | 0.0486 |
| Eef2k | 0.0316 | −0.0603 | −0.0595 | −0.025 |
| Alpl | −0.0277 | −0.1087 | 0.2675 | 0.0069 |
| Gm46565 | −0.0731 | 0.0541 | −0.046 | 0.3298 |
| Tmem41a | −0.0666 | 0.1401 | −0.0719 | 0.0056 |
| Pqlc1 | 0.0594 | −0.1495 | −0.2027 | 0.2619 |
| Gc | −0.3162 | 0.0303 | 1.8727 | 0.0196 |
| Per2 | −0.0051 | 0.0808 | −0.1269 | −0.1269 |
| Tm4sf4 | −0.0659 | −0.0659 | 0.3625 | −0.0659 |
| Tpst1 | 0.0883 | −0.1748 | −0.191 | 0.002 |
| Col4a1 | −0.0751 | −0.0653 | 0.1596 | 0.4255 |
| Bpgm | 0.0777 | −0.1527 | −0.2281 | 0.1106 |
| Hmox1 | −0.0718 | −0.1203 | 0.0705 | 1.0722 |
| Uck2 | −0.0291 | 0.1521 | −0.1507 | −0.1322 |
| Cenpt | 0.0474 | −0.0388 | −0.1595 | −0.1719 |
| Ighv1-72 | −0.0128 | −0.0905 | 0.0124 | 0.2933 |
| Slc25a39 | −0.1124 | 0.1978 | 0.0907 | 0.3777 |
| Olfr433 | 0.0066 | −0.0712 | −0.0712 | −0.0712 |
| Gm45758 | −0.0319 | 0.0507 | −0.0745 | −0.0745 |
| Dcxr | 0.0093 | −0.1512 | 0.1158 | 0.2493 |
| Ctsf | −0.0671 | 0.0466 | −0.0712 | 0.3317 |
| Gm33195 | 0.0043 | −0.0205 | −0.0924 | −0.0924 |
| Epb41l1 | −0.0683 | 0.1568 | −0.0714 | −0.0714 |
| P4htm | −0.0488 | 0.2487 | −0.1388 | −0.1388 |
| Irf7 | −0.1264 | 0.0634 | 0.0673 | 1.0941 |
| Rcbtb2 | −0.0941 | 0.3236 | −0.0955 | 0.0378 |
| Gabrb3 | −0.0314 | −0.0662 | 0.148 | −0.0662 |
| Dmpk | −0.0256 | −0.085 | −0.0982 | 0.7097 |
| Gm49927 | −0.0314 | 0.0398 | −0.0662 | −0.0662 |
| Ldlr | −0.1176 | 0.1155 | −0.2437 | 1.3788 |
| Arl6 | −0.0238 | 0.029 | −0.077 | −0.077 |
| Gm5113 | −0.0072 | 0.0242 | −0.0915 | −0.0915 |
| Ccdc166 | −0.0236 | 0.1642 | −0.1413 | −0.0887 |
| Elovl1 | −0.0316 | 0.1299 | −0.1573 | 0.1673 |
| Fam219b | −0.053 | 0.178 | 0.1096 | −0.2701 |
| Slc25a14 | −0.0071 | 0.0306 | −0.0132 | −0.0646 |
| Efhd2 | −0.215 | 0.5936 | −0.2466 | 0.6936 |
| mt-Co1 | −0.2304 | 0.0431 | 1.5638 | −0.4403 |
| Sh3bgrl2 | −0.0448 | 0.0233 | −0.0828 | 0.3205 |
| Tmem161a | −0.023 | −0.0055 | 0.0172 | 0.2538 |
| Gm38091 | −0.0384 | −0.0323 | 0.1316 | −0.0547 |
| C8b | −0.0952 | 0.1156 | 0.3168 | −0.0952 |
| Ubap1 | −0.0412 | 0.1242 | 0.0412 | −0.0732 |
| Ighv1-4 | 0.0375 | −0.1163 | −0.0022 | −0.1163 |
| Igkv4-68 | 0.1108 | −0.1857 | −0.1786 | −0.2393 |
| Gas2l1 | −0.0245 | −0.0745 | −0.0745 | 0.3285 |
| Bad | −0.0271 | −0.0081 | 0.0407 | 0.2677 |
| Ighv1-18 | 0.0926 | −0.212 | −0.1829 | 0.0464 |
| Gm42728 | 0.0148 | −0.0089 | −0.1154 | −0.1154 |
| Phc2 | −0.077 | 0.0152 | 0.205 | 0.4468 |
| D730003I15Rik | −0.0249 | 0.1161 | −0.1082 | −0.1097 |
| Sirt4 | 0.0509 | −0.064 | −0.1498 | −0.1498 |
| Gm43633 | 0.0034 | 0.076 | −0.1326 | −0.1326 |
| Arhgap6 | 0.0002 | −0.1156 | 0.1812 | −0.1008 |
| Atp8a2 | −0.2388 | 0.8435 | −0.1013 | −0.2543 |
| Zfp61 | 0.029 | −0.017 | −0.1125 | −0.0716 |
| Ighv1-52 | 0.0785 | −0.1235 | −0.1521 | −0.1521 |
| Crat | 0.0298 | −0.08 | −0.1403 | 0.2055 |
| Slco4a1 | 0.0071 | −0.02 | 0.0481 | −0.096 |
| Slc22a1 | −0.1036 | −0.0299 | 0.6323 | −0.0073 |
| Nsun4 | 0.0514 | −0.0447 | −0.1041 | −0.2284 |
| Uso1 | −0.0242 | 0.0946 | −0.238 | 0.3681 |
| Rom1 | −0.042 | 0.1335 | −0.1057 | −0.0387 |
| Ighv1-67 | 0.0004 | −0.102 | 0.154 | −0.102 |
| Nfkbil1 | −0.0454 | 0.1576 | −0.0365 | −0.0053 |
| Mad2l1bp | 0.0134 | −0.0329 | 0.0232 | −0.0739 |
| Alms1 | 0.0112 | −0.0047 | −0.0527 | −0.1235 |
| Tufm | 0.0713 | −0.0824 | −0.2117 | −0.1131 |
| Ces1f | −0.079 | −0.0098 | 0.2657 | 0.2883 |
| Cd300lb | −0.1354 | −0.0741 | −0.0982 | 1.9898 |
| Dhodh | −0.0114 | −0.0253 | 0.2232 | −0.1914 |
| Zfp346 | 0.0054 | 0.0804 | −0.1224 | −0.1376 |
| Igkv4-57 | 0.0795 | −0.1749 | −0.0746 | −0.1155 |
| Stap2 | −0.0637 | −0.0693 | 0.3592 | −0.0693 |
| Adipor1 | −0.0902 | 0.0872 | −0.1103 | 0.919 |
| Prmt7 | 0.0624 | −0.1608 | −0.0689 | 0.0194 |
| Gmnn | 0.0242 | 0.0229 | −0.1735 | −0.04 |
| Acot11 | −0.0551 | 0.0589 | −0.0502 | 0.3719 |
| Adck1 | −0.0359 | 0.1055 | 0.0948 | −0.1851 |
| Coprs | −0.0046 | −0.0652 | −0.092 | 0.1793 |
| Gm48998 | −0.0308 | −0.0657 | −0.0657 | 0.337 |
| Birc5 | −0.054 | 0.1109 | −0.014 | 0.0048 |
| Slc25a10 | −0.0311 | 0.0156 | 0.0338 | 0.2367 |
| Hhat | 0.0446 | −0.1499 | −0.0252 | 0.0417 |
| Mbd3 | 0.0131 | −0.0367 | −0.0241 | 0.0335 |
| Slc22a12 | −0.0117 | −0.0842 | 0.1304 | −0.057 |
| Sdc1 | 0.0295 | −0.0449 | 0.0081 | −0.1857 |
| Abt1 | 0.0144 | −0.0377 | 0.1392 | −0.2844 |
| Clec10a | −0.0502 | −0.1127 | 0.0919 | 0.8167 |
| Ighv1-69 | 0.1024 | −0.1886 | −0.1829 | −0.1228 |
| Txnrd3 | −0.0131 | 0.1097 | −0.11 | −0.11 |
| Zfp870 | 0.0706 | −0.1377 | −0.1377 | −0.0731 |
| Extl1 | 0.054 | −0.1267 | −0.0535 | −0.1413 |
| Igkv2-109 | 0.1237 | −0.2607 | −0.077 | −0.2947 |
| Gm33023 | −0.0449 | 0.017 | 0.1681 | −0.1012 |
| Arc | −0.0477 | 0.1072 | 0.1025 | −0.1667 |
| Hspbp1 | −0.0153 | 0.1547 | −0.0499 | −0.3179 |
| Mrm2 | 0.0101 | −0.0113 | −0.1033 | −0.1033 |
| Npc2 | −0.1649 | 0.1678 | −0.2169 | 1.6765 |
| Fxyd2 | −0.0635 | −0.0701 | 0.335 | 0.0737 |
| Got1 | 0.0068 | 0.0375 | 0.0215 | −0.2613 |
| Traip | 0.0175 | 0.0011 | −0.0807 | −0.1261 |
| Faap100 | 0.0025 | −0.114 | 0.0789 | 0.2563 |
| Usp11 | 0.049 | −0.0353 | −0.1748 | −0.1515 |
| Nubpl | −0.0218 | 0.0573 | 0.0631 | −0.1306 |
| Spryd7 | 0.0086 | 0.0028 | −0.002 | −0.1685 |
| Zfp568 | 0.0357 | −0.0621 | −0.0877 | −0.0775 |
| Nup107 | 0.0332 | −0.1055 | 0.0133 | −0.008 |
| Rhoc | −0.1459 | 0.5137 | −0.0891 | −0.0974 |
| Paqr7 | −0.1243 | 0.3449 | −0.0469 | 0.2143 |
| Pkd1l2 | −0.0279 | −0.0605 | 0.1332 | −0.0813 |
| Igkv15-103 | 0.0522 | −0.1098 | −0.1166 | −0.1166 |
| Thbs3 | −0.024 | −0.0938 | 0.1209 | 0.3099 |
| Srpk3 | 0.2094 | −0.4368 | −0.281 | −0.2321 |
| Gm42466 | 0.0058 | −0.0432 | −0.0399 | −0.0725 |
| Endov | 0.0216 | 0.0803 | −0.1681 | −0.2389 |
| Mtg2 | −0.0138 | 0.1207 | −0.1216 | −0.0704 |
| Rpn1 | −0.0476 | 0.0754 | −0.1261 | 0.5005 |
| Tmem26 | −0.0209 | −0.141 | 0.1237 | 0.4995 |
| Tmem177 | 0.0271 | −0.0992 | 0.1241 | −0.1702 |
| Fcgr3 | −0.2208 | 0.0622 | −0.2109 | 2.7124 |
| mt-Nd4l | −0.2282 | 0.0723 | 1.3655 | −0.2045 |
| Ptprf | −0.0841 | −0.06 | 0.4179 | 0.2857 |
| Prnp | 0.0083 | −0.0567 | −0.0536 | −0.1085 |
| Mettl3 | −0.0066 | 0.0518 | −0.2098 | 0.2734 |
| Gm43201 | −0.0279 | 0.0349 | −0.0711 | −0.0711 |
| Pja1 | −0.0023 | −0.0134 | 0.1557 | −0.2158 |
| Gale | −0.0476 | 0.0214 | 0.1298 | −0.0848 |
| Clnk | −0.149 | 0.4408 | 0.0797 | −0.1024 |
| Igkv6-25 | 0.0017 | −0.1466 | 0.2485 | 0.0092 |
| 2610035D17Rik | 0.0294 | −0.002 | 0.0473 | −0.4215 |
| Fes | −0.1233 | −0.0532 | −0.2495 | 2.0967 |
| Ric8a | −0.0354 | −0.0572 | 0.1097 | 0.4198 |
| Trbv31 | −0.0858 | 0.2989 | −0.1091 | −0.1091 |
| Trim41 | −0.0176 | −0.003 | 0.0939 | 0.0385 |
| Mier2 | −0.0123 | 0.1094 | −0.1341 | −0.0211 |
| Hmgcl | −0.074 | 0.0704 | −0.0564 | 0.6942 |
| Trabd | 0.0156 | −0.0544 | −0.0837 | 0.1837 |
| Gh | 0.0477 | −0.1064 | −0.0653 | −0.1064 |
| Itpkc | −0.0254 | 0.0374 | −0.0787 | −0.0787 |
| Mical3 | 0.0324 | −0.0954 | 0.0378 | −0.0829 |
| Map7d1 | −0.048 | 0.067 | −0.133 | 0.5503 |
| Crisp3 | 0.037 | −0.1168 | −0.006 | −0.1168 |
| Slc25a15 | −0.0387 | −0.1118 | 0.4952 | −0.1232 |
| Caprin2 | 0.0336 | −0.0295 | −0.1301 | −0.1301 |
| Soga1 | −0.0613 | −0.0718 | −0.0859 | 0.9713 |
| Slc7a2 | −0.0446 | −0.0877 | 0.3145 | −0.0877 |
| Bud13 | 0.0123 | −0.1028 | 0.0224 | 0.2059 |
| Upb1 | −0.0014 | −0.1247 | 0.0907 | 0.2535 |
| Zmat3 | −0.024 | 0.124 | −0.0658 | −0.1202 |
| Pex16 | 0.0128 | 0.0064 | −0.0364 | −0.1039 |
| Cxcr5 | 0.2345 | −0.4992 | −0.1846 | −0.4671 |
| Hck | 0.0349 | −0.4246 | −0.1553 | 1.5022 |
| C4b | −0.16 | −0.0642 | 1.1342 | −0.0435 |
| Gm2447 | 0.0196 | −0.1316 | 0.0842 | −0.0408 |
| Asb8 | −0.0164 | −0.0266 | −0.04 | 0.3664 |
| Pak6 | −0.0985 | 0.2801 | −0.0317 | −0.0985 |
| Smpdl3b | −0.1105 | −0.0818 | −0.1201 | 1.7878 |
| Klra1 | −0.1244 | 0.4491 | −0.0822 | −0.1138 |
| Mefv | −0.0676 | −0.1026 | −0.1026 | 1.2411 |
| St6galnac4 | 0.0092 | 0.0039 | −0.1619 | 0.1838 |
| Palm3 | −0.0684 | 0.137 | 0.1397 | −0.0722 |
| Klrd1 | −0.3003 | 1.051 | −0.2179 | −0.1125 |
| Ugt2b36 | −0.1046 | 0.2248 | 0.1428 | −0.1091 |
| Armc6 | −0.0261 | 0.0164 | 0.1125 | −0.0617 |
| Shcbp1 | 0.0276 | −0.0949 | −0.0949 | −0.0949 |
| Gatd1 | 0.0225 | −0.1019 | 0.1285 | −0.114 |

### REFERENCES (EXAMPLE 1)

1. Haque, A., Engel, J., Teichmann, S. A. & Lönnberg, T. A practical guide to single-cell RNA-sequencing for biomedical research and clinical applications. Genome Med 9, 75 (2017).
2. Kolodziejczyk, A. A., Kim, J. K., Svensson, V., Marioni, J. C. & Teichmann S A. The technology and biology of single-cell RNA sequencing. Mol Cell. 58, 610-620. (2015).
3. Earl, C. C., Smith, M. T., Lease, R. A., & Bundy, B. C. Polyvinylsulfonic acid: A Low-cost RNase inhibitor for enhanced RNA preservation and cell-free protein translation. Bioengineered. 9, 90-97. (2018).
4. Smyrlaki, I. et al. Massive and rapid COVID-19 testing is feasible by extraction-free SARS-CoV-2 RT-PCR. Nat Commun 11, 4812 (2020).
5. Picelli, S., Bjorklund, Å., Faridani, O., Sagasser, S., Winberg, G., & Sandberg, S. Smart-seq2 for sensitive full-length transcriptome profiling in single cells. Nat Methods 10, 1096-1098 (2013).
6. Johnston, A. D., Lu, J., Ru, K I., Korbie, D., & Trau, M. PrimerROC: accurate condition-independent dimer prediction using ROC analysis. Sci Rep 9, 209 (2019).
7. The Tabula Muris Consortium. et al. Single-cell transcriptomics of 20 mouse organs creates a Tabula Muris. Nature 562, 367-372 (2018).
9. Park, J. et al. Single-cell transcriptomics of the mouse kidney reveals potential cellular targets of kidney disease. Science. 360, 758-763. (2018).
10. MacParland, S. A. et al. Single cell RNA sequencing of human liver reveals distinct intrahepatic macrophage populations. Nat Commun 9, 4383 (2018).
11. Hagemann-Jensen, M. et al. Single-cell RNA counting at allele and isoform resolution using Smart-seq3. Nat Biotechnol 38, 708-714 (2020).

### EXAMPLE 2—COATING OF FIBROUS MATERIAL USING POLYVINYL SULFONIC ACID ENABLES EFFICIENT SAMPLING AND STORAGE OF RNA MATERIAL FOR DOWNSTREAM CDNA LIBRARY GENERATION

A major application in clinical as well as non-clinical setting for this class of chemical RNase inhibitors could be to treat a solid substrate, such as a paper sheet with the chemical RNase inhibitor; forming a coated material that could be used for preserving RNA sample material at room temperature for extended periods of time. Such a sampling substrate material could minimize storage space, greatly reduce energy usage, and prevent freeze-thaw degradation of fragile biological material. Here we demonstrate the proof of principle of such a material.

### Results

We performed a paper-spotting assay with fiber sheets pre-incubated in solutions containing a range of PVSA concentrations (30 μg/mL, 300 μg/mL, 3 mg/mL) as well water only as control. After incubating the paper-strip material in PVSA solution, we added the equivalent of 500 lyzed mouse liver cells to its surface (10 mm×10 mm area), let it dry, and then incubate at room temperature for 24 hours. After 24 hours, sample material was eluted from the paper strip using water, and a volume estimated to contain the equivalent of approximately 5 cells worth of input sample material was used as RNA input for Smart-seq2 cDNA synthesis reactions. Resulting cDNA was purified and inspected for cDNA yield and size distribution using capillary electrophoresis (Bioanalyzer 2100, Agilent). We identified that increased PVSA concentration in the paper-strip material drastically improved the yield of recovered cDNA and as well as increased its average fragment size, demonstrating RNA preservation in paper-strip material. 10 mg/mL PVSA-incubated paper strips had the greatest RNA preservation capability, while 100 μg/mL and water only incubations resulted in almost complete RNA degradation (FIG. 10). This demonstrates a novel storage procedure for RNA-containing samples that can be used in downstream cDNA library synthesis.

### Methods

Western blotting cotton fiber paper (iBlot Filter Paper) was pre-incubated in 30 μg/mL, 300 μg/mL, or 3 mg/mL PVSA, or water only for 5 minutes, air dried for 15 minutes, then cut into approximately 10 mm×10 mm pieces and stored in Eppendorf tubes until used. A pellet of approximately 3 million mouse liver cells was diluted to 200 cells/μL in 1% Triton-X, and the paper was spotted with 2.5 μL of the cell suspension, then air dried for 15 minutes. The paper material with deposited liver-cell material was kept at room temperature for 24 hours and then eluted in 100 μL of water. We then generated cDNA with the standard Smart-seq2 protocol (Picelli 2013) using 1 μL of elute and 22 cycles for cDNA amplification. The resulting cDNA libraries were analyzed using Bioanalyzer 2100 (Agilent) High sensitivity chips.

### REFERENCES (EXAMPLE 2)

Picelli, S., Bjorklund, Å., Faridani, O. et al. Smart-seq2 for sensitive full-length transcriptome profiling in single cells. Nat Methods 10, 1096-1098 (2013). https://doi.org/10.1038/nmeth.2639

### EXAMPLE 3—PROLONGING WORKABLE STORAGE TIME FOR SINGLE-CELL RNA MATERIAL IN LYSIS BUFFER USING POLYVINYL SULFONIC ACID

### Introduction

Due to the fragile nature and miniscule amount of RNA material in single cells in combination with the abundance of RNase from tissue material and external sources, recombinant RNase inhibitor (RI) is generally supplied to single-cell RNA-seq lysis buffers used for cellular capture and storage. However, conventional recombinant RNase inhibitors, being proteins, are thermosensitive and tend to lose their efficacy to inhibit RNases when kept above freezing temperature for prolonged time. This poses several constraints on single-cell RNA-seq workflows. Cells sorted into lysis buffer in PCR plates or other collection vessels need to be frozen shortly after cell collection and the lysis buffer plates themselves need to be either freshly spotted with lysis buffer before cell collection or kept or delivered in frozen condition. This raises barriers in workflows and cross-laboratory collaboration. For example, laboratories having access to and performing cell sorting and collection from relevant tissues (e.g. patient and animal material) are often not the same as the laboratory having expertise in single-cell RNA-seq library preparation. If the workable storage timeframe after single-cell collection could be extended this would drastically facilitate single-cell RNAs-seq workflows. Here we show that using polyvinyl sulfonic acid (PVSA) in single-cell lysis buffer can extend such workable timeframes.

### Results and Discussion

Given our findings that specific concentration ranges of PVSA in cell lysis buffer can effectively replace recombinant RNase inhibitor in single-cell RNA-seq library preparation without negatively affecting the resulting sequencing library quality (Example 1), we decided to investigate whether PVSA might additionally be able to prolong the workable storage time of collected RNA material. Here, we tested the capacity of PVSA to preserve collected single-cell or extracted RNA in solution, stored at room temperature (25° C.) or in refrigerator (4° C.) for extended time.

As a starting point, we added variable amounts of PVSA into low-retention Eppendorf tubes together with purified total RNA in water extracted from cultured human embryonic kidney cells (HEK293FT). The final PVSA concentrations in the conditions investigated were 0, 0.12, 0.3, 0.6, and 1.2 mg/mL and the total RNA concentration was kept at 20 ng/μL in each condition. In parallel we prepared a sample containing 10 Units/μL recombinant RNase inhibitor (cat. no. 2313B, TaKaRa) along with the 20 ng/μL total RNA. The RNA samples were kept at room temperature (25° C.) and we collected aliquots from each condition after 0, 3, 7, 14, and 21 days in storage. The collected aliquots were then subjected to ribosomal RNA band inspection using Bioanalyzer 2100 RNA 6000 Nano chips (Agilent). As expected, we observed distinct ribosomal RNA bands (28S and 18S) in samples from all conditions at the starting time point (day 0) followed by a storage-time dependent degradation of ribosomal RNA, observable by diminishing 28S:18S band intensity (FIG. 11a). Compared to PVSA conditions, ribosomal RNA degradation progressed notably faster in the sample containing no inhibitor (0 mg/mL PVSA) likewise did the sample with conventional protein-based recombinant RNase inhibitor (FIG. 11a-e). In the PVSA conditions, we observed superior RNA quality and importantly a PVSA-concentration dependent inhibition of ribosomal RNA degradation (FIG. 11a-e). Indeed, we observed distinct dual 28S+18S ribosomal RNA bands even after 21 days of storage at room temperature for RNA stored with the highest PVSA concentration. To also investigate the intactness of messenger RNA (mRNA), we performed bulk-RNA Smartseq2 cDNA synthesis with full-transcript-length amplification on aliquots of each RNA condition and day (Methods) and analyzed the resulting cDNA fragment-size distribution and yield on Bioanalyzer 2100 dsDNA HS chips (Agilent). In line with our observations on ribosomal RNA bands, intact cDNA distributions from bulk RNA were generated for all conditions at day 0 (FIG. 11f). Upon prolonged storage, RNA samples in 0 mg/mL PVSA condition (no inhibitor) and the recombinant RNase inhibitor condition resulted in cDNA libraries with fragment sizes shifted towards shorter lengths and diminishing height of the characteristic cDNA peak at around 2 kb present in intact full-length cDNA libraries (FIG. 11f-j), indicating mRNA degradation in these conditions. In the PVSA conditions on the other hand, cDNA libraries were markedly more intact, producing similar cDNA-fragment-size distributions throughout day 0-21. Samples containing the highest amount of PVSA produced libraries of intact size distribution but with lower cDNA yield, which was expected due to reaction inhibition upon overly high PVSA concentration during library synthesis (Example 1). Together, this experiment demonstrated that inclusion of PVSA in RNA samples stored in water decreases ribosomal RNA and messages RNA degradation rate at room temperature in a PVSA-concentration-dependent manner.

Encouraged by these results, we next tested whether including PVSA in single-cell lysis buffer could improve RNA integrity of collected single cells stored over time. We sorted individual cultured human embryonic kidney cells (HEK293FT) by FACS into wells containing Smart-seq2 lysis buffer in which we had replaced recombinant RNase inhibitor with 0, 60, 90, and 120 μg/mL PVSA (Methods). We earlier identified this to be in the optimal working range for PVSA in the Smart-seq2 protocol (Example 1). The cell samples were processed into Smart-seq2 cDNA libraries on the same day (day 0) or after 1, 4, 7, 14 days stored at room temperature (25° C.), refrigerator (4° C.), or at −80° C. (n=24 single-cell libraries per condition). We analyzed the resulting cDNA libraries from each condition on Bioanalyzer 2100 dsDNA HS chips (Agilent). As expected, we observed that cells stored in lysis buffer lacking inhibitor resulted in degraded libraries, reflected by shifts towards smaller-sized cDNA fragments in the Bioanalyzer traces (FIG. 12). Also expected, given our earlier findings (Example 1), single-cell samples kept in lysis buffer containing 60-120 μg/mL PVSA produced intact cDNA profiles from day-0 samples and −80° C. (FIG. 12a-b). More intriguingly, acceptable single-cell cDNA profiles were yielded from PVSA samples even at 7 days of storage in room temperature (25° C.) (FIG. 12c) and remarkably throughout the full 14-day experiment when stored in refrigerator (4° C.) (FIG. 12d). Together, this shows that PVSA has the capacity to protect RNA of single cells in lysis buffer for prolonged times in convenient and easily workable storage conditions (room temperature and fridge).

To systematically characterize the capacity of PVSA to retain quality of long-term stored single-cell RNA, we conducted a large-scale sequencing experiment utilizing the newly developed Smart-seq3xpress protocol (1). This protocol is a miniaturized adaptation of Smart-seq3 (2), that is different to Smart-seq3 not only by a lowered reaction volume but also in that the protocol completely circumvents the cDNA bead purification step, instead proceeding with tagmentation and indexing PCR immediately after a cDNA dilution step. We spotted 384-well PCR plates with Smart-seq3xpress lysis buffer containing variable concentration of PVSA (0, 0.6, 1.5, 3.0, 15, or 30 μg/mL) as well as normal Smart-seq3xpress lysis buffer containing conventional recombinant RNase inhibitor in accordance with the original protocol (n=96 wells per condition across nine 384-well plates). We sorted individual cultured human embryonic kidney cells (HEK293FT) by FACS into the wells of the PCR plates and sealed the plates. Two plates were put to −80° C. immediately after cell sorting (representing the day 0 time point) while the other plates were stored at either room temperature (25° C.) or in refrigerator (4° C.). Plates containing cells and the various inhibitor conditions were collected after 1, 4, or 7 days in case of 25° C. storage; and after 1, 4, 7, and 14 days in case of 4° C. storage; and processed into Smart-seq3xpress sequencing libraries and finally sequenced (Methods). After read mapping to the human genome, we identified the optimal concentration range of PVSA for the Smart-seq3xpress protocol using read mapping statistics of the various conditions on the day 0 plates, which was found to be 0.5-3.0 μg/mL in the lysis buffer, i.e. similar to what we earlier identified for full-volume Smartseq3 (FIG. 13a-d). PVSA concentration of 6.0 μg/mL in the lysis buffer produced lower-yield libraries, while 15 and 30 μg/mL PVSA in the lysis buffer produced unusable libraries. Thus, the optimal concentration PVSA range Smart-seq3xpress was established. Next, we analyzed the data from cells and plates stored at 25° C. and 4° C., considering only the workable PVSA conditions (0.5-6.0 μg/mL), no inhibitor condition (0 μg/mL PVSA), and standard Smart-seq3xpress with recombinant RNase inhibitor. As sequencing depth affects gene detection and number of unique molecular identifiers (UMIs) detected in sequencing libraries, we down sampled the datasets to the same read depth (100,000 reads per cell) making conditions directly comparable. As expected, we observed the overall trend of decreasing number of genes and UMIs detected per cell over time in storage, with a more rapid decrease with time in 25° C. than in 4° C. (FIG. 13e-h). Interestingly, PVSA conditions 0.5-3.0 μg/mL consistently produced better libraries (more genes and UMIs detected per cell) that standard Smart-seq3xpress using recombinant RNase inhibitor within each day investigated, including day 0. This indicates that PVSA may improve the miniaturized Smart-seq3xpress over the normal protocol which uses recombinant RNase inhibitor. Even more interesting, we observed that PVSA conditions 0.5-3.0 μg/mL stored up to 4 days in room temperature produced libraries with quality superior to or on par with the recombinant RNase inhibitor at day 0. Consistently, cells stored with recombinant RNase inhibitor displayed more progressed degradation over time. Remarkably, for single cells stored at 4° C., we found that the number of genes detected in PVSA conditions 0.5-3.0 μg/mL were superior to the recombinant RNase inhibitor condition even after two weeks of storage (day 14) (FIG. 13g). Intriguingly, the number of genes detected in PVSA condition 6.0 μg/mL remained nearly unchanged across day 0 and day 14, indicative of PVSA-concentration-dependent preservation of single-cell RNA, similarly to what we found in our initial bulk RNA experiments on the level of ribosomal RNA (FIG. 11).

Together, we conclude that substituting recombinant RNase inhibitor with PVSA in single-cell lysis buffer can extend the workable time frame for processing of lysed cells into RNA-sequencing libraries. This has several important practical implications to the single-cell RNA-seq workflow. For example, by using PVSA in single-cell lysis buffer, sorted cell plates be stored and transferred in refrigerated condition rather than at −80° C. or dry ice which currently is the established procedure, thereby reducing cost and energy consumption. This benefit of PVSA in single-cell RNA-sequencing workflows does not only simplify laboratory procedures (e.g. by allowing cells collected in lysis buffer to be stored in fridge instead of −80° C. and proceed with library prep on a following day) and cross-laboratory transfer of samples, but could enable new experimental procedures requiring RNA to stay intact in solution while additional experimentation is performed on the cell material. An example of this is tagmentation of nuclear DNA, e.g. for genomic DNA sequencing or for assay for transposase-accessible chromatin with sequencing (ATAC-Seq) combined with single-cell RNA-seq. Because of the thermostability of PVSA, it could be possible to tagment the nuclear DNA of a cell using Tn5, then inactivate Tn5 by heating the sample yet keeping PVSA intact, and subsequently proceed with cDNA synthesis after the thermal Tn5 inactivation. This would allow combined single-cell DNA/RNA-sequencing of a cell without the need to physically separate the nucleus (containing DNA) and the lysed cytoplasm (containing RNA). Thermostability of PVSA will furthermore allow manufacturing of premade plates and other vessels carrying single-cell lysis buffer (containing PVSA and a detergent; optionally deoxynucleoside triphosphates [dNTPs] and cDNA synthesis primers) which can be included in various kits and products for single-cell RNA-seq library preparation.

### Methods

### Cell Culture and Sorting

HEK293FT cells (Invitrogen) were grown in DMEM medium (4.5 g L-1 of glucose and 6 mM L-glutamine, Gibco), supplemented with 10% FBS (Sigma-Aldrich), 0.1 mM MEM non-essential amino acids (Gibco), 1 mM sodium pyruvate (Gibco) and 100 pg ml-1 of penicillin-streptomycin (Gibco) at 37° C. Before sorting cells were detached using TrypLE Express (Gibco), and washed with PBS. Cells were stained with propidium iodide (Thermo Fisher Scientific) to exclude dead cells and single cells were sorted into 384-well plates containing lysis buffer using a BD FACSMelody (BD FACSChorus version 1.3 software) equipped with a 100-μm nozzle and plate cooling with index sorting on (BD Biosciences). After sorting, each plate was immediately spun down and stored at −80° C.

### RNA Storage Buffer Experiment

HEK293FT RNA was purified using the Qiagen RNeasy Mini Purification kit (cat. 74104, Qiagen), and 400 ng RNA was added to a total volume of 20 pl storage buffer (20 ng/μl for the RNA), containing either no inhibitor (0 mg/mL), PVSA (0.12, 0.3, 0.6, or 1.2 mg/mL), or 10 U/μl Recombinant RNase inhibitor (cat. 2313B, TaKaRa). Aliquots of 1 ul at 0, 3, 7, 14, and 21 days for were removed to run on an Bioanalyzer RNA 6000 Nano Chip (Agilent). Aliquots of 0.4 pl (8 ng RNA) were collected at 0, 3, 7, 14, and 21 days for cDNA library generation according to a slightly modified version of the Smart-seq2 (3) protocol where no additional recombinant inhibitor was added to the lysis buffer, resulting in in concentrations of 0, 11, 27, 54, or 106 μg/mL of PVSA or 0.9 U/μl of RRI (cat. no. 2313B, Recombinant RNase Inhibitor, TaKaRa) for each sample and a final Smart-seq2 lysis buffer composition of 0.1% Triton X-100, 2.5 mM dNTP/each (cat. no 10297018, Invitrogen), and 2.5 mM Smart-seq2 oligo-dT (5′-AAGCAGTGGTATCAACGCAGAGTACT30VN-3′, IDT) in a 4.5 pl total solution. Samples were then incubated at 72° C. for 3 min. Reverse transcription mix was added which contained 1× Superscript II buffer, 5 mM DTT, 1M betaine (cat. B0300, Sigma), 10 mM MgCl₂, 1 uM Smart-seq2 TSO (5′-AAGCAGTGGTATCAACGCAGAGTACATrGrG+G-3′, IDT), 10 U Superscript II (cat. no. 18064-014, Invitrogen), and the lysis buffer mix in a total volume of 10 pl. During the reverse transcription step, only the Recombinant RNase inhibitor (RI) sample was supplemented with additional recombinant inhibitor (cat. no. 2313B, Recombinant RNase Inhibitor, TaKaRa) in accordance with the original Smart-seq2protocol and for all other samples the addition of recombinant inhibitor was omitted. Reverse transcription thermocycles were 42° C. for 90 min, followed by 10 cycles of 42° C. for 2 min and 50° C. for 2 min. The cDNA amplification reaction mixture composition was 1× KAPA HiFi HotStart Ready Mix (cat. no 07958935001, Roche Diagnostics), 80 nM ISPCR primers (5′-AAGCAGTGGTATCAACGCAGAGT-3′, IDT) and the 10 pl first strand cDNA in a total volume of 25 pl. The cDNA was then amplified as follows: 98° C. for 3 min, then 12 cycles of 98° C. for 20 sec, 67° C. for 15 sec, and 72° C. for 6 min, followed by a final incubation at 72° C. for 5 min. Amplified cDNA was bead-purified (AMPure XP, Beckman Coulter) and run on High Sensitivity DNA chips using a Bioanalyzer 2100 (Agilent) to assess cDNA quality.

### Smart-Seq2 Time Experiment

HEK293FT cells were FACS-sorted into Smart-seq2 lysis buffer (0.1% Triton X-100, 2.5 mM dNTP/each, and 2.5 mM Smart seq2 oligo-dT), containing 0, 60, 90, or 120 μg/mL of PVSA in 96 well plates, and were subsequently stored at either 25° C., 4° C., or −80° C. After 1, 4, 7, or 14 days, two replicas of each plate storage condition were processed for cDNA following the Smart-seq2 protocol (omitting the biological inhibitor in the reverse transcriptase step as described in the previous section) with 20 cycles of PCR amplification. An additional two plates were immediately processed into cDNA following the Smart-seq2 protocol after sorting as a control (day 0). After cDNA library amplification, samples were bead purified (AMPure XP, Beckman Coulter) and run on High Sensitivity DNA chips using a Bioanalyzer 2100 (Agilent) to assess cDNA quality.

### Smart-Seq3Xpress Time Experiment and Library Preparation

Smart-seq3xpress libraries were performed as previously described¹ with some modifications. In brief, cells were sorted into 384 well plates each containing 3 uL Vapor-Lock (Qiagen) in all wells and eight different conditions of 0.3 μL lysis buffer consisting of 0.125 μM OligodT30VN (5′-Biotin-ACGAGCATCAGCAGCATACGAT₃₀VN-3′; IDT) adjusted to reverse transcription (RT) volume, 0.5 mM dNTPs/each adjusted to RT volume, 0.1% Triton X-100, 5% PEG8000 adjusted to RT volume, and the indicated type and amount of RNase Inhibitor (no RNAse inhibitor, 0.4p RNase Inhibitor (Takara Bio, 40 U uL-1), PVSA; 0.6 μg/ml, 1.5 μg/ml, 3.0 μg/ml, 6 μg/ml, 15 ug/ml, 30 μg/ml). After cell sorting plates were briefly centrifuged before put to −80° C. To test the effect of temperature and time on single cell RNA stability for each of the 8 lysis conditions, sorted 384 well plates containing sorted cells in lysis buffer were left at either room temperature (25° C.) for 1, 4, or 7 days or in fridge (4° C.) for 1, 4, 7, or 14 days, before commencing library preparation. To serve as control, two plates were extracted immediately from −80° C. immediately before library preparation.

Before RT, plates were denatured at 72° C. for 10 min followed by addition of 0.1 μL of RT mix; 25 mM Tris-HCL pH 8.4 (Fischer Scientific), 30 mM NaCl (Ambion), 1 mM GTP (Thermo Fisher Scientific), 2.5 mM MgCl₂ (Ambion), 8 mM DTT (Thermo Fisher Scientific), 0.75 uM Template Switching Oligo (TSO) (5′-Biotin-AGAGACAGATTGCGCAATGNNNNNNNNWWrGrGrG-3′; IDT) and 2 U pl-1 of Maxima H Minus reverse transcriptase (cat. EP0752, Thermo Fisher Scientific). Plates were quickly centrifuged after dispensing to ensure merge of lysis and RT volumes underneath the Vapor-lock overlay and incubated at 42° C. for 90 minutes, followed by ten cycles of 50° C. for 2 minutes and 42° C. for 2 minutes. After RT, 0.6 μL PCR mix was dispensed to each well containing the following: 1× SeqAmp PCR buffer (Takara Bio), 0.025 U pl-1 of SeqAmp polymerase (Takara Bio) and 0.5 μM Smartseq3 forward (5′-TCGTCGGCAGCGTCAGATGTGTATAAGAGACAGATTGCGCAATG-3′; IDT) and reverse primer (5′-ACGAGCATCAGCAGCATACGA-3′; IDT). Plates were quickly spun down before being incubated as follows: 1 minute at 95° C. for initial denaturation, 12 cycles of 10 seconds at 98° C., 30 seconds at 65° C. and 4 minutes at 68° C. Final elongation was performed for 10 minutes at 72° C.

After PCR, pre-amplified libraries were diluted with 9 μL H2O, before transferring 1 μL of diluted cDNA from each well into a new 384 well plate. Tagmentation was performed by adding 1 μL of tagmentation mix; 1× tagmentation buffer (10 mM Tris pH 7.5, 5 mM MgCl₂, 5% DMF), 0.003 uL Tagmentation DNA Enzyme 1 (TDE1; Illumina DNA sample preparation kit) to the 1 μL of diluted cDNA per well. Plates were incubated for 10 min at 55° C. before the reaction was stopped by the addition of 0.5 μL 0.2% SDS to each well. Index PCR was carried out after the addition of 3.5 μL custom Nextera Index primers (0.5 μM) by dispensing 2 μL of PCR mix; 1× Phusion Buffer (Thermo Fisher Scientific), 0.01 U pl-1 of Phusion DNA polymerase (Thermo Fisher Scientific), 0.025% Tween-20, 0.2 mM dNTP each. PCR was performed out at 3 minutes at 72° C.; 30 seconds at 95° C.; 12 cycles of (10 seconds at 95° C.; 30 seconds at 55° C.; 1 minute at 72° C.); and 5 minutes at 72° C. Each indexed library plate was pooled by spinning out gently using a 300-ml robotic reservoir (Nalgene) fitted with a custom scaffold by pulsing the centrifuge to <200 g. The pooled libraries were afterwards purified with custom carboxylated magnetic beads in 22% PEG solution at a ratio of 1 sample to 0.7 beads.

### Sequencing of Smart-Seq3Xpress Libraries

Smart-seq3xpress libraries were sequenced on a MGI DNBSEQ G400RS platform. Prior to sequencing on MGI platform ready circular ssDNA libraries were generated using the MGIEasy Universal Library Conversion Kit (MGI). Adapter conversion PCR was carried out on 50 ng of final pooled library for 5 cycles, following circularization of 1 pmol dsDNA according to manufacturer's protocol. DNA nanoballs (DNBs) were created from 80 fmol of circular ssDNA library pools using a custom rolling-circle amplification primer (5′-TCGCCGTATCATTCAAGCAGAAGACG-3′, IDT). DNBs were sequenced 100 bases paired end (PE100) using custom sequencing primers (Read 1: 5′-TCGTCGGCAGCGTCAGATGTGTATAAGAGACAG-3′; MDA: 5′-CGTATGCCGTCTTCTGCTTGAATGATACGGCGAC-3′, Read 2: 5′-GTCTCGTGGGCTCGGAGATGTGTATAAGAGACAG-3′; i7 index: 5′-CCGTATCATTCAAGCAGAAGACGGCATACGAGAT-3′; i5 index: 5′-CTGTCTCTTATACACATCTGACGCTGCCGACGA-3′).

### Data Preprocessing

Raw FASTQ files were processed with zUMIs 2.9.7 pipeline (4). UMI-containing reads were identified by the (ATTGCGCAATG) pattern allowing up to two mismatches and reads were filtered for low quality UMIs (3 bases<phred 20) and index barcodes (4 bases <phred 20), before mapped to the human genome (hg38) using STAR (5) version 2.7.3. Read counts, detected genes, and umicounts were calculated using Ensembl gene annotations (GRCh38.95). Down sampled data was generated via zUMIs preprocessing.

### REFERENCES (EXAMPLE 3)

1. Hagemann-Jensen, M., Ziegenhain, C. & Sandberg, R. Scalable single-cell RNA sequencing from full transcripts with Smart-seq3xpress. Nat Biotechnol 40, 1452-+ (2022).
2. Hagemann-Jensen, M., et al. Single-cell RNA counting at allele and isoform resolution using Smart-seq3. Nat Biotechnol 38, 708-+ (2020).
3. Picelli, S., et al. Full-length RNA-seq from single cells using Smart-seq2. Nat Protoc 9, 171-181 (2014).
4. Parekh, S., Ziegenhain, C., Vieth, B., Enard, W. & Hellmann, I. zUMIs—A fast and flexible pipeline to process RNA sequencing data with UMIs. Gigascience 7(2018).
5. Dobin, A., et al. STAR: ultrafast universal RNA-seq aligner. Bioinformatics 29, 15-21 (2013).

### EXAMPLE 4—STRESS TEST OF CHEMICAL RNASE INHIBITOR SOLUTION

### Results and Discussion

To assess several aspects of commercialization capacity and product integrity we tested both a variety of buffers and stress treatments that polyvinyl sulfonic acid (PVSA) could be subjected to. We simulated conditions that would either be part of a variety of methodologies that PVSA might be used for in the proposed applications, or mimicking extreme conditions that PVSA could be subjected to during shipping. After these chemical or physical treatments, these samples were subsequently used as RNase inhibitor in Smart-seq2 (1) library preparation using 100 pg of total RNA as input. Acidic and basic PVSA solutions were made via pH adjusting with HCl and NaOH. We found PVSA stored in a pH as low as 4 or as high as 10 had no negative effect on cDNA library yield compared to the inhibitor itself (pH ˜6.5) (FIG. 14a-b). Similar results were obtained with PVSA stored in Tris, a common buffer in enzymatic reactions, at 1 or 5 mM, or pH7 or 8 (FIG. 14c). We also subjected aliquots of PVSA to a variety of temperatures from −20° C. to 50° C. for 12 or 24 hours, mimicking reasonable extreme shipping temperatures, before using the PVSA as RNase inhibitor in Smart-seq2 library preparation, importantly with no observed perturbation in cDNA yield (FIG. 14e-f). Rigorously vortexing PVSA for 12 or 24 hours or subjecting PVSA to multiple freeze-thaws of PVSA also did not significantly affect cDNA yield (FIG. 14g-h).

Together, this demonstrates that PVSA is very robust and resilient to physical stress in remaining a potent RNase inhibitor in RNA-sequencing application.

### Methods

Different pH compositions of the chemical RNase inhibitor PVSA were made by the addition of HCl or NaOH to a more concentrated stock of PVSA and diluted with water to the appropriate respective concentrations to make an inhibitor solution with pH of either 4 or 10, respectively, as measured by a pH meter (final concentration is 6.7× stock for lysis buffer). To make Tris buffered inhibitor solutions, a 1M stock of Tris (pH 7 or 8) was added with a more concentrated stock of PVSA and diluted to the appropriate concentrations. For stress tests of temperature and freeze-thaw, aliquots of 250 μl of a PVSA stock were stored at various temperatures (−20° C., 4° C., 37° C., 50° C.) for 12 and 24 hours. For vortexing experiments, vials of PVSA stock were wrapped with parafilm onto a Vortex Genie mixer for 12 and 24 hours. For freeze-thaw experiments, aliquots of PVSA were stored at −20° C. for a minimum of 4 hours, removed to let thaw to 25° C. (room temperature) (1× freeze thaw), and then put back at −20° C. again for the next freeze thaw, and the process was repeated up to 6 times. Different inhibitor solutions and treatments were then used to generate Smart-seq2 cDNA libraries from 100 pg of purified mouse tail tip fibroblast (MTTF) RNA with 30 μg/mL of PVSA in the lysis buffer and 18 cycles in the cDNA amplification step as described in detail in Example 1. Amplified cDNA was bead-purified and run on a Bioanalyzer 2100 High Sensitivity DNA chip.

### REFERENCES (EXAMPLE 4)

1. Picelli, S., et al. Full-length RNA-seq from single cells using Smart-seq2. Nat Protoc 9, 171-181 (2014).

### EXAMPLE 5—ADDITIONAL RNASE-INHIBITING COMPOUNDS IDENTIFIED TO BE

### COMPATIBLE WITH SINGLE-CELL RNA-SEQUENCING LIBRARY PREPARATION

### Results and Discussion

Our initial identification of various sulfonated, sulfated, phosphonated, and carboxylated polymers and monomers polymers and monomers capable of replacing recombinant RNase inhibitor (RI) in single-cell RNA-seq library generation in addition to PVSA; heparin, sodium alginate, vinyl sulfonic acid (VSA), Dextran Sulfate, Fucoidan, and 2-(N-morpholino) ethanesulfonic acid (MES) (Example 1); motivated us to further characterize libraries generated using these compounds as RNase inhibitor in high-throughput sequencing. We used 100 pg of purified mouse fibroblast RNA as template and generated Smart-seq2 cDNA libraries using various amounts of alternative inhibitor in the lysis buffer (FIG. 15). When comparing cDNA yield of the original Smart-seq2 protocol utilizing recombinant RNase inhibitor, or 30 μg/mL PVSA (optimal range established in Example 1) we identified concentration ranges for heparin, sodium alginate, VSA, and dextran sulfate that yielded the maximum amount of cDNA for each alternative inhibitor, and these yields maximum were similar to the yield of the original Smart-seq2 protocol and the yield obtained by the “optimal range” found with PVSA (30-60 μg/mL PVSA) (FIG. 15). The optimal concentrations for the additional alternative inhibitors were 2-10 μg/mL for heparin, 100-400 μg/mL for sodium alginate (NaAlg), 500-2000 μg/mL for VSA, 1.5-2.5 μg/mL for dextran sulfate, 5-20 μg/mL for fucoidan, and 6-8 mg/mL for 2-(N-morpholino)ethanesulfonic acid (MES) (FIG. 15a-i). High-throughput sequencing of cDNA libraries generated from these conditions revealed that the optimal concentrations selected by inspecting cDNA yield on Bioanalyzer traces corresponded well with the highest percent of reads uniquely mapped to the genome (FIG. 15j) and the number of genes detected in that condition (FIG. 15k), and was reflected in genomics mapping statistics to exonic, intronic, and intergenic regions (FIG. 15l). Thus, we identified six additional polymers and monomers capable of replacing recombinant RNase inhibitor in single-cell RNA-seq lysis buffer and library preparation and their optimal concentration ranges for the application.

### Methods

Alternative chemicals: heparin (cat. H6279, Sigma), sodium alginate (cat. A1112, Sigma VSA (cat. 278416, Sigma), dextran sulfate (4031, Sigma), fucoidan (cat. F1819, Sigma), MES (cat. 69892, Sigma); along with PVSA were tested as potential inhibitors following Smart-seq2 protocol with 100 pg of purified MTTF RNA and 18 cycles of amplification. The standard Smart-seq2 protocol (SS2-RI) using recombinant inhibitor (cat. no. 2313B, Recombinant RNase Inhibitor, TaKaRa) and no inhibitor were used as controls. Smart-seq2 libraries using the alternative RNase inhibitors tested were prepared as described for PVSA in Example 1 but replacing PVSA with the respective alternative chemical. Final libraries were sequenced on an Illumina Nextseq550 and mapped to the Gencode GRm38 mouse genome using STAR (1). STAR aligner determined the accuracy of mapping for each sample with percent uniquely mapped reads. The zUMIs (2) pipeline was used to obtain number of genes detected per sample (featureCounts). The fraction of reads that mapped to exonic, intronic, or intergenic regions in the genome were obtained with Qualimap's RNASeQC (3,4).

### REFERENCES (EXAMPLE 5)

1. Dobin, A., et al. STAR: ultrafast universal RNA-seq aligner. Bioinformatics 29, 15-21 (2013).
2. Parekh, S., Ziegenhain, C., Vieth, B., Enard, W. & Hellmann, I. zUMIs—A fast and flexible pipeline to process RNA sequencing data with UMIs. Gigascience 7(2018).
3. Okonechnikov, K., Conesa, A. & Garcia-Alcalde, F. Qualimap 2: advanced multi-sample quality control for high-throughput sequencing data. Bioinformatics 32, 292-294 (2016).
4. Garcia-Alcalde, F., et al. Qualimap: evaluating next-generation sequencing alignment data. Bioinformatics 28, 2678-2679 (2012).

### EXAMPLE 6—REPLACING RECOMBINANT RNASE INHIBITOR WITH POLYVINYL SULFONIC ACID IN THE USE OF COMMERCIALLY AVAILABLE KITS FOR BULK RNA-SEQ LIBRARY PREPARATION

### Results and Discussion

To investigate the usefulness of polyvinyl sulfonic acid PVSA) as RNase inhibitor in additional RNA-seq workflows, we investigated if PVSA would be compatible with commercially available kits for bulk RNA sequencing, replacing recombinant RNase inhibitor in these workflows. We tested replacing conventional RNase inhibitor with PVSA together with three commercially available RNA-seq kits: NEBNext RNA Sequencing Kit (New England Biolabs), TruSeq RNA Sample Preparation Kit (Illumina), and QIAseq FX Single Cell RNA Library Kit (Qiagen). We selected these kits for testing due to their differences in sample preparation steps, and their deviation from the procedures used in Smart-seq2, Smart-seq3, and Smart-seq3xpress, which we had previously characterized in depth (Example 1 and Example 3). We added a range of PVSA concentrations (0, 2.4, 6 μg/mL) or recombinant RNase inhibitor (RI, according to instructions for respective RNA-seq kit) to the lysis step for the NEBNext RNA Sequencing Kit and observed that RNA library yield was unaffected by the addition of PVSA (FIG. 16a). Similarly, we added PVSA (0, 1.2, 3, 12, 30, or 120 μg/mL) to the denaturation step of the QIAseq FX Single Cell RNA Library Kit with no effect on library yield (FIG. 16b). Lastly, we tested different concentrations of PVSA (0, 1.5, 15, or 150 mg/mL) in the RNA sample solution prior to RNA bead purification using the TruSeq RNA Sample Preparation Kit, and even at the highest PVSA concentrations RNA library yield was not perturbed (FIG. 16c). We sequenced the samples on an Illumina Nextseq550 and mapped the resulting reads using STAR aligner. The output from featureCounts analysis showed that for the three kits, addition of PVSA did not affect the number of genes detected per sample (FIG. 16d). Additional alignment statistics from STAR such as percent reads uniquely mapped, percent of genes too short to be mapped, and rate ate of mismatch within a read, insertion length within a read, and deletion length within a read revealed that PVSA does not affect the quality and mappability of the reads (FIG. 16e-i). Additional analyses of reads mapped within the genome via percentage of reads which are exonic, intronic and intergenic (Qualimaps) and the read quality mapped along the transcript with base matching, base insertion, base deletion, and base substitution (BBMap) further showed that PVSA does not perturb the quality of these kit-derived RNA libraries. These results show that PVSA is compatible with a range of commercially available bulk-RNA-seq kits handling samples with high RNase content. Thus, PVSA can substitute an already provided recombinant RNase inhibitor (sold in the NEBNext kit), or be used as an addition inhibitor with the kit (as is the case QIAseq FX and TruSeq) to ensure RNA preservation for bulk and single-cell RNA-sequencing kits.

### Methods

RNA sequencing libraries were generated using three commercial kits: NEBNext RNA Sequencing Kit (#E6420S, New England Biolabs), QIAseq FX Single Cell RNA Library Kit (cat. 180735, Qiagen), and TruSeq RNA Sample Preparation Kit (cat. RS-122-2002, Illumina), following the instructions provided in the respective kits while replacing recombinant RNase inhibitor with PVSA or adding PVSA into the earliest feasible step when adding the RNA (manual version 5.05/20, 2016/06, and 15026495 Rev. F, for NEBNext, QiaseqFX, and TruSeq respectively). For the NEBNext RNA Sequencing Kit, 100 pg of purified mouse tail tip fibroblast (MTTF) total RNA was used with the addition of different amounts of PVSA (0, 2.4, 6 μg/mL) or the protocol recommended amount of recombinant RNase inhibitor NEB (RNase Inhibitor, Murine, E6429) added to the lysis step (Section 1, Step 1.2 in the manual). For the QIAseq FX Single Cell RNA Library Kit, 100 pg of purified MTTF RNA was used with the addition of different amounts of PVSA (0, 1.2, 3, 12, 30, 120 μg/mL) added prior to the denaturation step (Amplification of Purified RNA, Step 1). For RNA sequencing libraries generated from the TruSeq RNA Sample Preparation Kit, 100 ng of purified MTTF RNA was used with different amounts of PVSA (0, 1.5, 15, 150 mg/mL) added to the RNA prior to RNA purification (Chapter 3, Make RBP, Step 1). Final libraries were sequenced on an Illumina Nextseq550 and mapped to the Gencode GRm38 mouse genome using STAR (1). STAR aligner determined the accuracy of mapping for each sample with percent uniquely mapped reads. The zUMIs (2) pipeline was used to obtain number of genes detected per sample (featureCounts). The fraction of reads that mapped to exonic, intronic, or intergenic regions in the genome were obtained with Qualimap's RNASeQC (3,4). The fraction of bases matching to the genome along the read length were obtained through BBMap.

### REFERENCES (EXAMPLE 6)

1. Dobin, A., et al. STAR: ultrafast universal RNA-seq aligner. Bioinformatics 29, 15-21 (2013).
2. Parekh, S., Ziegenhain, C., Vieth, B., Enard, W. & Hellmann, I. zUMIs—A fast and flexible pipeline to process RNA sequencing data with UMIs. Gigascience 7(2018).
3. Garcia-Alcalde, F., et al. Qualimap: evaluating next-generation sequencing alignment data. Bioinformatics 28, 2678-2679 (2012).
4. Okonechnikov, K., Conesa, A. & Garcia-Alcalde, F. Qualimap 2: advanced multi-sample quality control for high-throughput sequencing data. Bioinformatics 32, 292-294 (2016).

### EXAMPLE 7—INCREASED YIELD IN CDNA LIBRARY GENERATION UPON INCLUSION OF POLYVINYL SULFONIC ACID RESULTS MAINLY FROM TO RNASE INHIBITION

### Results and Discussion

Our previous experiments had shown that supplying polyvinyl sulfonic acid (PVSA) to the RNA samples, within defined PVSA concentration ranges, inhibits RNA degradation of stored single-cell RNA and bulk RNA and that it is directly compatible with following single-cell and bulk RNA-sequencing library preparation, and furthermore that this does not increase base errors in the sequencing libraries (Example 1). Although our experiments suggested that increased cDNA yield upon PVSA including in single-cell RNA-seq library preparation is primarily due to inhibition of RNA degradation, we here performed additional experiments to explore its mode of action. To investigate whether increased cDNA yield could come from improving either the reverse transcription or DNA amplification steps, we designed an experiment where we spiked varying amounts of PVSA into the reaction at different steps of RNA-seq library preparation procedure. To this end, we set up an experiment of conventional Smart-seq2 including recombinant RNase inhibitor in both the lysis buffer and in the following reverse transcription (RT) step, but in addition to this we also added PVSA either in the lysis buffer before adding reverse transcriptase (i.e. before RT), or, after completion of RT but before the following cDNA amplification by PCR. Because recombinant RNase inhibitor in the established Smart-seq2 protocol is present in sufficient amount to inhibit small amounts RNases present in purified RNA, we argued that any additional increase in cDNA yield by PVSA would indicate enhancing either the reverse transcriptase reaction, or, DNA polymerase reaction. In the first setup, 100 ng of purified RNA from cultured human embryonic kidney cells (HEK293FT) was used as starting material and 0, 30, 60, 120, 180, 240, 360, or 480 ng/μL PVSA was included in the Smart-seq2 lysis buffer in addition to 0.9 Units/μL recombinant RNase inhibitor (TaKaRa). First-strand cDNA synthesis was performed using Superscript II reverse transcriptase (Thermo Fisher) in accordance with the Smart-seq2 protocol, and upon completion, we purified the first-strand cDNA from the RT buffer using AMPure XP magnetic beads (Beckman Coulter) with elution in 10 mM Tris buffer. We subsequently performed Smart-seq2 cDNA amplification and analyzed the resulting double-stranded cDNA library on Bioanalyzer 2100 dsDNA HS chips (Agilent). We observed that cDNA yield was not increased upon supplementing PVSA on top of recombinant RNase inhibitor in RT step, but instead the yield decreased as more PVSA was added (FIG. 17a), reflecting inhibition of RT at high concentrations as indicated earlier in Example 1. Importantly, these results suggest that the high yield of cDNA in the optimal range of 30-60 ng/μl PVSA in Smart-seq2 lysis buffer which we had identified earlier results from RNA preservation rather than enhancing the activity the reverse transcriptase e.g., via molecular crowding in the RT this step. To test the effect of including PVSA in the PCR step, we performed normal Smart-seq2 first-strand cDNA synthesis using recombinant RNase inhibitor (TaKaRa) and upon RT completion, but before PCR amplification, we added various amounts of PVSA resulting in the final concentrations of 5, 11, 22, 32, 43, 65, 86 ng/μL PVSA in the PCR (Methods). Addition of PVSA in PCR resulted in decreased yield in reactions towards the highest concentrations of PVSA (FIG. 17a), demonstrating that PVSA does not only inhibit RT at high concentrations but also inhibits PCR. Additionally, we tested if high amounts also of recombinant RNase inhibitor might have an inhibitory effect on yield at RT and PCR step. When we add 0.1 pl of RRI to the lysis step, we observed a decrease in cDNA yield (FIG. 17c), indicating an inhibitory effect when recombinant RNase inhibitor is added in excess to the reaction.

To investigate whether PVSA interacts with RNase proteins, thereby possibly inhibiting these enzymes, and/or whether it interacts with RNA molecules, thereby possibly decreasing degradation by protecting or binding the RNA, we performed Nuclear Magnetic Resonance spectroscopy (NMR) on bovine RNase A or a previously well-characterized RNA (14-mer cUUCGg tetraloop hairpin RNA)² in absence and presence of PVSA. We performed ¹H-¹⁵N heteronuclear multiple-quantum coherence (HMQC) spectroscopy on Bovine RNase A (1370 μg/μL) without and with the PVSA ligand (12 μg/μl) in 0.8×DPBS buffer pH 7.4 at 25° C., following 1 hour incubation (Methods). The ¹H-¹⁵N HMQC spectra showed drastic changes of RNase A upon addition of the PVSA ligand (FIG. 18). Apart from a few residues including for example G68, K91 and L51, all in flexible loops, most amide peaks, representing the protein backbone, broadened beyond detection upon the addition of PVSA ligand, indicating an extensive interaction between RNase A and PVSA molecules. This large broadening could be the result of increased molecular weight, when the ligand binds to RNase A, or due to a change in the protein structure, connected with chemical shift changes on the intermediate exchange timescale, both indicating RNase:PVSA interaction. As sample aggregation was not observed, and flexible residues remain observable, RNase A is likely entering a lower polymeric, soluble state.

Next, we compared the 1H-1D spectra of a 14-mer cUUCGg tetraloop hairpin RNA (504 μg/μL) (FIG. 19a) in the absence and presence of PVSA ligand (12 μg/μl). Hydrogen atoms in the nucleobase, H6, H2, and H8s (FIG. 19b) and imino protons, indicative for base-pairing (FIG. 19c) showed no notable change when the ligand was added. This indicates that the ligand does not intercalate or change the structure of the RNA. Finally, the comparison of ³¹P-1D spectra (FIG. 19d) showed that the phosphate backbone signals also had no mentionable chemical shift changes, besides normal variation between same samples, upon addition of the PVSA ligand, altogether indicating the lack of notable interactions between PVSA and the RNA.

Although these experiments do not fully characterize interactions between RNase A and PVSA or RNA and PVSA, the present data indicate strong effects on RNase A protein and little or no effect on RNA. Together, this is in line with our functional data, indicating that PVSA inhibits RNA degradation primarily by RNase inhibition.

Finally, the lack of interaction between PVSA and RNA also indicates that PVSA could be useful as an agent for protecting RNA from RNases in NMR applications characterizing RNA structure, folding, and dynamics. This is important because recombinant RNase inhibitor proteins may interact with RNA affecting its spectra in NMR.

### Methods

Reverse Transcription and cDNA Amplification Experiments

Amplified cDNA was performed according to the standard Smart-seq2 protocol (1) with some modifications. For PVSA added to the reverse transcription step, purified RNA (100 ng of HEK293FT RNA was added to the Smart-seq2 lysis buffer (0.1% Triton X-100, 2.5 mM dNTP/each, 2.5 mM Smart-seq2 oligo-dT (5′-AAGCAGTGGTATCAACGCAGAGTACT30VN-3′), and 4 U Recombinant RNase Inhibitor (cat. no. 2313B, TaKaRa), that contained a range of PVSA added to the buffer (0, 30, 60, 120, 240, 360, or 480 ng/μL). RNA in the lysis buffer was incubated at 72° C. for 3 min. The reverse transcription reaction buffer consisted of 1× Superscript II buffer, 5 mM DTT, 1M betaine (cat. no. B0300, Sigma), 10 mM MgCl2, 1 uM Smart-seq2 TSO, 10 U Superscript II (cat. no. 18064-014, Invitrogen), and 10 U Recombinant RNase inhibitor (cat. no. 2313B, TaKaRa). RT thermocycles were 42° C. for 90 min, followed by 10 cycles of 42° C. for 2 min and 50° C. for 2 min. Then the first strand cDNA was purified using AMpureXP beads (cat. no. A63881, Beckman Coulter), washed twice with 70% ethanol, and eluted in 10 mM Tris in 10 pl. The amplification reaction mixture contained the 10 μl of purified cDNA and 1× KAPA HiFi HotStart Ready Mix (cat. no. KK2602, Roche Diagnostics) and 80 nM ISPCR primers (5′-AAGCAGTGGTATCAACGCAGAGT-3′) in a total reaction volume 25 μL and thermocycles for Smart-seq2 cDNA amplification were 98° C. for 3 min, followed by 12 cycles of 98° C. for 20 sec, 67° C. for 15 sec, and 72° C. for 6 min, followed by a final incubation at 72° C. for 5 min. For PVSA added to the amplification step, 5 ng of HEK293FT RNA was added to the Smart-seq2 lysis buffer, and lysis and reverse transcription were carried out as normal. No bead purification was carried out between the reverse transcription and amplification steps. At the amplification step, a range of PVSA concentrations (final concentrations 5, 11, 22, 32, 43, 65, or 86 ng/μL) were added to the samples along with the HotStart Ready Mix and ISPCR primers. Samples were amplified with 10 cycles (cycles described above). For experiments where PVSA was added at the amplification step to test DNA polymerase inhibition, the standard Smart-seq2 protocol and 5 ng of purified HEK293FT RNA was used (containing 4 U of Recombinant RNase inhibitor in the lysis mix and an additional 10 U of Recombinant RNase Inhibitor in the reverse transcription mix), and cDNA was amplified with 10 cycles. Additional Recombinant RNase inhibitor spiking during Smart-seq2 cDNA library generation occurred either during the lysis step (an additional 0.1 μl/4 U of recombinant RNase inhibitor) or reverse transcriptase step (an additional 0.05 μl/2 U μl of recombinant RNase inhibitor).

### NMR Experiments

Bovine RNase A (Machery-Nagel) in lyophilized form was resuspended in DPBS (Thermofisher), then supplemented with 10% D₂O (Merck). 14-mer cUUCGg tetraloop hairpin RNA (sequence 5′-GGCACUUCGGUGCC-3′) (2) was prepared by solid-phase-synthesis followed by DMT column purification (Glen Pak). The sample was resuspended in H₂O and then diluted in 15 mM NaP, 25 mM NaCl, 0.1 mM EDTA pH 6.5 and refolded by heating to 95° C. for 2 minutes, then snap-cooled on ice for 30 minutes, and supplemented with 10% D₂O. Samples were transferred into Shigemi tubes and ligand was added from a stock concentration of 380 μg/μl to a final concentration of 12 μg/μl, incubated at room temperature for 1 hour prior. NMR measurement: Spectra acquired on 600 MHz Bruker with 5 mm TXI-HCNP Cryo-probe. SOFAST HMQC (3) acquired with ¹H(N) centered at 9 ppm with 6 ppm bandwidth, and ¹⁵N centered at 120 ppm with 50 ppm bandwidth with 1024 scans. 1H-1D was acquired with excitation sculpting with 16 scans and 1D-³¹P applying phosphate buffer suppression with 1024 scans. All spectra were acquired at 298K. Analysis performed in Topspin 3.6. Assignment was performed based on published RNase A spectra from Tonelli M et. al. (4).

### REFERENCES (EXAMPLE 7)

1. Picelli, S., et al. Full-length RNA-seq from single cells using Smart-seq2. Nat Protoc 9, 171-181 (2014).
2. Nozinovic, S., Furtig, B., Jonker, H. R. A., Richter, C. & Schwalbe, H. High-resolution NMR structure of an RNA model system: the 14-mer cUUCGg tetraloop hairpin RNA. Nucleic Acids Res 38, 683-694 (2010).
3. Schanda, P., Kupce, E. & Brutscher, B. SOFAST-HMQC experiments for recording two-dimensional heteronuclear correlation spectra of proteins within a few seconds. J Biomol Nmr 33, 199-211 (2005).
4. Tonelli, M., et al. Assignments of RNase A by ADAPT-NMR and enhancer. Biomol Nmr Assign 9, 81-88 (2015).

### EXAMPLE 8—STORING AND PRESERVING RNA FROM BIOLOGICAL MATERIAL ON PVSA-COATED PAPER

### Results and Discussion

After our preliminary results of preserving RNA from biological material on PVSA-coated paper sheets (Example 2), we here conducted a larger-scale experiment to test the efficacy of this sample preservation over time (FIG. 20). To prepare the substrate paper, we soaked western blotting cotton fiber paper (iBlot Filter Paper) in various concentrations of PVSA (0 μg/ml, 3 μg/mL, 30 μg/mL, 300 μg/mL, 3 mg/ml, or 30 mg/ml). The sheets were subsequently air dried and cut into 40×5 mm strips. We cultured and lysed 1 million human embryonic kidney cells (HEK293FT) using 1% Triton-X, a common detergent capable of lysing cell membranes (1). We spotted 2.5 pl of this cell lysate (equivalent to 2500 lysed cells) onto the substrate strips and incubated these at room temperature (25° C.) for 1, 3, or 7 days, upon which they were subsequently eluted in water (Methods). The eluate was either immediately processed to make cDNA libraries using a bulk version of Smart-seq2 (FIG. 20a-c), or the eluate was left at room temperature for an additional 6 days before cDNA generation (FIG. 20d) (Methods). The conditions with the highest concentrations of PVSA (3 mg/ml, or 30 mg/ml) yielded the most cDNA for all samples tested, producing intact the characteristic cDNA peak at around 2 kb present in intact full-length cDNA libraries, while sheets soaked with lower concentrations of PVSA yielded more degraded profile; indicating a PVSA-concentration-dependent shielding of the human embryonic kidney cell RNA. These results show that RNA preservation using paper-based spotting methods could be a viable approach for storing biological samples for subsequent RNA-sequencing when the paper is pre-treated with PVSA.

### Methods

Strips of western blotting cotton fiber paper (iBlot Filter Paper) were pre-incubated in a solution of 0 μg/ml, 3 μg/mL, 30 μg/mL, 300 μg/mL, 3 mg/ml, or 30 mg/ml PVSA for 15 minutes, air-dried before use. A frozen pellet of one million HEK293FT cells were diluted and lysed in 1000 μl of 1% Triton-X100. The sheets were spotted with 2.5 pl of lysed cell solution (2500 cells). Paper strips were incubated for 1, 3, or 7 days at room temperature, after which they were eluted in 100 μl of water. 1 μl of eluate (representing approximately 25 cells input if assuming no material bound into the paper) were used for cDNA synthesis following the Smart-seq2 protocol as detailed in Example 1 with minor modifications. Briefly, the 1 μl of eluate was added to Smart-seq2 lysis buffer containing 0.1% Triton X-100, 2.5 mM dNTP/each (cat. no 10297018, Invitrogen), and 2.5 mM Smart-seq2 oligo-dT and omitting Recombinant RNase Inhibitor. Samples were then incubated at 72° C. for 3 min. Reverse transcription mix was added which contained 1× Superscript II buffer, 5 mM DTT, 1M betaine (cat. B0300, Sigma), 10 mM MgCl₂, 1 uM Smart-seq2 TSO (IDT), and 10 U Superscript II (cat. no. 18064-014, Invitrogen), omitting RRI, and reverse transcription was performed according to Smart-seq2 protocol. After KAPA HiFi DNA polymerase and ISPCR amplification primers were added cDNA amplification was performed for 18 cycles. Control cDNA samples were prepared by directly adding 2.5 pl of lysed cells from the 1% Triton-X100 starting solution into 100 μl of water and adding 1 μl of that to the SS2 protocol with 18 cycles of cDNA amplification. It should be noted that a higher yield is expected in this control since no material would be trapped inside the paper sheet. An additional cDNA library was prepared from the day 1 eluted sample where the eluate was incubated at room temperature for 6 more days (1+6).

### REFERENCES (EXAMPLE 8)

1. Brown, R. B. & Audet, J. Current techniques for single-cell lysis. J R Soc Interface 5, S131-S138 (2008).

### Items

1. A method of preparing a cDNA sequencing library from RNA, characterised in that the method includes the use of an agent selected from the group consisting of: a sulfonated and/or carboxylated polymer; a sulfonated and/or carboxylated monomer; and a functionalised polysaccharide.
2. The method according to Item 1, comprising:
   i. liberating a plurality of RNA molecules from one or more cells or cell extract in the presence of an aqueous solution comprising the agent;
   ii. synthesizing a plurality of cDNA strands from the RNA molecules by reverse transcription; and
   iii. processing the cDNA strands to generate a cDNA library for sequencing.
3. The method according to Item 2, wherein step (ii) comprises hybridizing a first strand cDNA synthesis primer to an RNA molecule and synthesizing a first cDNA strand complementary to at least a portion of the RNA molecule by reverse transcription.
4. The method according to Item 3, wherein synthesizing a first cDNA strand comprises the use of a first strand synthesis primer selected from: an oligo-dT primer, a primer with a random sequence, a degenerate primer specific to a gene family, a gene specific primer, and/or a primer complementary to a pre-ligated oligo.
5. The method according to Item 3 or 4, wherein the first strand cDNA synthesis primer comprises a tag, such that synthesizing the first cDNA strand incorporates the tag into the cDNA to provide a tagged cDNA strand, and wherein the tag comprises a unique molecular identifier (UMI) sequence and/or a barcode.
6. The method according to any one of Items 3-5, wherein when a plurality of RNA molecules are released from one or more cells, the one or more cells are spatially separated into single cells prior to the release of RNA molecules such that a plurality of individual RNA samples is provided, and wherein each individual RNA sample comprises a plurality of RNA molecules from a single cell.
7. The method according to Item 6, wherein step (ii) comprises hybridizing a first strand cDNA synthesis primer to an RNA molecule and synthesizing a first cDNA strand complementary to at least a portion of the RNA molecule by reverse transcription,
   wherein the cDNA synthesis primer comprises a tag, and synthesizing the first cDNA strand thereby incorporates the tag into the cDNA to provide a plurality of tagged cDNA samples, wherein the cDNA in each tagged cDNA sample is complementary to RNA from a single cell, and wherein the tag comprises a unique molecular identifier (UMI) sequence and/or a barcode.
8. The method according to any one of Items 2-7, wherein the agent is present in the first cDNA strand synthesis reaction at a concentration of 1-4000 μg/mL.
9. The method according to any one of Items 2-8, wherein the method further comprises second cDNA strand synthesis from the first cDNA strand.
10. The method according to Item 9, wherein second strand synthesis comprises RNA nicking and displacement; a primer that is complementary to an adapter pre-ligated to the 5′-end of the RNA template; and/or a template switching oligonucleotide (TSO) primer.
11. The method according to any one of Items 3-10, wherein the cDNA is amplified, optionally via in vitro transcription, or by PCR using a first forward amplification primer and a first reverse amplification primer.
12. The method according Items 2 to 11, wherein step (iii) comprises introducing one or more sequencing platform adapter sequences to the cDNA.
13. The method according to any one of Items 2 to 11, wherein step (iii) comprises fragmentation of the cDNA.
14. The method according to Item 12, wherein step (iii) comprises introducing one or more sequencing platform adapter sequences to cDNA fragments produced by fragmentation of the cDNA.
15. The method according to any one of the preceding items, wherein step (iii) comprises an amplification step optionally via in vitro transcription (IVT) or by PCR using a second forward amplification primer and a second reverse amplification primer.
16. The method according to any one of Items 2-15, wherein the first strand cDNA synthesis primer; and/or the first forward amplification primer; and/or first reverse amplification primer; and/or the TSO; and/or the second forward amplification primer; and/or second reverse amplification primer comprise: a unique molecular identifier (UMI); and/or multiple predefined nucleotides; and/or an amplification primer binding domain; and/or a barcode; and/or an adapter sequence.
17. The method according to any one of the preceding items, wherein the RNA sample comprises poly(A) containing RNA molecules, such as messenger RNA (mRNA) molecules, and the method comprises producing the cDNA sequencing library from mRNA.
18. The method according to any one of the preceding items, wherein the method is for preparing a single cell RNA sequencing (scRNAseq) library.
19. A cDNA library obtained or obtainable by the method of any one of Items 1 to 18.
20. Use of a cDNA library according to Item 17 for RNA sequencing (RNA-seq), such as single cell RNA-seq.
21. Use of an agent selected from the group consisting of: a sulfonated and/or carboxylated polymer; a sulfonated and/or carboxylated monomer; and a functionalised polysaccharide in a method of producing a cDNA sequencing library, or in a method of in situ RNA-sequencing.
22. A method for performing RNA sequencing (RNA-seq), such as single cell RNA-seq (scRNA-seq), the method comprising the steps of: preparing a cDNA sequencing library according to the method of any one of Items 1 to 18; and sequencing the cDNA library.
23. A lysis buffer comprising:
   an agent selected from the group consisting of: a sulfonated and/or carboxylated polymer; a sulfonated and/or carboxylated monomer; and a functionalised polysaccharide;
   a detergent and/or chaotropic agent;
   optionally comprising:
   PEG
   BSA
   RNA spike-in
   dNTPs; and/or
   first strand cDNA synthesis primer.
24. The lysis buffer according to Item 23, wherein at a 1× concentration, the agent is present at 0.1-8000 μg/mL.
25. The method according to any one of Items 1 to 18 and 22, the use according to Item 20 and 21, and the lysis buffer according to Item 23 or 24, wherein the method, use, and lysis buffer does not comprise a biological RNase inhibitor.
26. A solid support comprising the agent as defined in any one of Items 23 to 25.
27. A microreactor droplet comprising lysis buffer as defined in any one of Items 23 to 25.
28. A method for lysing one or more cells and releasing RNA molecules, wherein the method comprises contacting one or more cells with a lysis buffer as defined in any one of Items 23 to 25 in order to provide a plurality of RNA molecules.
29. The method according to Item 28, wherein the one or more cells are spatially separated into single cells prior to contact with the lysis buffer such that a plurality of individual RNA samples is provided, wherein each individual RNA sample comprises a plurality of RNA molecules from a single cell.
30. A kit for preparing a cDNA sequencing library comprising:
   an agent selected from the group consisting of: a sulfonated and/or carboxylated polymer; a sulfonated and/or carboxylated monomer; and a functionalised polysaccharide; and
   a first strand cDNA synthesis primer;
      and optionally
   dNTPs; and/or
   a template switching oligonucleotide (TSO).
31. The method according to any one of Items 1 to 18, 22, 25 or 28, or the use according to Item 20, 21 or 25, or the lysis buffer according to any one of Items 23-25, or the kit according to Item 30, wherein the agent is selected from the group consisting of: polyvinyl sulfonic acid (PVSA), heparin, vinyl sulfonic acid (VSA), sodium alginate, dextran sulfate, fucoidan, 2-(N-morpholino)ethanesulfonic acid (MES), sulfated cellulose, sulfated amylose, sulfated pectic acid, dextran sulfate, sulfated polyvinyl alcohol.

### GENERAL

It should be understood that any feature and/or aspect discussed above in connections with the compounds according to the invention apply by analogy to the methods described herein.

1. An aqueous solution comprising at least 0.1 μg/mL poly(vinylsulfonic acid) (PVSA).

2-65. (canceled)

66. The aqueous solution of claim 1, wherein the concentration of PVSA is between about 0.1 to 2000 μg/mL.

67. The aqueous solution of claim 1, wherein the concentration of PVSA is between about 0.1 to 30 μg/mL, 0.1 to 20 μg/mL, 0.3 to 10 μg/mL, 0.3 to 15 μg/mL, 0.3 to 5 μg/mL, 0.6 to 15 μg/mL, 0.6 to 6 μg/mL, 1.5 to 6 μg/mL, 0.5 to 10 μg/mL, 0.6 to 9 μg/mL, 1 to 8 μg/mL, 3 to 8 μg/mL, 4 to 7 μg/mL, 15 to 120 μg/mL, 30 to 100 μg/mL, 40 to 80 μg/mL, or 50 to 70 μg/mL.

68. The aqueous solution of claim 1, wherein the concentration of PVSA is about 60 μg/mL.

69. The aqueous solution of claim 1, wherein the concentration of PVSA is about 6 μg/mL.

70. The aqueous solution of claim 1, wherein the concentration of PVSA is 0.1 μg/mL.

71. The aqueous solution of claim 1, further comprising an ionic, zwitter-ionic or non-ionic detergent.

72. The aqueous solution of claim 1, further comprising dNTPs and Oligo dT primers.

73. The aqueous solution of claim 1, wherein the detergent is selected from the group consisting of Triton x100, SDS, NP-40/Igepal, Sarkosyl, Tween-20, sodium deoxycholate, and CHAPS.

74. The aqueous solution of claim 1, wherein the aqueous solution consists of PVSA dissolved in water.

75. A method of preparing a cDNA library for single cell RNA-sequencing, the method comprising releasing a plurality of RNA molecules from one or more cells or a cell extract in the presence of the aqueous solution of claim 1.

76. The method of claim 75, wherein the single cell RNA sequencing is droplet-based single-cell RNA-sequencing.

77. The method of claim 75, wherein the single cell RNA sequencing is in situ RNA sequencing.

78. The method of claim 75, wherein the single cell RNA sequencing comprises:
a) isolating a single cell into a lysis buffer comprising a detergent, deoxynucleotide triphosphates (dNTPs), an oligo-dT primer, and poly(vinylsulfonic acid) (PVSA), which lyses the cell and anneals the oligo-dT primer;
b) performing reverse transcription to generate a first-strand cDNA; and
c) amplifying the cDNA;
d) optionally, tagmenting the cDNA using Tn5 enzyme; or
e) optionally, indexing or barcoding the cDNA to create sequencing libraries.

79. The method of claim 76, wherein the concentration of the primers in the amplification is 80 nM.

80. The method of claim 76, wherein the reverse transcription comprises 5 mM dithiothreitol (DTT), 1 mM betaine, 10 mM MgCl₂, 1 mM template-switching oligo (TSO), and 10 U of reverse transcriptase.

81. The method of claim 75, wherein the RNA-sequencing comprises:
a) isolating a single cell into a lysis buffer comprising a detergent, PEG 8000, deoxynucleotide triphosphates (dNTPs), an oligo-dT primer, and poly(vinylsulfonic acid) (PVSA), which lyses the cell and anneals the oligo-dT primer;
b) performing reverse transcription to generate a first-strand cDNA; and
c) amplifying the cDNA;
d) optionally, tagmenting the cDNA using Tn5 enzyme; or
e) optionally, indexing or barcoding the cDNA to create a sequencing library.

82. The method of claim 78, wherein the detergent comprises 0.1% (v/v) Triton X-100, the concentration of dNTPs is 2.5 mM; and the concentration of oligo-dT primer is 2.5 mM.

83. A kit for in situ RNA sequencing, the kit comprising:
a first container comprising an aqueous solution, which comprises poly(vinylsulfonic acid) (PVSA) at a concentration of between about 0.1-120 μg/mL, and
a second container comprising a lysis buffer, deoxynucleotide triphosphates (dNTPs), and primers for reverse transcription, wherein neither the first nor the second container contain a recombinant RNase inhibitor.

84. A multi-well plate comprising a detergent, deoxynucleotide triphosphates (dNTPs), poly(vinylsulfonic acid) (PVSA), and primers for reverse transcription, wherein the concentration of PVSA is between about 0.1-120 μg/mL.
